(19) United States
(12) Patent Application Publication
(10) Pub. No.: US 2024/0293424 A1
(43) Pub. Date: Sep. 5, 2024

(54) CARNITINE-PALMITOYL-TRANSFERASE-1 (CPT-1) INHIBITORS FOR USE IN A METHOD OF PREVENTING OR TREATING SEPSIS IN A MAMMALIAN SUBJECT

(71) Applicant: 2N PHARMA APS, Aalborg (DK)

(72) Inventors: Christoph THIEMERMANN, Aalborg (DK); Preben BRUUN-NYZELL, Aalborg (DK); Shireen MOHAMMAD, Aalborg (DK); Anne SKØTTRUP MØTKHOLT, Aalborg (DK)

(21) Appl. No.: 18/570,866

(22) PCT Filed: Jul. 11, 2022
(86) PCT No.: PCT/EP2022/069258

§ 371 Date: Dec. 15, 2023

(30) Foreign Application Priority Data
Jul. 12, 2021 (EP) 21185051.6

(51) Int. Cl. A61K 31/551; A61P 37/02

(52) U.S. Cl. CPC A61K 31/551; A61P 37/02

(57) ABSTRACT

The present invention relates to Carnitine-Palmitoyl-Transferase-1 (CPT-1) inhibitors for use in a method of preventing or treating sepsis in a mammalian subject. The invention also relates to pharmaceutical compositions comprising a Carnitine-Palmi-toyl-Transferase-1 (CPT-1) inhibitor for said use.

### FIELD OF THE INVENTION

The present invention relates to Carnitine-Palmitoyl-Transferase-1 (CPT-1) inhibitors for use in a method of preventing or treating sepsis in a mammalian subject. The invention also relates to pharmaceutical compositions comprising a Carnitine-Palmitoyl-Transferase-1 (CPT-1) inhibitor for said use.

### BACKGROUND OF THE INVENTION

Sepsis is a potentially life-threatening condition that occurs when the body's response to an infection damages its own tissues. When the infection-fighting processes turn on the body, they cause organs to function poorly and abnormally. Early treatment with antibiotics and intravenous fluids improves chances for survival.

Sepsis may progress to septic shock. Septic shock is a condition in which infection is widely disseminated in many areas of the body, the infection generally being disseminated through the blood from one tissue to another and causing extensive damage. It is a dramatic drop in blood pressure that can lead to severe organ problems and death. Septic shock can occur with numerous medical conditions, including (1) peritonitis caused by the spread of infection from the uterus and fallopian tubes; (2) peritonitis resulting from rupture of the gut, sometimes caused by intestinal disease or wounds; (3) generalized infection resulting from spread of a simple infection; (4) generalized gangrenous infection resulting specifically from gas gangrene bacilli; and (5) infection spreading into the blood from the kidney, urinary tract or the abdomen.

Sepsis is a widespread problem that affects hundreds of thousands of people each year. Sepsis frequently occurs as a hospital-acquired infection, contributing to the significant patient mortality and morbidity, and add significantly to the overall cost of healthcare. A strong and rapid immune response to pathogens is important for preventing, treating and/or reducing the severity of sepsis due to viral, bacterial, and fungal infections.

However, many other cases are acquired outside of hospitals, for example, at home, school, or work. Any infection can lead to sepsis, from, for example, a simple cut, to food poisoning, to a urinary tract infection.

Currently, methods of treating bacterial infections and sepsis primarily consist of applying broad spectrum and/or target-specific antibiotics; however, with the rapid evolution of bacterial strains, many of these drugs become ineffective over time as the bacteria develop resistance to them. Furthermore, once a patient has developed sepsis, the presence of bacterial endotoxins and pyrogenic substances in the body pose the risk of a patient developing more serious conditions, such as septic shock.

EP 3 741 386 A1 describes a method for treating sepsis in a subject, comprising administering a regimen of alpha thymosin peptide to a subject.

WO 2018/011405 A1 describes a non-agonist polypeptide ligand specifically reactive to IL-13Rα1 for use in the treatment or prevention of neutropenia, allergic inflammation, bacteremia and sepsis.

However, compounds that are suitable for the effective prevention or treatment of sepsis have not yet been found. Thus, there is a need for alternative and/or complimentary compounds and methods of preventing or treating sepsis and symptoms thereof. In particular, there is a need for compositions and methods for combatting bacterial infections in, for example, wound patients.

### SUMMARY OF THE INVENTION

The present invention, in one aspect, relates to a Carnitine-Palmitoyl-Transferase-1 (CPT-1) inhibitor for use in a method of preventing or treating sepsis in a mammalian subject.

In one embodiment, the invention relates to a Carnitine-Palmitoyl-Transferase-1 (CPT-1) inhibitor for use as defined herein, wherein the method comprises administering a dosage of Carnitine-Palmitoyl-Transferase-1 (CPT-1) inhibitor to a subject.

In another embodiment, the invention relates to a Carnitine-Palmitoyl-Transferase-1 (CPT-1) inhibitor for use as defined herein, wherein the subject is at risk for the sepsis, severe sepsis or septic shock.

In another embodiment, the invention relates to a Carnitine-Palmitoyl-Transferase-1 (CPT-1) inhibitor for use as defined herein, wherein the Carnitine-Palmitoyl-Transferase-1 (CPT-1) inhibitor is administered prior to, along with and/or after an event predicted to result in pathogen exposure or introduction of an opportunistic environment.

In a preferred embodiment, the Carnitine-Palmitoyl-Transferase-1 (CPT-1) inhibitor is first administered prior to the event, and again on the day of the event, and optionally after the event.

In a particular preferred embodiment, the event is selected from admittance to a hospital or health care facility, surgery, placement of an invasive medical device, kidney dialysis, and initiation of chemotherapy or radiation therapy for cancer treatment.

In another embodiment, the subject is a human.

In another embodiment, the invention relates to a Carnitine-Palmitoyl-Transferase-1 (CPT-1) inhibitor for use as defined herein, wherein the Carnitine-Palmitoyl-Transferase-1 (CPT-1) inhibitor is administered by the oral, sublingual, transdermal or parenteral route, preferably by the intramuscular, intraperitoneal or intravascular route; more preferably by the intravenous route.

In another embodiment, the invention relates to a Carnitine-Palmitoyl-Transferase-1 (CPT-1) inhibitor for use as defined herein, wherein in the method of the invention the Carnitine-Palmitoyl-Transferase-1 (CPT-1) inhibitor is administered within at least the first 24 hours, 48 hours, 72 hours, or 96 hours of showing one or more signs or symptoms of the sepsis, severe sepsis or septic shock.

In another embodiment, the invention relates to a Carnitine-Palmitoyl-Transferase-1 (CPT-1) inhibitor for use as defined herein, wherein the Carnitine-Palmitoyl-Transferase-1 (CPT-1) inhibitor is administered as a single agent.

In another embodiment, the invention relates to a Carnitine-Palmitoyl-Transferase-1 (CPT-1) inhibitor for use as defined herein, wherein the Carnitine-Palmitoyl-Transferase-1 (CPT-1) inhibitor is administered in combination with another drug, preferably in combination with an antimicrobial or an antiviral drug.

In one embodiment, the invention relates to a Carnitine-Palmitoyl-Transferase-1 (CPT-1) inhibitor for use as defined herein, wherein the Carnitine-Palmitoyl-Transferase-1 (CPT-1) inhibitor is a compound of the formula (I) as defined in European patent application no. 21172027.1.

In one embodiment, the Carnitine-Palmitoyl-Transferase-1 (CPT-1) inhibitor for use as defined herein is a compound of the formula (I)

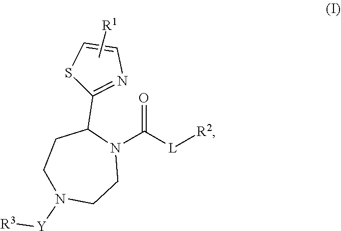

wherein
R¹ is unsubstituted or substituted aryl, preferably unsubstituted or substituted phenyl, naphthyl, tetrahydronaphthyl, indenyl, indanyl, pentalenyl, or fluorenyl,
   unsubstituted or substituted heteroaryl, preferably unsubstituted or substituted pyridyl, pyrazinyl, pyrimidinyl, pyridazinyl, 2-oxo-1,2-dihydropyridinyl, oxazolyl, oxydiazolyl, isoxazolyl, thiadiazolyl, tetrazolyl, pyrazolyl, imidazolyl, thiazolyl and thienyl, quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, or indazolyl,
   unsubstituted or substituted 5- or 6-membered saturated or partially unsaturated heterocyclyl, or
   unsubstituted or substituted C₃-C₈-cycloalkyl, or cyclohexenyl;
L is a single bond, or a difunctional linker, preferably *—O—, *—OCH₂—, *—CH₂O—, *—CH₂—, *—CH₂—CH₂—, *—CH₂—CH₂—CH₂—, or *—CH₂—C(CH₃)₂—, or a trifunctional linker, preferably, *—CH═, wherein the * indicates the point of attachment to the carbonyl (C═O) group;
R² is unsubstituted or substituted phenyl, naphthyl, or pyridyl, or C₁-C₄-alkyl;
R³ is H, C₁-C₈-alkyl, halogen-C₁-C₄-alkyl, or C₃-C₈-cycloalkyl,
   unsubstituted or substituted 4, 5- or 6-membered saturated or partially unsaturated heterocyclyl, or
   unsubstituted or substituted phenyl;
Y is —(C═O)—, —(SO₂)— or a single bond;
or a stereoisomer, a pharmaceutically or veterinarily acceptable salt, hydrate or solvate thereof.

In another embodiment, the Carnitine-Palmitoyl-Transferase-1 (CPT-1) inhibitor for use as defined herein is a compound of the formula (I)

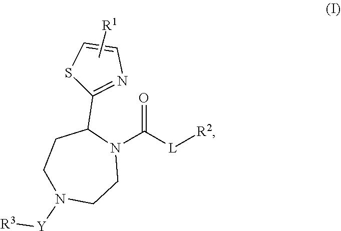

wherein
R¹ is unsubstituted or substituted aryl, preferably unsubstituted or substituted phenyl, naphthyl, tetrahydronaphthyl, indenyl, indanyl, pentalenyl, or fluorenyl,
   unsubstituted or substituted heteroaryl, preferably unsubstituted or substituted pyridyl, pyrazinyl, pyrimidinyl, pyridazinyl, 2-oxo-1,2-dihydropyridinyl, oxazolyl, oxydiazolyl, isoxazolyl, thiadiazolyl, tetrazolyl, pyrazolyl, imidazolyl, thiazolyl and thienyl, quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, or indazolyl,
   unsubstituted or substituted 5- or 6-membered saturated or partially unsaturated heterocyclyl, or
   unsubstituted or substituted C₃-C₈-cycloalkyl, or cyclohexenyl;
L is a single bond, or a difunctional linker, preferably *—O—, *—OCH₂—, *—CH₂O—, *—CH₂—, *—CH₂—CH₂—, *—CH₂—CH₂—CH₂—, or *—CH₂—C(CH₃)₂—, wherein the * indicates the point of attachment to the carbonyl (C═O) group;
R² is unsubstituted or substituted phenyl, naphthyl, or pyridyl,
R³ is H, C₁-C₈-alkyl, halogen-C₁-C₄-alkyl, or C₃-C₈-cycloalkyl,
   unsubstituted or substituted 4, 5- or 6-membered saturated or partially unsaturated heterocyclyl, or
   unsubstituted or substituted phenyl;
Y is —(C═O)—, —(SO₂)— or a single bond;
or a stereoisomer, a pharmaceutically or veterinarily acceptable salt, hydrate or solvate thereof.

In a preferred embodiment, the Carnitine-Palmitoyl-Transferase-1 (CPT-1) inhibitor for use as defined herein is a compound selected from the group consisting of

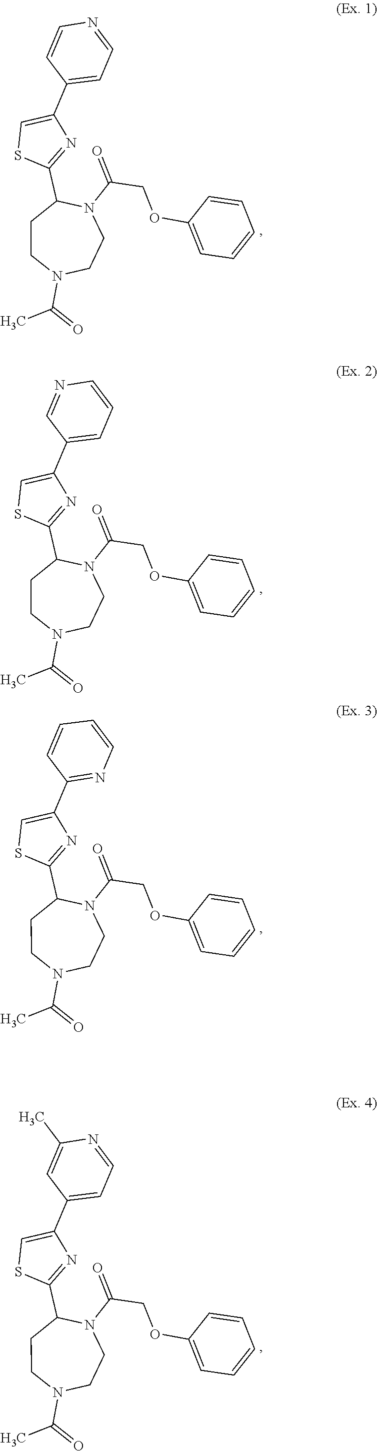

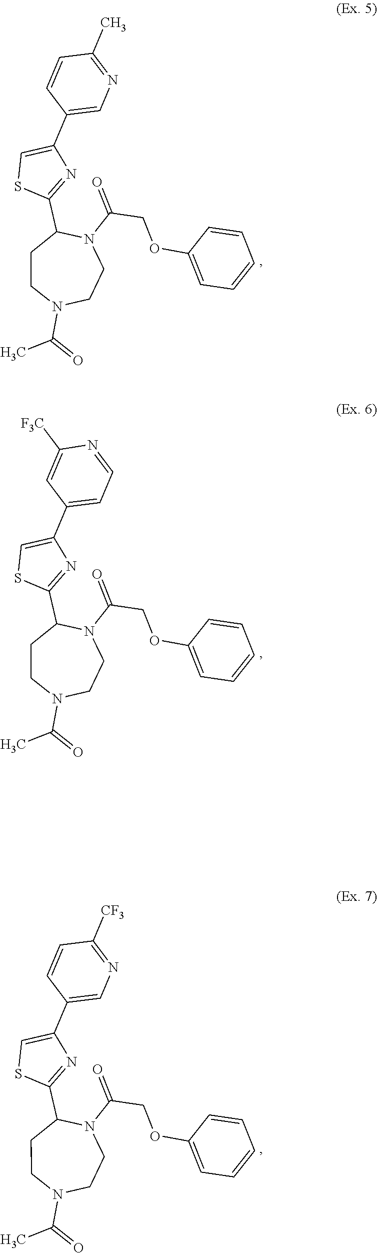 [SMILES: CC(=O)N1CCC(c2nc(-c3ccc(C)nc3)cs2)N(C(=O)COc2ccccc2)CC1 ; CC(=O)N1CCC(c2nc(-c3ccnc(C(F)(F)F)c3)cs2)N(C(=O)COc2ccccc2)CC1 ; CC(=O)N1CCC(c2nc(-c3ccc(C(F)(F)F)nc3)cs2)N(C(=O)COc2ccccc2)CC1]

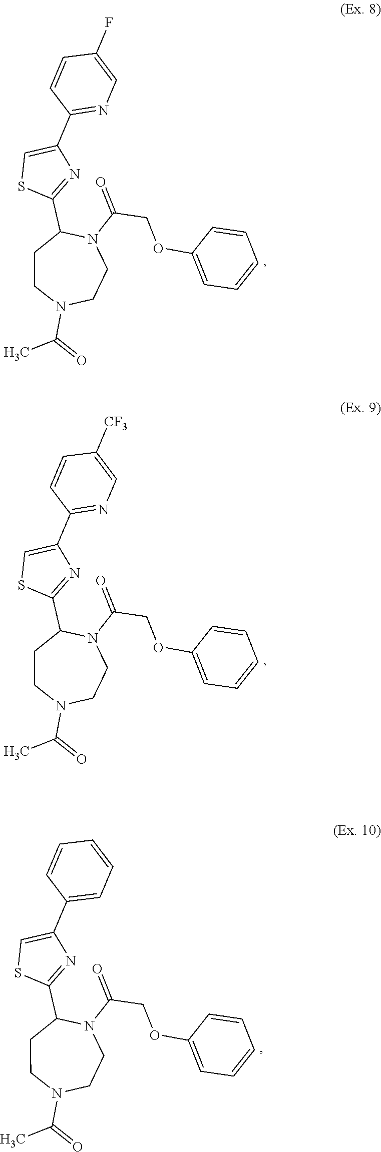 [SMILES: CC(=O)N1CCC(c2nc(-c3ccc(F)cn3)cs2)N(C(=O)COc2ccccc2)CC1 ; CC(=O)N1CCC(c2nc(-c3ccc(C(F)(F)F)cn3)cs2)N(C(=O)COc2ccccc2)CC1 ; CC(=O)N1CCC(c2nc(-c3ccccc3)cs2)N(C(=O)COc2ccccc2)CC1]

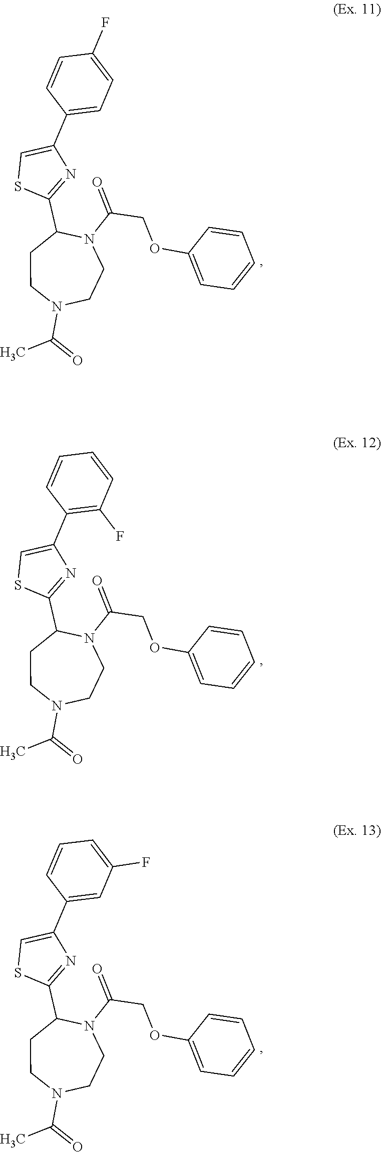 [SMILES: CC(=O)N1CCC(c2nc(-c3ccc(F)cc3)cs2)N(C(=O)COc2ccccc2)CC1 ; CC(=O)N1CCC(c2nc(-c3ccccc3F)cs2)N(C(=O)COc2ccccc2)CC1 ; CC(=O)N1CCC(c2nc(-c3cccc(F)c3)cs2)N(C(=O)COc2ccccc2)CC1]

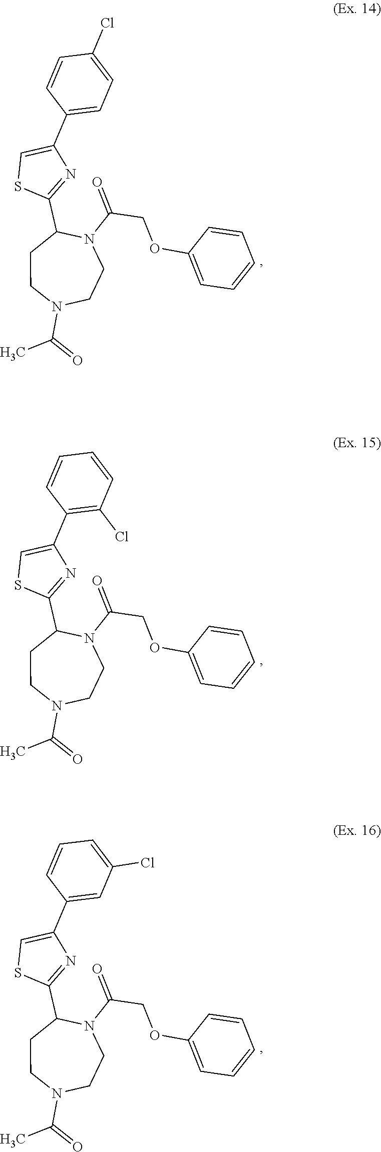 [SMILES: CC(=O)N1CCC(c2nc(-c3ccc(Cl)cc3)cs2)N(C(=O)COc2ccccc2)CC1 ; CC(=O)N1CCC(c2nc(-c3ccccc3Cl)cs2)N(C(=O)COc2ccccc2)CC1 ; CC(=O)N1CCC(c2nc(-c3cccc(Cl)c3)cs2)N(C(=O)COc2ccccc2)CC1]

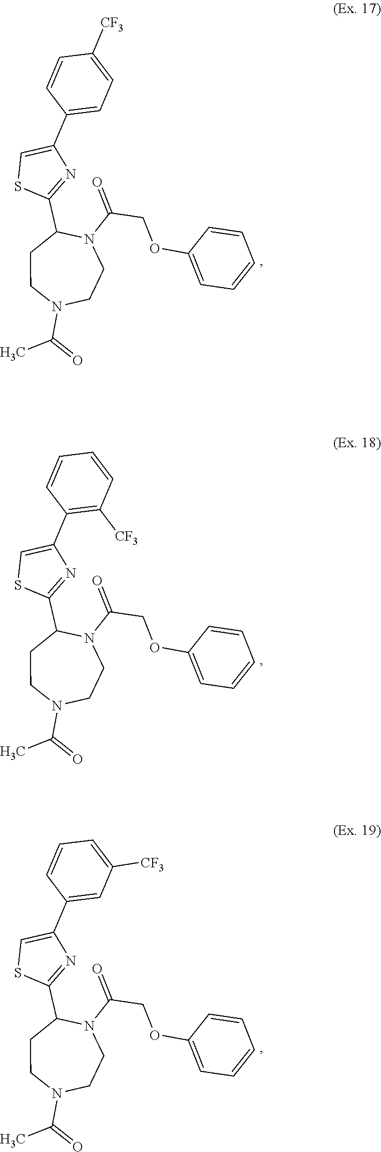 [SMILES: CC(=O)N1CCC(c2nc(-c3ccc(C(F)(F)F)cc3)cs2)N(C(=O)COc2ccccc2)CC1 ; CC(=O)N1CCC(c2nc(-c3ccccc3C(F)(F)F)cs2)N(C(=O)COc2ccccc2)CC1 ; CC(=O)N1CCC(c2nc(-c3cccc(C(F)(F)F)c3)cs2)N(C(=O)COc2ccccc2)CC1]

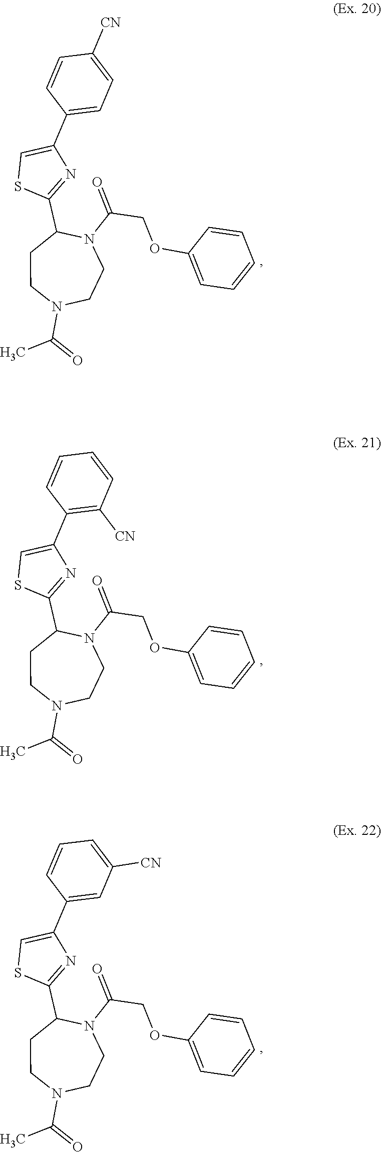 [SMILES: CC(=O)N1CCC(c2nc(-c3ccc(C#N)cc3)cs2)N(C(=O)COc2ccccc2)CC1 ; CC(=O)N1CCC(c2nc(-c3ccccc3C#N)cs2)N(C(=O)COc2ccccc2)CC1 ; CC(=O)N1CCC(c2nc(-c3cccc(C#N)c3)cs2)N(C(=O)COc2ccccc2)CC1]

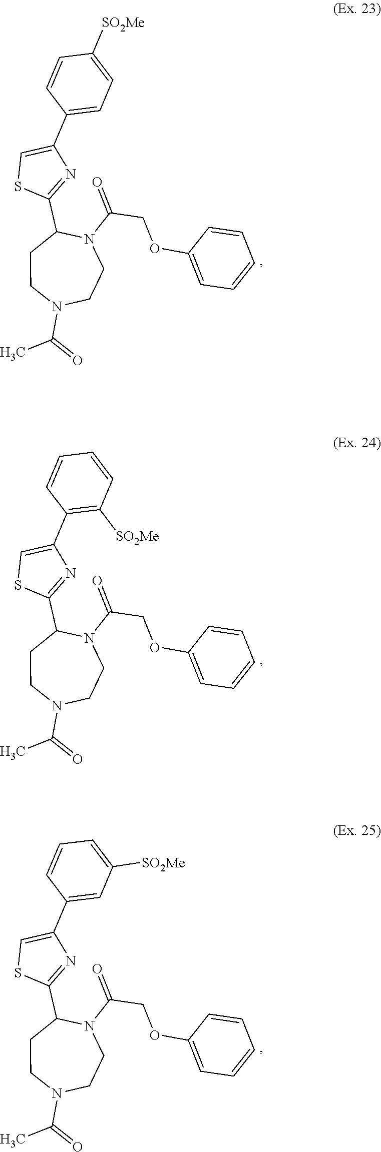 [SMILES: CC(=O)N1CCC(c2nc(-c3ccc(S(C)(=O)=O)cc3)cs2)N(C(=O)COc2ccccc2)CC1 ; CC(=O)N1CCC(c2nc(-c3ccccc3S(C)(=O)=O)cs2)N(C(=O)COc2ccccc2)CC1 ; CC(=O)N1CCC(c2nc(-c3cccc(S(C)(=O)=O)c3)cs2)N(C(=O)COc2ccccc2)CC1]

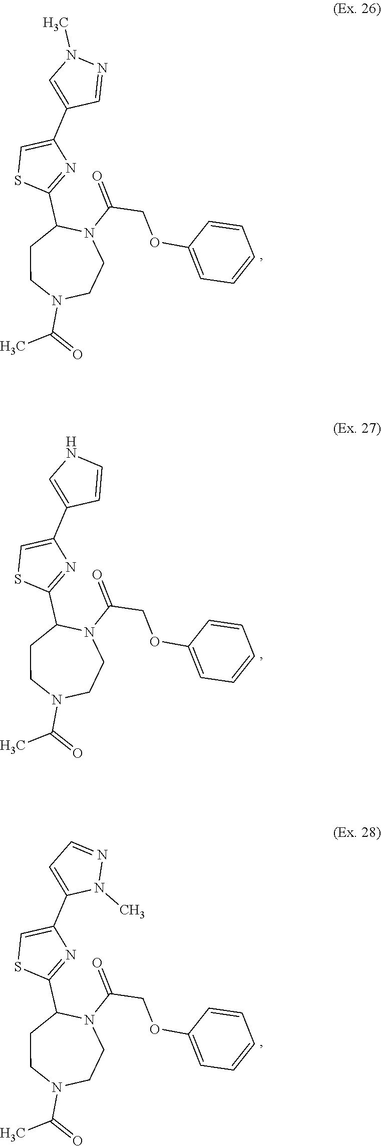 [SMILES: CC(=O)N1CCC(c2nc(-c3cnn(C)c3)cs2)N(C(=O)COc2ccccc2)CC1 ; CC(=O)N1CCC(c2nc(-c3cc[nH]c3)cs2)N(C(=O)COc2ccccc2)CC1 ; CC(=O)N1CCC(c2nc(-c3ccnn3C)cs2)N(C(=O)COc2ccccc2)CC1]

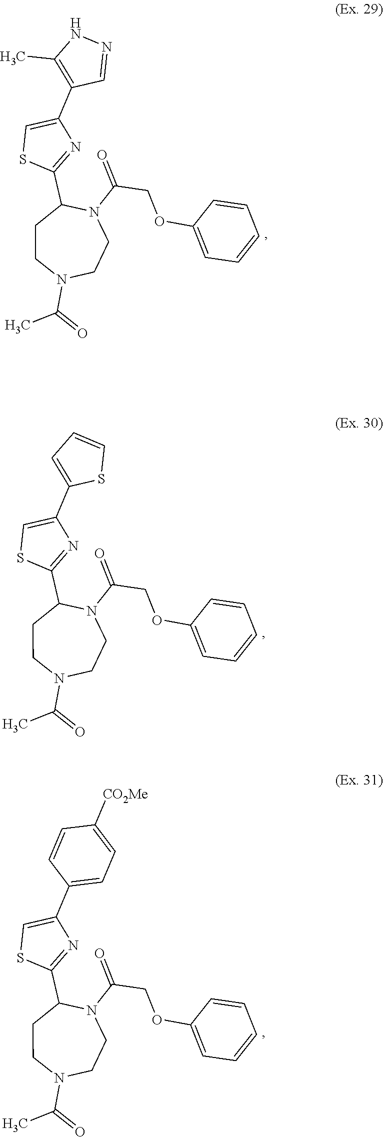 [SMILES: CC(=O)N1CCC(c2nc(-c3cn[nH]c3C)cs2)N(C(=O)COc2ccccc2)CC1 ; CC(=O)N1CCC(c2nc(-c3cccs3)cs2)N(C(=O)COc2ccccc2)CC1 ; COC(=O)c1ccc(-c2csc(C3CCN(C(C)=O)CCN3C(=O)COc3ccccc3)n2)cc1]

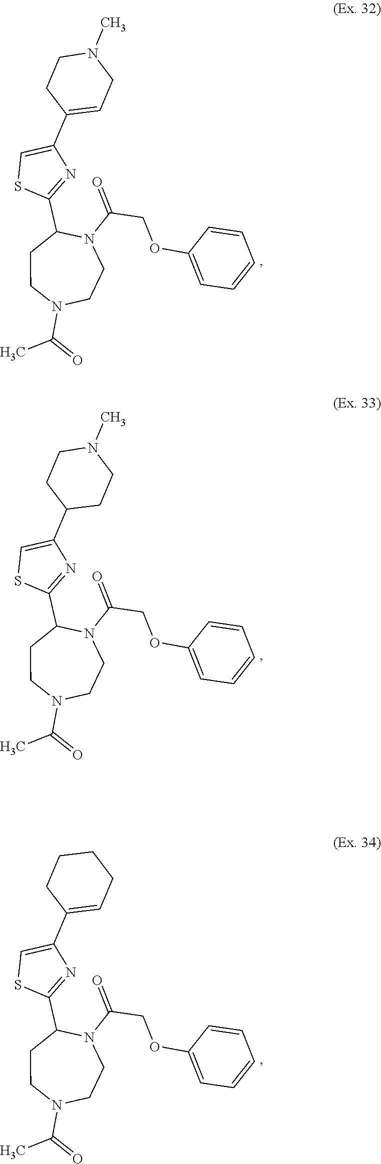 [SMILES: CC(=O)N1CCC(c2nc(C3=CCN(C)CC3)cs2)N(C(=O)COc2ccccc2)CC1 ; CC(=O)N1CCC(c2nc(C3CCN(C)CC3)cs2)N(C(=O)COc2ccccc2)CC1 ; CC(=O)N1CCC(c2nc(C3=CCCCC3)cs2)N(C(=O)COc2ccccc2)CC1]

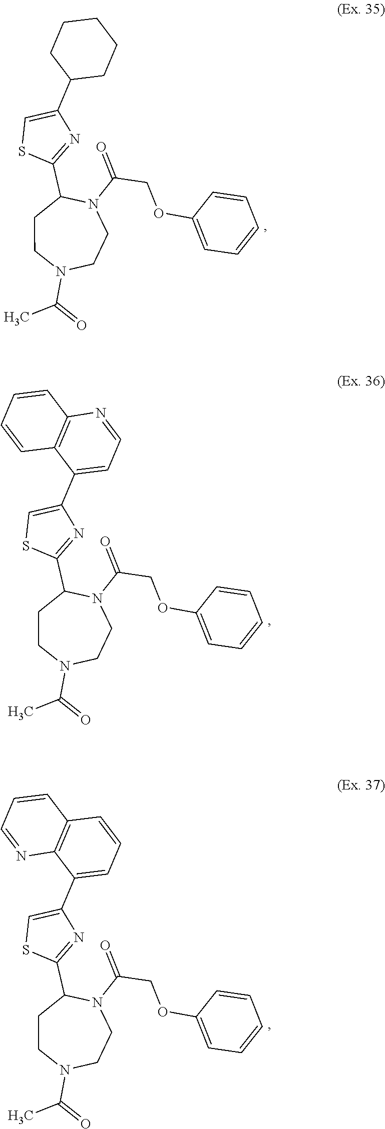 [SMILES: CC(=O)N1CCC(c2nc(C3CCCCC3)cs2)N(C(=O)COc2ccccc2)CC1 ; CC(=O)N1CCC(c2nc(-c3ccnc4ccccc34)cs2)N(C(=O)COc2ccccc2)CC1 ; CC(=O)N1CCC(c2nc(-c3cccc4cccnc34)cs2)N(C(=O)COc2ccccc2)CC1]

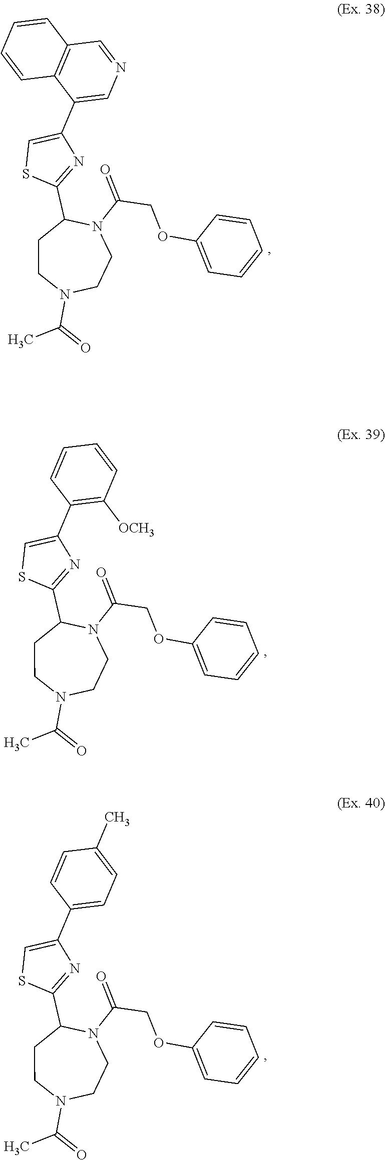 [SMILES: CC(=O)N1CCC(c2nc(-c3cncc4ccccc34)cs2)N(C(=O)COc2ccccc2)CC1 ; COc1ccccc1-c1csc(C2CCN(C(C)=O)CCN2C(=O)COc2ccccc2)n1 ; CC(=O)N1CCC(c2nc(-c3ccc(C)cc3)cs2)N(C(=O)COc2ccccc2)CC1]

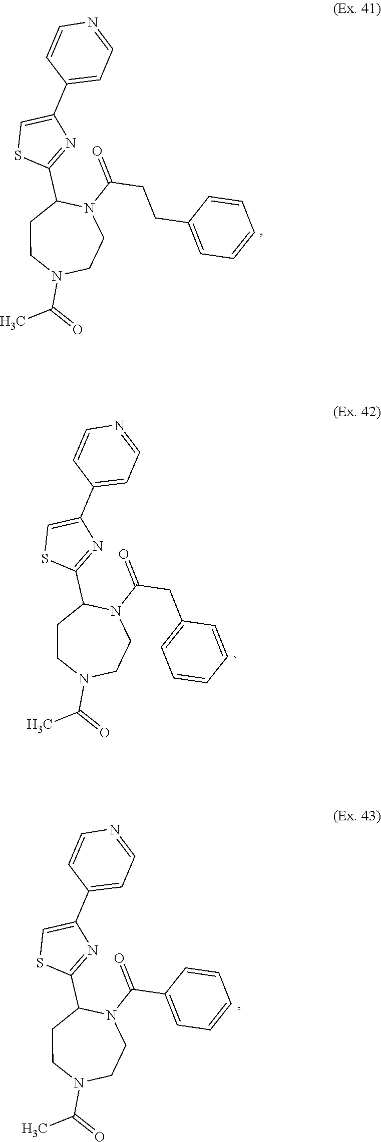 [SMILES: CC(=O)N1CCC(c2nc(-c3ccncc3)cs2)N(C(=O)CCc2ccccc2)CC1 ; CC(=O)N1CCC(c2nc(-c3ccncc3)cs2)N(C(=O)Cc2ccccc2)CC1 ; CC(=O)N1CCC(c2nc(-c3ccncc3)cs2)N(C(=O)c2ccccc2)CC1]

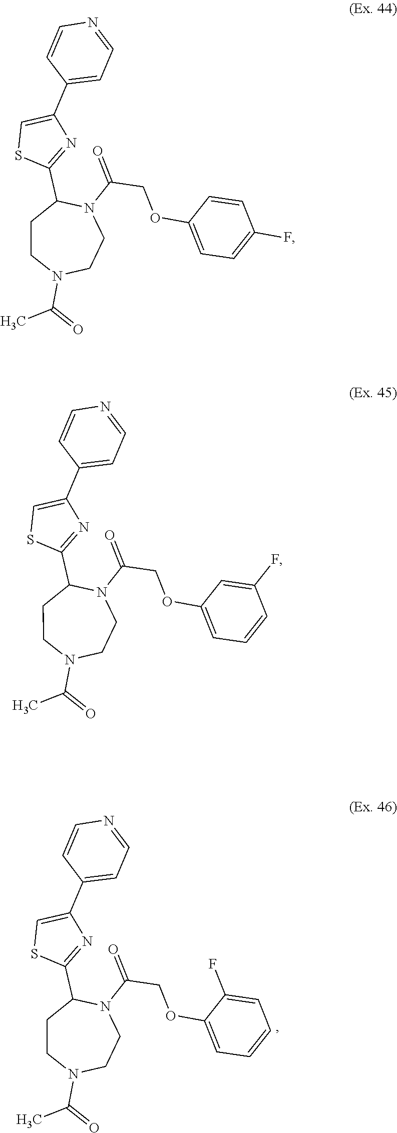 [SMILES: CC(=O)N1CCC(c2nc(-c3ccncc3)cs2)N(C(=O)COc2ccc(F)cc2)CC1 ; CC(=O)N1CCC(c2nc(-c3ccncc3)cs2)N(C(=O)COc2cccc(F)c2)CC1 ; CC(=O)N1CCC(c2nc(-c3ccncc3)cs2)N(C(=O)COc2ccccc2F)CC1]

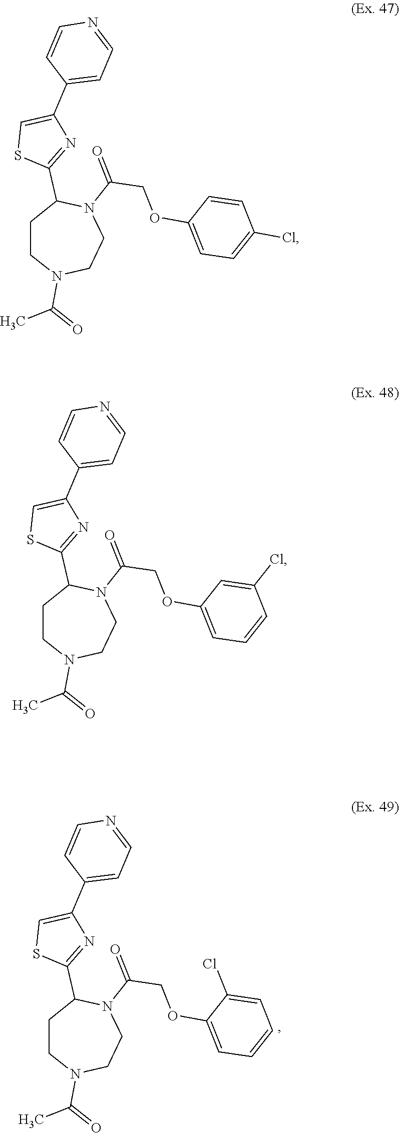 [SMILES: CC(=O)N1CCC(c2nc(-c3ccncc3)cs2)N(C(=O)COc2ccc(Cl)cc2)CC1 ; CC(=O)N1CCC(c2nc(-c3ccncc3)cs2)N(C(=O)COc2cccc(Cl)c2)CC1 ; CC(=O)N1CCC(c2nc(-c3ccncc3)cs2)N(C(=O)COc2ccccc2Cl)CC1]

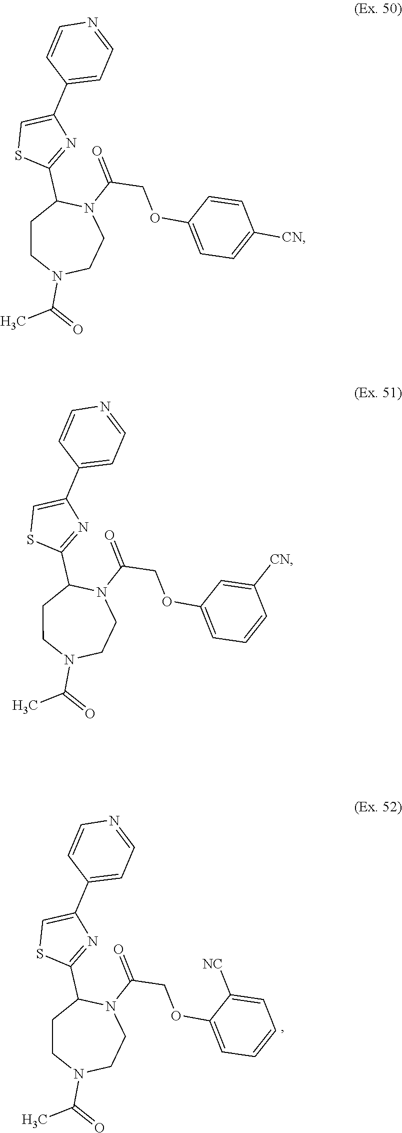 [SMILES: CC(=O)N1CCC(c2nc(-c3ccncc3)cs2)N(C(=O)COc2ccc(C#N)cc2)CC1 ; CC(=O)N1CCC(c2nc(-c3ccncc3)cs2)N(C(=O)COc2cccc(C#N)c2)CC1 ; CC(=O)N1CCC(c2nc(-c3ccncc3)cs2)N(C(=O)COc2ccccc2C#N)CC1]

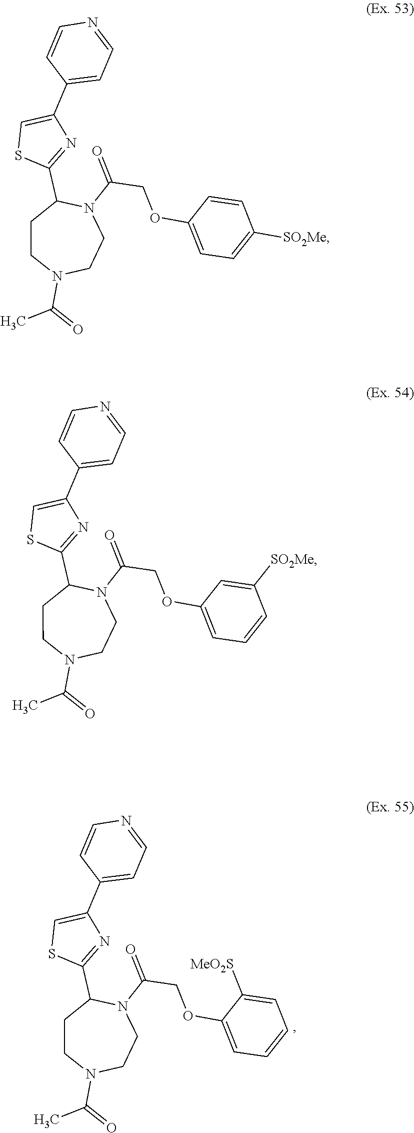 [SMILES: CC(=O)N1CCC(c2nc(-c3ccncc3)cs2)N(C(=O)COc2ccc(S(C)(=O)=O)cc2)CC1 ; CC(=O)N1CCC(c2nc(-c3ccncc3)cs2)N(C(=O)COc2cccc(S(C)(=O)=O)c2)CC1 ; CC(=O)N1CCC(c2nc(-c3ccncc3)cs2)N(C(=O)COc2ccccc2S(C)(=O)=O)CC1]

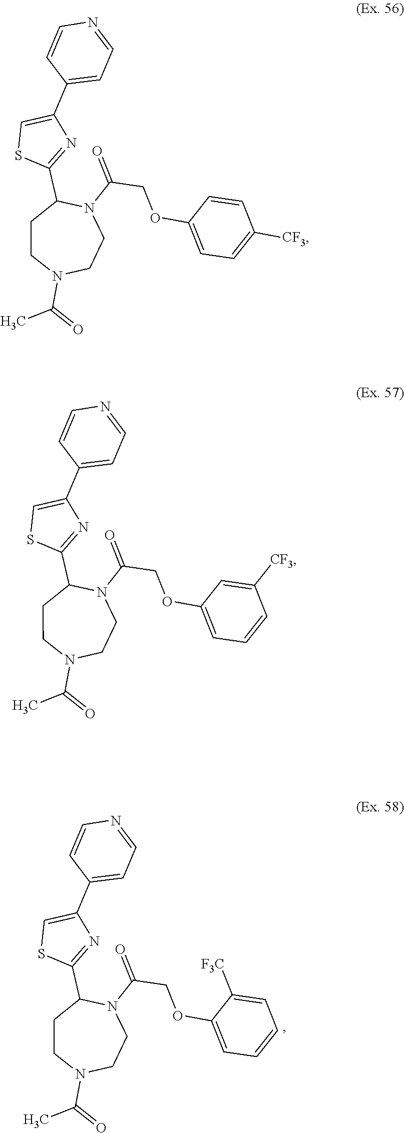 [SMILES: CC(=O)N1CCC(c2nc(-c3ccncc3)cs2)N(C(=O)COc2ccc(C(F)(F)F)cc2)CC1 ; CC(=O)N1CCC(c2nc(-c3ccncc3)cs2)N(C(=O)COc2cccc(C(F)(F)F)c2)CC1 ; CC(=O)N1CCC(c2nc(-c3ccncc3)cs2)N(C(=O)COc2ccccc2C(F)(F)F)CC1]

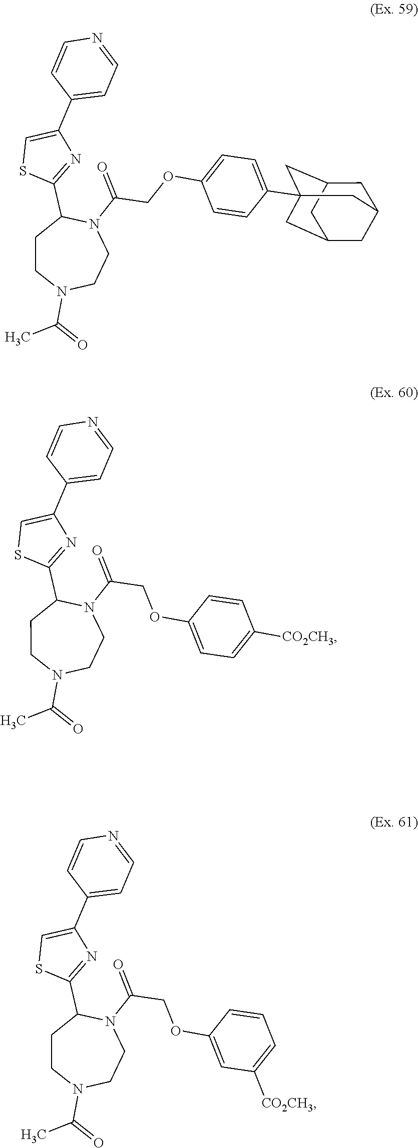 [SMILES: CC(=O)N1CCC(c2nc(-c3ccncc3)cs2)N(C(=O)COc2ccc(C34CC5CC(CC(C5)C3)C4)cc2)CC1 ; COC(=O)c1ccc(OCC(=O)N2CCN(C(C)=O)CCC2c2nc(-c3ccncc3)cs2)cc1 ; COC(=O)c1cccc(OCC(=O)N2CCN(C(C)=O)CCC2c2nc(-c3ccncc3)cs2)c1]

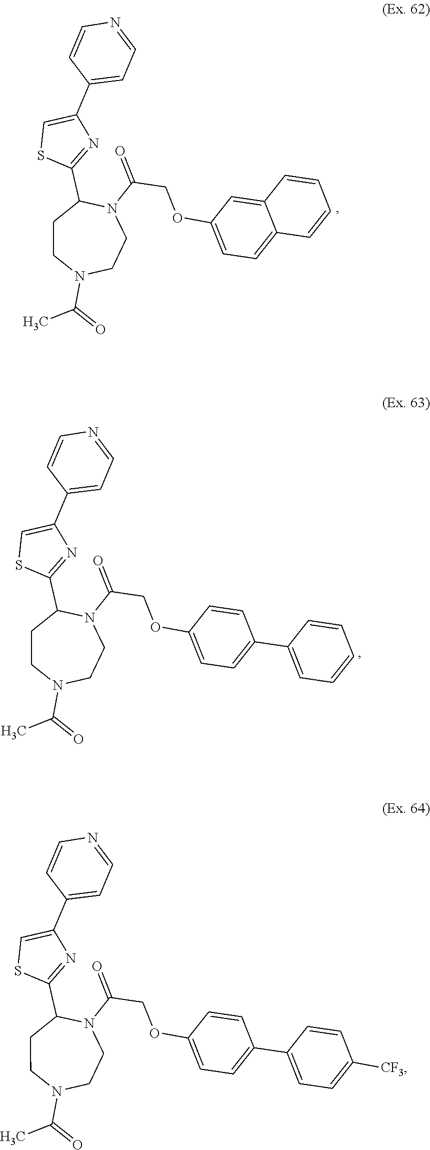 [SMILES: CC(=O)N1CCC(c2nc(-c3ccncc3)cs2)N(C(=O)COc2ccc3ccccc3c2)CC1 ; CC(=O)N1CCC(c2nc(-c3ccncc3)cs2)N(C(=O)COc2ccc(-c3ccccc3)cc2)CC1 ; CC(=O)N1CCC(c2nc(-c3ccncc3)cs2)N(C(=O)COc2ccc(-c3ccc(C(F)(F)F)cc3)cc2)CC1]

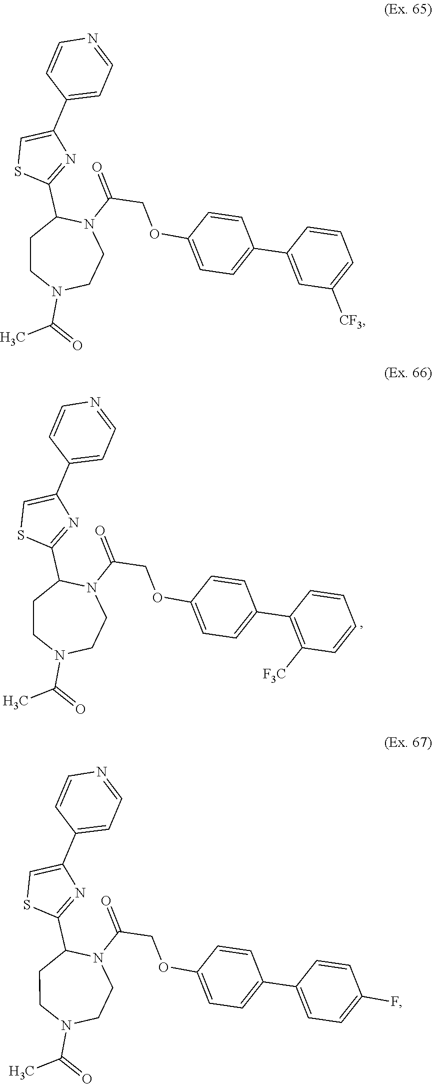 [SMILES: CC(=O)N1CCC(c2nc(-c3ccncc3)cs2)N(C(=O)COc2ccc(-c3cccc(C(F)(F)F)c3)cc2)CC1 ; CC(=O)N1CCC(c2nc(-c3ccncc3)cs2)N(C(=O)COc2ccc(-c3ccccc3C(F)(F)F)cc2)CC1 ; CC(=O)N1CCC(c2nc(-c3ccncc3)cs2)N(C(=O)COc2ccc(-c3ccc(F)cc3)cc2)CC1]

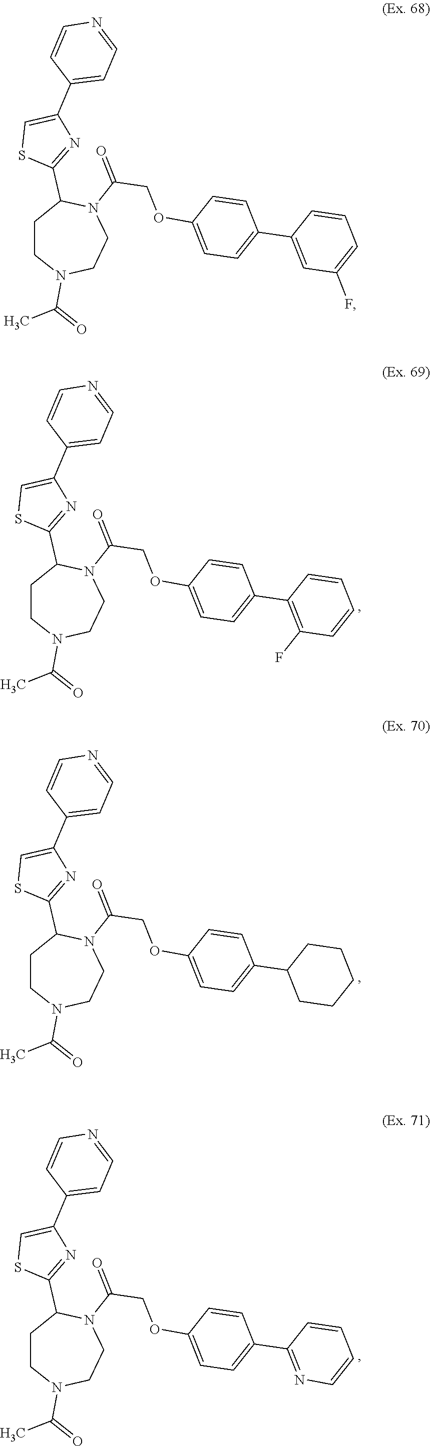 [SMILES: CC(=O)N1CCC(c2nc(-c3ccncc3)cs2)N(C(=O)COc2ccc(-c3cccc(F)c3)cc2)CC1 ; CC(=O)N1CCC(c2nc(-c3ccncc3)cs2)N(C(=O)COc2ccc(-c3ccccc3F)cc2)CC1 ; CC(=O)N1CCC(c2nc(-c3ccncc3)cs2)N(C(=O)COc2ccc(C3CCCCC3)cc2)CC1 ; CC(=O)N1CCC(c2nc(-c3ccncc3)cs2)N(C(=O)COc2ccc(-c3ccccn3)cc2)CC1]

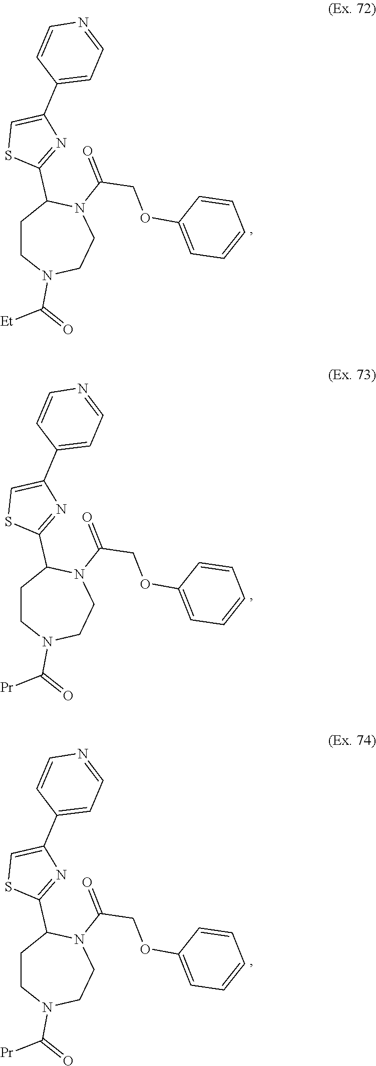

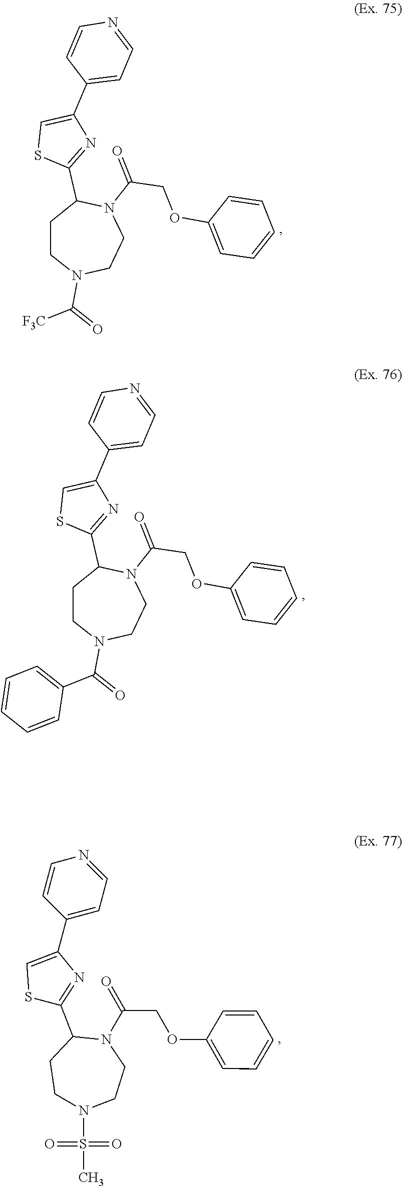 [SMILES: O=C(COc1ccccc1)N1CCN(C(=O)C(F)(F)F)CCC1c1nc(-c2ccncc2)cs1 ; O=C(c1ccccc1)N1CCC(c2nc(-c3ccncc3)cs2)N(C(=O)COc2ccccc2)CC1 ; CS(=O)(=O)N1CCC(c2nc(-c3ccncc3)cs2)N(C(=O)COc2ccccc2)CC1]

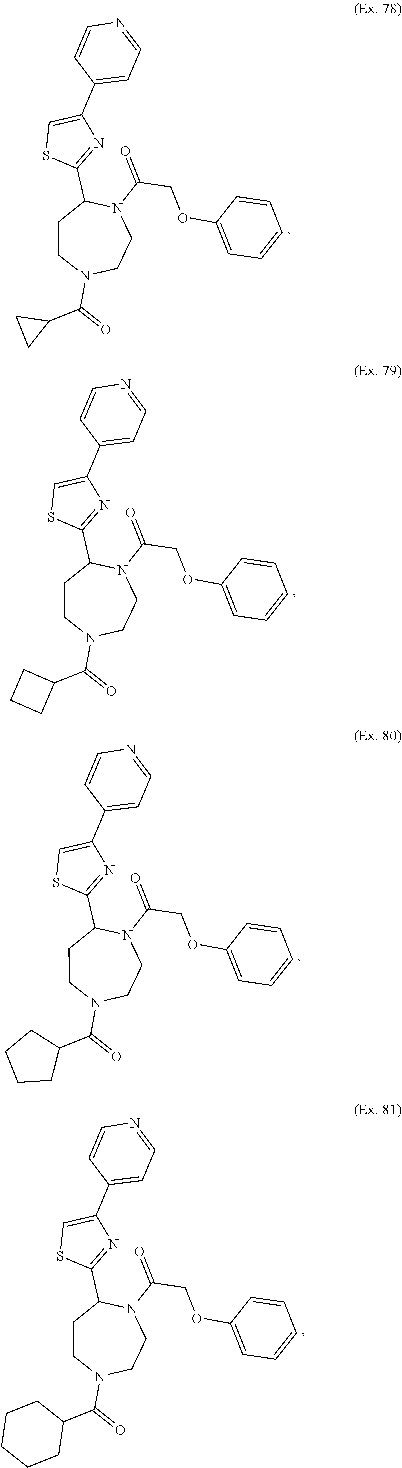 [SMILES: O=C(C1CC1)N1CCC(c2nc(-c3ccncc3)cs2)N(C(=O)COc2ccccc2)CC1 ; O=C(C1CCC1)N1CCC(c2nc(-c3ccncc3)cs2)N(C(=O)COc2ccccc2)CC1 ; O=C(C1CCCC1)N1CCC(c2nc(-c3ccncc3)cs2)N(C(=O)COc2ccccc2)CC1 ; O=C(C1CCCCC1)N1CCC(c2nc(-c3ccncc3)cs2)N(C(=O)COc2ccccc2)CC1]

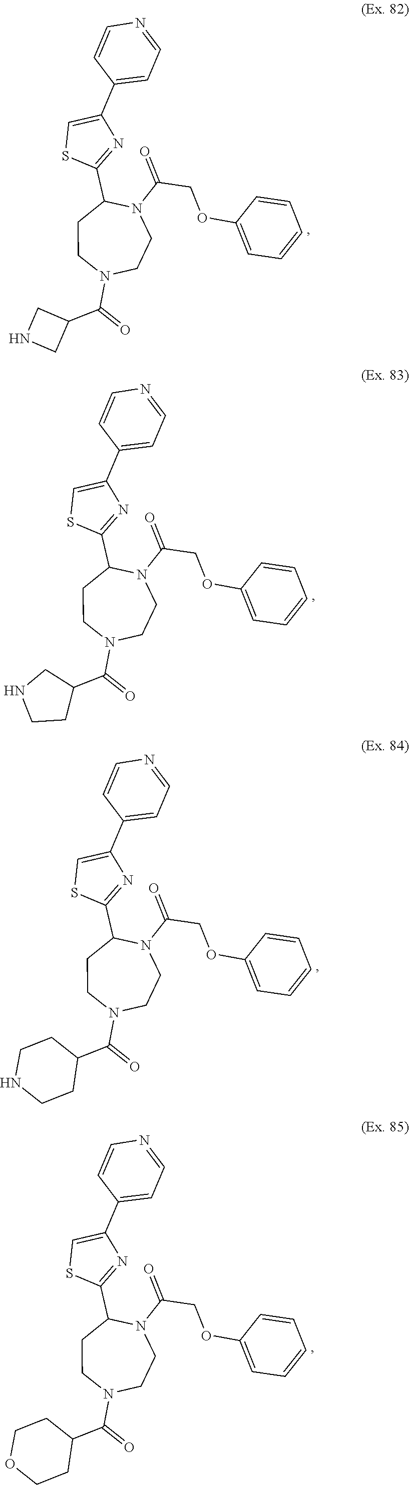 [SMILES: O=C(C1CNC1)N1CCC(c2nc(-c3ccncc3)cs2)N(C(=O)COc2ccccc2)CC1 ; O=C(C1CCNC1)N1CCC(c2nc(-c3ccncc3)cs2)N(C(=O)COc2ccccc2)CC1 ; O=C(C1CCNCC1)N1CCC(c2nc(-c3ccncc3)cs2)N(C(=O)COc2ccccc2)CC1 ; O=C(C1CCOCC1)N1CCC(c2nc(-c3ccncc3)cs2)N(C(=O)COc2ccccc2)CC1]

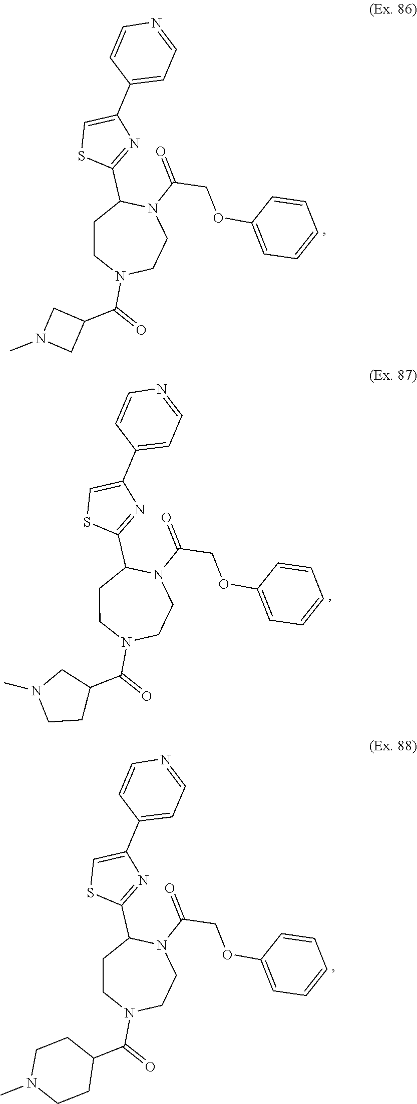 [SMILES: CN1CC(C(=O)N2CCC(c3nc(-c4ccncc4)cs3)N(C(=O)COc3ccccc3)CC2)C1 ; CN1CCC(C(=O)N2CCC(c3nc(-c4ccncc4)cs3)N(C(=O)COc3ccccc3)CC2)C1 ; CN1CCC(C(=O)N2CCC(c3nc(-c4ccncc4)cs3)N(C(=O)COc3ccccc3)CC2)CC1]

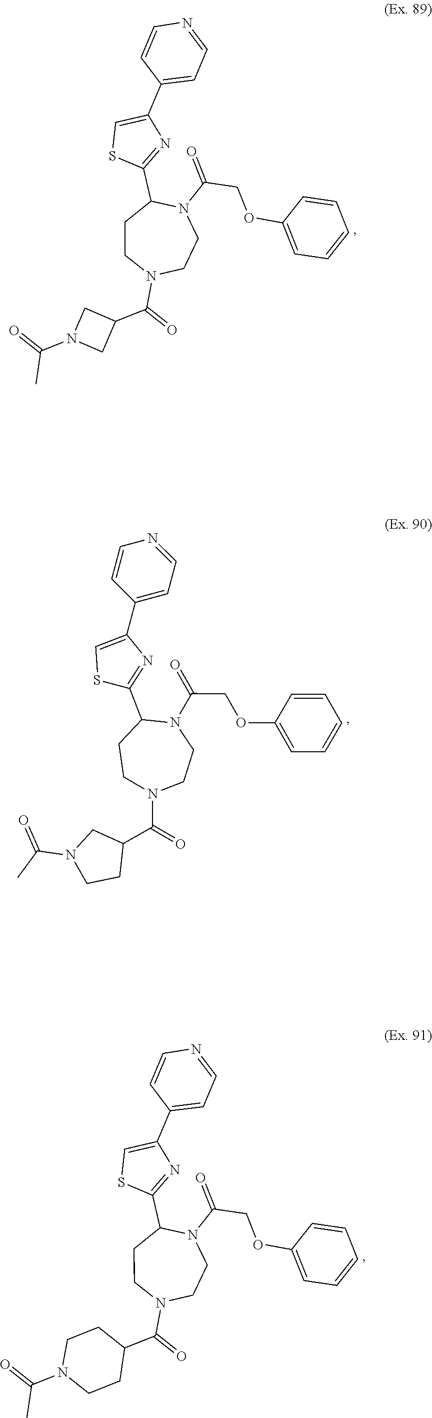 [SMILES: CC(=O)N1CC(C(=O)N2CCC(c3nc(-c4ccncc4)cs3)N(C(=O)COc3ccccc3)CC2)C1 ; CC(=O)N1CCC(C(=O)N2CCC(c3nc(-c4ccncc4)cs3)N(C(=O)COc3ccccc3)CC2)C1 ; CC(=O)N1CCC(C(=O)N2CCC(c3nc(-c4ccncc4)cs3)N(C(=O)COc3ccccc3)CC2)CC1]

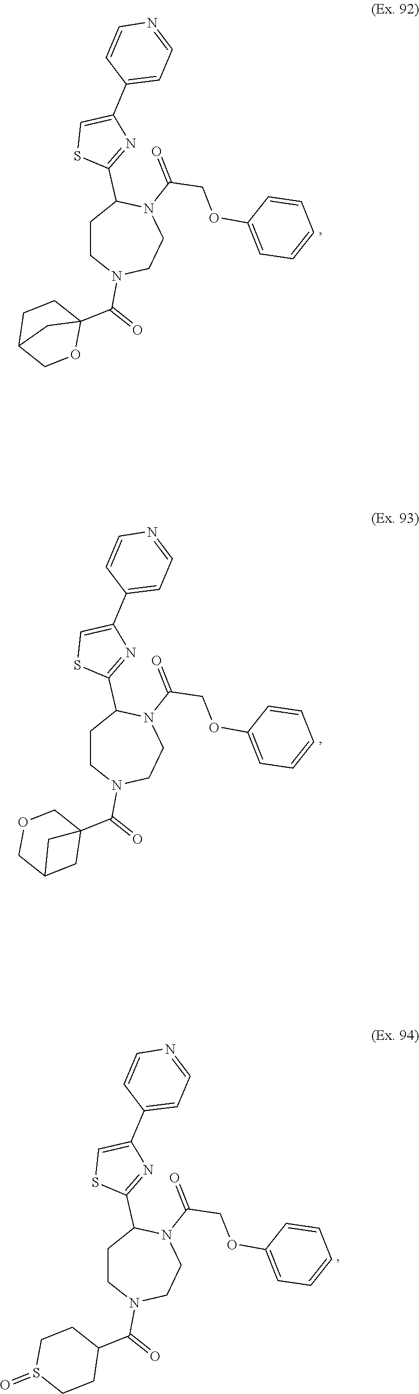 [SMILES: O=C(COc1ccccc1)N1CCN(C(=O)C23CCC(CO2)C3)CCC1c1nc(-c2ccncc2)cs1 ; O=C(COc1ccccc1)N1CCN(C(=O)C23COCC(C2)C3)CCC1c1nc(-c2ccncc2)cs1 ; O=C(C1CCS(=O)CC1)N1CCC(c2nc(-c3ccncc3)cs2)N(C(=O)COc2ccccc2)CC1]

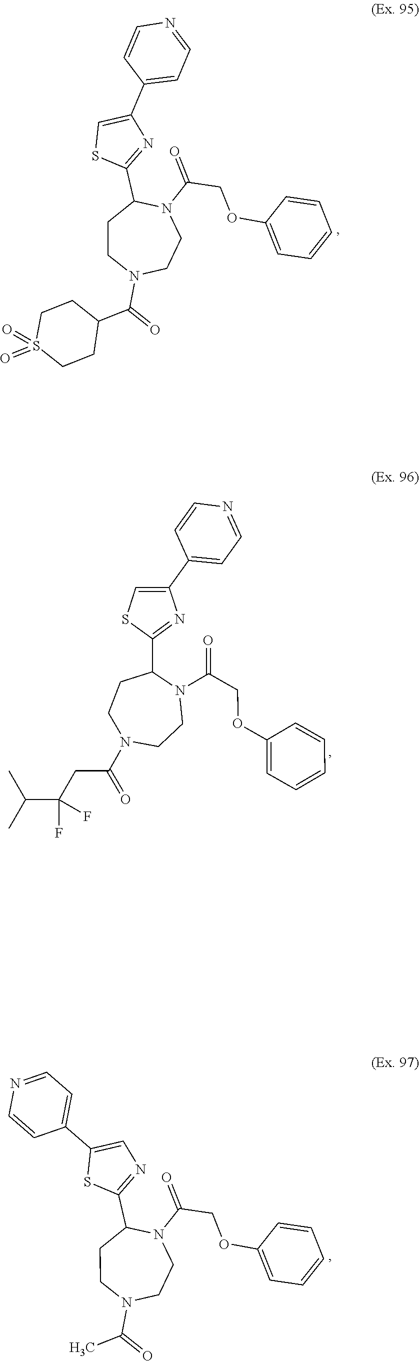 [SMILES: O=C(C1CCS(=O)(=O)CC1)N1CCC(c2nc(-c3ccncc3)cs2)N(C(=O)COc2ccccc2)CC1 ; CC(C)C(F)(F)CC(=O)N1CCC(c2nc(-c3ccncc3)cs2)N(C(=O)COc2ccccc2)CC1 ; CC(=O)N1CCC(c2ncc(-c3ccncc3)s2)N(C(=O)COc2ccccc2)CC1]

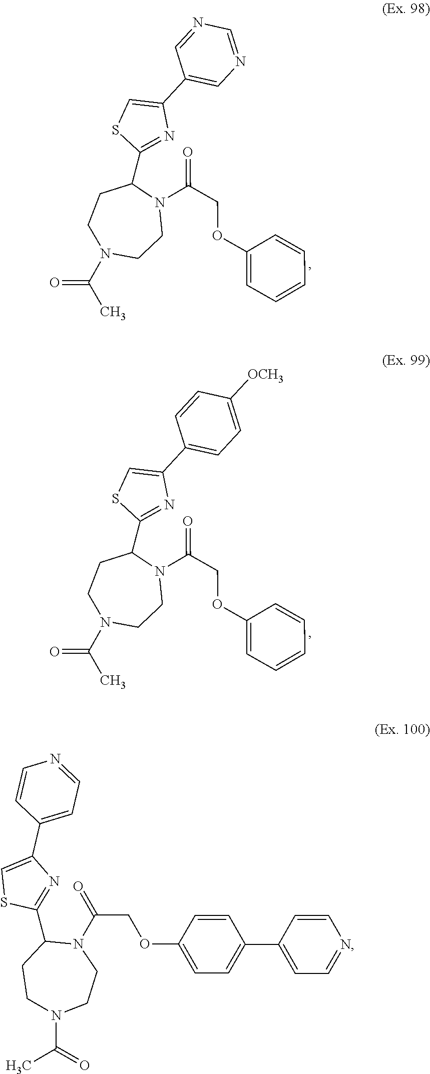 [SMILES: CC(=O)N1CCC(c2nc(-c3cncnc3)cs2)N(C(=O)COc2ccccc2)CC1 ; COc1ccc(-c2csc(C3CCN(C(C)=O)CCN3C(=O)COc3ccccc3)n2)cc1 ; CC(=O)N1CCC(c2nc(-c3ccncc3)cs2)N(C(=O)COc2ccc(-c3ccncc3)cc2)CC1]

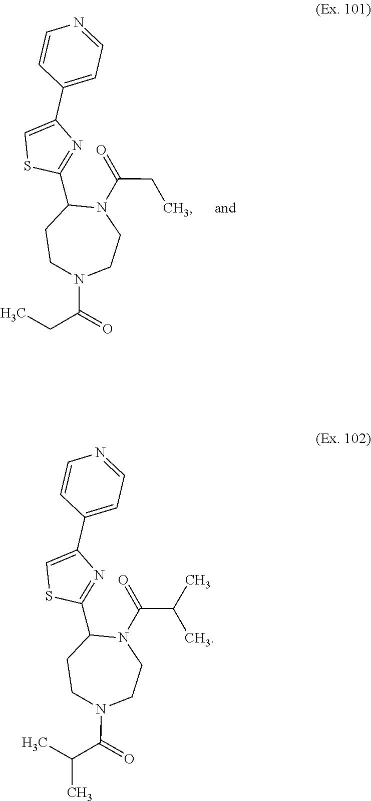 [SMILES: CCC(=O)N1CCC(c2nc(-c3ccncc3)cs2)N(C(=O)CC)CC1 ; "and" ; CC(C)C(=O)N1CCC(c2nc(-c3ccncc3)cs2)N(C(=O)C(C)C)CC1]

In another aspect, the invention relates to a pharmaceutical composition comprising the Carnitine-Palmitoyl-Transferase-1 (CPT-1) inhibitor as defined herein for use as defined herein, further comprising a pharmaceutically acceptable excipient.

### BRIEF DESCRIPTION OF THE FIGURES

FIG. 1 shows the efficacy of the CPT-1 inhibitors, Example 1 (Ex. 1, racemic mixture), tested in the fatty acid uptake assay using HEK293 cells with IC₅₀ of 0.3 μM.

FIG. 2 shows the efficacy of the CPT-1 inhibitor of Example 1 (Ex. 1, racemic mixture) in the CLP-induced mouse model of sepsis.

### DETAILED DESCRIPTION OF THE INVENTION

### Definitions

As used herein, including the accompanying claims, the substituents and terms, which are collectively used, have the following meanings.

As used herein, the term “subject” refers to an animal, especially a mammal. Non-limiting examples of mammals include humans, non-human primates, dogs, cats, equines, bovines, and pigs. The preferred subject in the context of this invention, however, is a human of any gender and of any age or stage of development, including infant, toddler, child, adolescent, teenager, adult and senior.

As used herein, the term “preventing” a condition or disorder refers to avoiding, delaying, forestalling, or reducing the onset of a particular sign or symptom of the condition or disorder. Prevention can, but is not required to be, absolute or complete; meaning the sign or symptom may still develop at a later time. Prevention can include reducing the severity of the onset of such a condition or disorder, and/or inhibiting the progression of the condition or disorder to a more severe condition or disorder.

As used herein, the term “treatment” refers to eradicating, reducing, ameliorating, or reversing, a sign or symptom of a condition or disorder to any extent, and includes, but does not require, a complete cure of the condition or disorder. Treating can be curing, improving, or partially ameliorating a disorder.

The term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of limits the scope of a claim to the specified materials or steps” and those that do not materially affect the basic and novel characteristic(s)” of the claimed invention. Use of the term “comprising” contemplates other embodiments that “consist” or “consist essentially” of the recited component(s).

The terms “about”, “approximately”, “approximate,” and “around” are used in this patent application to describe some quantitative aspects of the invention, for example, the concentration of the active agent. It should be understood that absolute accuracy is not required with respect to those aspects for the invention to operate. When these terms are used to describe a quantitative aspect of the invention, the relevant aspect may be varied by up to ±10%. Thus, the terms “about”, “approximately”, “approximate” and “around” allow for variation of the various disclosed quantitative aspects of the invention by ±1%, ±2%, ±3%, ±4%, ±5%, ±6%, ±7%, ±8%, ±9%, or up to ±10%. For example, a composition comprising about 1% active agent can contain 0.9% to 1.1% active agent.

As used herein, the term “aryl” means a mono-, bi- or polycyclic aromatic system, for example unsubstituted or substituted phenyl, naphthyl, tetrahydronaphthyl, indenyl, indanyl, pentalenyl, fluorenyl and the like, preferably unsubstituted or substituted phenyl and naphthyl, particularly preferred unsubstituted or substituted phenyl.

As used herein, the term “heteroaryl” means an aromatic or partly unsaturated 5- or 6-membered ring which comprises one, two or three atoms selected from nitrogen, oxygen and/or sulphur, such as pyridyl, pyrazinyl, pyrimidinyl, pyridazinyl, 2-oxo-1,2-dihydropyridinyl, oxazolyl, oxydiazolyl, isoxazolyl, thiadiazolyl, tetrazolyl, pyrazolyl, imidazolyl, thiazolyl and thienyl. The term “heteroaryl” further refers to bicyclic aromatic or partly unsaturated groups comprising two 5- or 6-membered rings, in which one or both rings can contain one, two or three atoms selected from nitrogen, oxygen or sulphur, such as quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, indazolyl. Preferred heteroaryl groups are pyridyl, pyrazolyl, thienyl and pyrazinyl.

As used herein, the term “4-, 5- or 6-membered saturated or partially unsaturated heterocyclyl” represents an unsubstituted or substituted saturated or partially unsaturated ring system containing 4, 5 or 6 ring atoms and containing in addition to C ring atoms one to three nitrogen atoms and/or an oxygen or sulfur atom or one or two oxygen and/or sulfur atoms. In a particular preferred embodiment the “4-, 5- or 6-membered saturated heterocyclyl” represents an unsubstituted or substituted saturated ring system containing 4, 5 or 6 ring atoms and containing in addition to C ring atoms one to three nitrogen atoms and/or an oxygen or sulfur atom or one or two oxygen and/or sulfur atoms.

In a preferred embodiment, the 4-, 5- or 6-membered saturated heterocyclyl contains in addition to C ring atoms one N and optionally one additional heteroatom. The additional heteroatoms are preferably selected from O, N or S. Especially preferred are heterocycles with only one N as a heteroatom. Preferably, these substituted heterocycles are single or twofold substituted. The 4-, 5- or 6-membered saturated heterocycle may be substituted at the C atom(s), at the O atom(s), at the N atom(s) or at the S atom(s). Examples of 4-, 5- or 6-membered saturated heterocyclyl include, but are not limited to oxetanyl, azetidinyl, 1,3-diazetinyl, thietanyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2,3-pyrrolin-2-yl, 2,3-pyrrolin-3-yl, 2,4-pyrrolin-2-yl, 2,4-pyrrolin-3-yl, 2,3-isoxazolin-3-yl, 3,4-isoxazolin-3-yl, 4,5-isoxazolin-3-yl, 2,3-isoxazolin-4-yl, 3,4-isoxazolin-4-yl, 4,5-isoxazolin-4-yl, 2,3-isoxazolin-5-yl, 3,4-isoxazolin-5-yl, 4,5-isoxazolin-5-yl, 2,3-isothiazolin-3-yl, 3,4-isothiazolin-3-yl, 4,5-isothiazolin-3-yl, 2,3-isothiazolin-4-yl, 3,4-isothiazolin-4-yl, 4,5-isothiazolin-4-yl, 2,3-isothiazolin-5-yl, 3,4-isothiazolin-5-yl, 4,5-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1-piperazinyl, 2-piperazinyl, 1,3-dioxan-5-yl, 2-tetrahydropyranyl, 4-tetrahydropyranyl, 2-tetrahydrothienyl, 3-tetrahydropyridazinyl, 4-tetrahydropyridazinyl, 2-tetrahydropyrimidinyl, 4-tetrahydropyrimidinyl, 5-tetrahydropyrimidinyl, 2-tetrahydropyrazinyl, 1,3,5-tetrahydrotriazin-2-yl and 1,2,4-tetrahydrotriazin-3-yl, preferably piperidin-1-yl, 2-piperidinyl, 3-piperidinyl, 4-piperidinyl, 1-piperazinyl, 2-piperazinyl, 2-pyrrolidinyl, and 3-pyrrolidinyl, tetrahydropyridinyl, preferably 1,2,3,6-tetrahydropyridinyl, 1,2-oxazinyl, 1,3-oxazinyl, and 1,4-oxazinyl.

As used herein, the term “C₃-C₈-cycloalkyl” means a carbocyclic saturated ring system having 3 to 8 carbon atoms, preferably 3 to 6, particularly preferred 5 to 6 carbon atoms, for example cyclopropyl, cyclobutyl, cyclopentyl, cyclohexyl, cycloheptyl or cyclooctyl, preferably cyclopentyl and cyclohexyl.

The aryl, heteroaryl, 4-, 5- or 6-membered saturated or partially unsaturated heterocyclyl or the C₃-C₈-cycloalkyl, may be each optionally and independently substituted with one or more, preferably with one of the following residues:

—CN,

halogen, preferably —F or —Cl,

C₁-C₄-alkyl, preferably methyl, halogen-C₁-C₄-alkyl, preferably difluoromethyl, or trifluoromethyl, SO₂Me, or CO₂C₁-C₄-alkyl, preferably CO₂Me.

As used herein, the term “C₁-C₄-alkyl” means a straight-chain or branched-chain alkyl group with 1 to 4 carbon atoms, respectively. Examples of straight-chain and branched groups include, but are not limited to, methyl, ethyl, propyl, isopropyl, butyl, isobutyl, and tert-butyl, preferably methyl and ethyl and most preferred methyl.

As used herein, the term “halogen-C₁-C₄-alkyl” means a straight-chain or branched alkyl group having 1 to 4 carbon atoms (as mentioned above), it being possible for the hydrogen atoms in these groups to be partly or completely replaced by halogen atoms as mentioned above, e.g. C₁-C₂-halogenalkyl such as chloromethyl, dichloromethyl, trichloromethyl, fluoromethyl, difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2fluoroethyl, 2-chloro-2,2-difluoroethyl, 2,2-dichloro-2-fluoroethyl, 2,2,2-trichloroethyl and pentafluoroethyl, preferably trifluoromethyl.

As used herein, the term “substitution” or “substituted” means one or more substituents commonly known in the art, or as specifically defined herein.

As used herein, the term “halogen” represents fluoro, chloro, bromo or iodo, preferably represents fluoro and chloro.

As used herein, the term “stereoisomer(s)” as it relates to a compound of formula (I) and to its intermediate compounds means any possible enantiomers or diastereomers of a compound of formula (I) and its salts or hydrates. In particular, the term “stereoisomer” means a single compound or a mixture of two or more compounds, wherein at least one chiral center is predominantly present in one definite isomeric form, in particular the S-enantiomer, the R-enantiomer and the racemate of a compound of formula (I). It is also possible that two or more stereogenic centers are predominantly present in one definite isomeric form of a derivative of a compound of formula (I) as defined above. In the sense of the present invention, “predominantly” has the meaning of at least 60%, preferably at least 70%, particularly preferably at least 80%, most preferably at least 90%. According to the present invention, also stereoisomers of a compound of formula (I) may be present as a salt or a hydrate.

As used herein, the term “salt(s)” as it relates to a compound of formula (I) as defined above means the physiologically acceptable acid addition salts and base salts of the compound of formula (I), i.e. its pharmaceutically or veterinarily acceptable salts, or its derivatives or its stereoisomers. Suitable acid addition salts are formed from acids which form non-toxic salts. Examples include but are not limited to the acetate, aspartate, benzoate, besylate, bicarbonate, carbonate, bisulphate, sulphate, borate, camsylate, citrate, edisylate, esylate, formate, fumarate, gluceptate, gluconate, glucuronate, hexafluorophosphate, hibenzate, hydrochloride/chloride, hydrobromide, bromide, hydroiodide, iodide, isethionate, lactate, malate, maleate, malonate, mesylate, methylsulphate, naphthylate, nicotinate, nitrate, orotate, oxalate, palmitate, pamoate, phosphate, hydrogen phosphate, dihydrogen phosphate, sacharate, stearate, succinate, tartrate, tosylate and trifluoroacetate salts. Suitable base salts are formed from bases which form non-toxic salts. Examples include but are not limited to the aluminium, arginine, benzathine, calcium, choline, diethylamine, diolamine, glycine, lysine, magnesium, meglumine, olamine, potassium, sodium, tromethamine and zinc salts.

As used herein, the term “hydrate(s)” as it relates to a compound of formula (I) means a compound of formula (I) or a stereoisomer or a salt thereof that includes water. “Hydrate(s)” are formed by the addition of water or its elements. In one embodiment, a compound of formula (I) as defined above or a stereoisomer or a salt thereof may form crystals that incorporate water into the crystalline structure without chemical alteration.

The terms stereoisomer, salt, and hydrate may also be used in conjunction with one another. For example, a stereoisomer of a compound of formula (I) may have a salt. Combinations of these terms are considered to be within the scope of the invention.

Technical terms are used by their common sense. If a specific meaning is conveyed to certain terms, definitions of terms will be given in the following in the context of which the terms are used.

The below mentioned general or preferred residue definitions apply both to the end products of the formula (I) and to specific embodiments thereof, and also, correspondingly, to the starting materials or to intermediates of formulae (A to (G) required in each case for the preparation. These residue definitions can be combined with one another at will, i.e. including combinations between the given preferred residues. Further, individual definitions may not apply.

As used herein, the terms “CPT-I inhibitor” or “inhibiting agent” mean any compound capable of down-regulating, decreasing, reducing, suppressing, or inactivating the amount and/or activity of the enzyme Carnitine-Palmitoyl-Transferase-1 (CPT-I), which is a key enzyme of the fatty acid oxidation pathway, and has the following catalytic activity: Palmitoyl-CoA+L-carnitine=CoA+L-palmitoylcarnitine. The enzyme is also known under the following synonyms: EC 2.3.1.21, CPT I, CPTI-L, Carnitine palmitoyltransferase IA, Carnitine palmitoyltransferase IB, Carnitine palmitoyltransferase 1C, CPT IM. Generally, CPT-I inhibitors or inhibiting agents may be proteins, oligo- and polypeptides, nucleic acids, genes, and chemical molecules. Suitable protein inhibitors may be, for example, monoclonal or polyclonal antibodies which bind to one of the enzymes described below. Inhibition of enzymes can be achieved by any of a variety of mechanisms known in the art, including, but not limited to, binding directly to the enzyme (e.g., enzyme inhibitor compound binding complex or substrate mimetic), denaturing or otherwise inactivating the enzyme, inhibiting the expression of a gene which encodes the enzyme (e.g., transcription to m RNA, translation to a nascent polypeptide) and/or final modifications to a mature protein.

As used herein, the term “inhibit” or “inhibiting” means any effect in down-regulating, decreasing, reducing, suppressing, or inactivating (also partially) the amount and/or activity of the Carnitine-Palmitoyl-Transferase-1 enzyme.

As used herein, the term “regulating the expression and/or activity” generally refers to any process that functions to control or modulate the quantity or activity (functionality) of a cellular component, particularly an enzyme. Static regulation maintains expression and/or activity at some given level. Up-regulation refers to a relative increase in expression and/or activity. Accordingly, down-regulation refers to a decrease in expression and/or activity. Down-regulation is synonymous with the inhibition of a given cellular component's expression and/or activity.

In general, CPT-I inhibitors can be identified by screening test compounds, for example a compound of formula (I) or a library of test compounds, for their ability to inhibit the Carnitine-Palmitoyl-Transferase-1 activity. In this context, cells or cell lysates may be tested for their ability to degrade palmitate by incubating the cells or cell lysates with radioactive palmitate and measuring the production of radioactive ketone bodies and/or the release of ¹⁴CO₂. Furthermore, it is possible to perform an in silico screen, based on the structure of a known enzyme involved in fatty acid oxidation.

### Carnitine-Palmitoyl-Transferase-1 (CPT-1) Inhibitors for Use in a Method of Preventing or Treating Sepsis

As indicated above, there is a need for alternative and/or complimentary compounds and methods for the effective prevention or treatment of sepsis and symptoms thereof. In particular, there is a need for compositions and methods for combatting bacterial infections in, for example, wound patients.

Therefore, a problem of the present invention was to provide compounds having the above-mentioned desired characteristics that are suitable for use in the effective prevention or treatment of sepsis.

The present invention, in one aspect, relates to a Carnitine-Palmitoyl-Transferase-1 (CPT-1) inhibitor for use in a method of preventing or treating sepsis in a mammalian subject.

Further included are pharmaceutically or veterinarily acceptable salts, hydrates or solvates of Carnitine-Palmitoyl-Transferase-1 (CPT-1) inhibitors for said use, in particular of the compounds of formula (I) or its intermediate compounds disclosed herein.

As shown in the Examples, the inventors have now surprisingly and unexpectedly found that Carnitine-Palmitoyl-Transferase-1 (CPT-1) inhibitors, in particular the compounds of formula (I) or a pharmaceutically or veterinarily acceptable salt, hydrate or solvate thereof, are useful prevention or treatment of sepsis in a mammalian subject.

According to the present invention, sepsis includes any recognized form of sepsis, e.g., hospital-acquired sepsis, medical procedure related sepsis, medical device related sepsis, severe sepsis, or septic shock. Sepsis also includes any recognized condition or symptom associated with sepsis. In general, symptoms of sepsis include but are not limited to fever above 101.3° F. (38.5° C.) or below 95° F. (35° C.), heart rate higher than 90 beats per minute, respiratory rate higher than 20 breaths a minute, and probable or confirmed infection (i.e., presence of one or more infectious agents such as bacteria, fungi or viruses). Typically, a clinical diagnosis of sepsis includes the presence of a least two symptoms selected from the sepsis symptoms. Symptoms of severe sepsis include but are not limited to significantly decreased urine output, abrupt change in mental status, decrease in platelet count, difficulty breathing, abnormal heart pumping function and abdominal pain. Typically, a clinical diagnosis of severe sepsis includes the presence of a least one additional symptom selected from the severe sepsis symptoms, the presence of which is indicative of organ failure. Symptoms of septic shock can include but are not limited to extremely low blood pressure that does not respond to simple fluid replacement. Typically, a clinical diagnosis of septic shock includes the presence of at least one additional symptom selected from the septic shock symptoms.

In general, sepsis can be caused by a variety of infectious agents, including bacteria, fungi, viruses and parasites, and can proceed from merely infection to multiple organ dysfunction syndrome (MODS) and eventual death if untreated. In some embodiments, sepsis may involve, for example, bacteremia or fungal infection, such as candidemia or aspergillis infection. In some embodiments, sepsis may result from severe injury, severe wound, or burn, and may be a post-surgical infection.

According to the present invention, treatment of sepsis includes any form of treating or preventing sepsis, e.g., reducing any symptom of sepsis, reducing the severity of any symptom of sepsis, delaying the onset of sepsis, shortening the duration of one or more symptoms of sepsis, reducing the opportunity or occurrence of sepsis, treating or inhibiting any cause or condition associated with sepsis, reducing any clinical criteria or measurement of the degree or condition of sepsis, e.g., ICU frequency, ICU stay, ICU free days, duration of ventilation, ventilation free days, mortality, e.g., 28 day mortality, in-ICU mortality, in-hospital mortality, etc., dynamic change of SOFA, HLA-DR expression, etc.

In one embodiment, the invention involves administering adosage of a Carnitine-Palmitoyl-Transferase-1 (CPT-1) inhibitor to a subject to enhance immune responses to pathogen exposure, or potential pathogen exposure in order to prevent or to treat sepsis. The subject is at risk for the sepsis, severe sepsis or septic shock. The subject is preferably a mammalian subject, particularly preferred a human.

The Carnitine-Palmitoyl-Transferase-1 (CPT-1) inhibitor can be administered prior to, along with and/or after an event predicted to result in pathogen exposure or introduction of an opportunistic environment. In a preferred embodiment, the CPT-1 inhibitor is first administered prior to the event, and again on the day of the event, and optionally after the event.

In a particular preferred embodiment, the event is selected from admittance to a hospital or health care facility, surgery, placement of an invasive medical device, kidney dialysis, and initiation of chemotherapy or radiation therapy for cancer treatment.

In the present invention, the treatment regimen includes dosage per administration per day, as well as a number of days per treatment cycle or combinations thereof.

In general, the CPT-1 inhibitor can be administered at a dosage of from about 0.2 mg to 20 mg, 0.2 mg to 15 mg, 0.4 to 10 mg, 0.5 mg to 8 mg. 0.5 mg to 6 mg, 0.5 mg to 3 mg. In some embodiments, the CPT-1 inhibitor is administered at 0.2 mg, 0.5 mg, 0.4 mg, 0.8 mg, 1 mg, 1.6 mg, 3 mg, 3.2 mg, 6.4 mg or about 8 mg. In some embodiments, the CPT-1 inhibitor is administered to a human patient at a dose corresponding to at least about 0.5 mg (e.g., at least about 0.8 mg, or at least about 1.6 mg), at least about 3 mg (e.g., at least about 3.2 mg), or at least about 5 mg (e.g., at least about 6.4 mg) of the CPT-1 inhibitor. In some embodiments, the CPT-1 inhibitor is administered within the range corresponding to about 0.1 to 20 mg of the CPT-1 inhibitor, or about 1 to 10 mg of the CPT-1 inhibitor, or about 2 to 10 mg of the CPT-1 inhibitor, or about 2 to 8 mg of the CPT-1 inhibitor, or about 2 to 7 mg of the CPT-1 inhibitor. In certain embodiments, the dosage unit is within a range of about 3 to 6.5 mg, such as about 3.2 or 6.4 mg of the CPT-1 inhibitor. In certain embodiments, the CPT-1 inhibitor dose is adjusted to the size of the patient, and may be provided at from 10 to 100 μg/kg (e.g., about 20, 40, 60, or 80 μg/kg). Dosages may also be adjusted for the condition of each patient as well as other drugs taken by the patient. In addition, dosages may be adjusted according to the species of the subject, but in each case, approximately correspond to the human equivalent of CPT-1 inhibitor (mg/kg).

In some embodiments, such dosage is administered hourly, daily, weekly or monthly.

In some embodiments, the CPT-1 inhibitor is administered hourly, about every 1 to 24 hours, 1 to 20 hours, 1 to 16 hours, 1 to 12 hours, 1 to 8 hours, 1 to 6 hours, 1 to 4 hours, 1 to 2 hours or every hour. In some embodiments, the CPT-1 inhibitor is administered about every 2, 3, 5, 5, or 6 hours, or is administered about every 10 minutes, 15 minutes, 30 minutes, 45 minutes or 60 minutes.

In another embodiment, the CPT-1 inhibitor can be administered by the oral, sublingual, transdermal or parenteral route, preferably by the intramuscular, intraperitoneal or intravascular route; more preferably by the intravenous route.

Alternatively, the CPT-1 inhibitor can be administered by a plurality of injections (sub-doses of CPT-1 inhibitor) on a treatment day, so as to substantially continuously maintain an immune stimulating-effective amount of CPT-1 inhibitor in the patient's circulatory system for a longer period of time. Suitable injection regimens may include an injection every 2, 3, 4, 6, etc. hours on the day of administration (e.g., from 2 to 5 injections), so as to substantially continuously maintain the immune stimulating-effective amount of the CPT-1 inhibitor in the patient's circulatory system on the day of CPT-1 inhibitor treatment.

In some embodiments, the CPT-1 inhibitor may be administered by continuous infusion. Briefly, continuous infusion of CPT-1 inhibitor maintains an immune stimulating-effective amount of a CPT-1 inhibitor in a patient's circulatory system for a longer period. In some embodiments, the CPT-1 inhibitor may be administered to the patient for treatment periods of at least about 2, 4, 6, 10, 12 hours, or longer, which may improve effectiveness in some embodiments. The infusion may be carried out by any suitable means, such as by minipump.

In some embodiments, the CPT-1 inhibitor is administered by continuous infusion for about 1 to 168 hours, 1 to 144 hours, 1 to 120 hours, 1 to 96 hours, 1 to 72 hours, 1 to 48 hours, 1 to 24 hours, 1 to 20 hours, 1 to 16 hours, 1 to 12 hours 1 to 10 hours, 1 to 8 hours, 1 to 6 hours, 1 to 4 hours to 1 to 2 hours. In some embodiments, the CPT-1 inhibitor is administered by continues infusions for about 10 minutes, 15 minutes, 30 minutes, 45 minutes or 60 minutes. In some embodiments, the CPT-1 inhibitor is administered by continuous infusion for about 1 hour, 2 hours, 3 hours, 4 hours, 5 hours, 6 hours, 7 hours, 8 hours, 10 hours, 12 hours, 24 hours or more. In some embodiments, the continuous infusion periods are separated by periods of non-infusion (i.e., periods where no CPT-1 inhibitor is administered). In some embodiments, the non-infusion period ranges from 1 to 168 hours, 1 to 144 hours, 1 to 120 hours, 1 to 96 hours, 1 to 72 hours, 1 to 48 hours, 1 to 24 hours, 1 to 20 hours, 1 to 16 hours, 1 to 12 hours 1 to 10 hours, 1 to 8 hours, 1 to 6 hours, 1 to 5 hours, 1 to 4 hours, 1 to 3 hours, 1 to 2 hours. In some embodiments, the non-infusion period is about 1 hour, 2 hours, 3 hours, 4 hours, 5 hours, 6 hours, 7 hours, 8 hours, 10 hours, 12 hours, 24 hours or more.

In some embodiments, a predetermined amount of CPT-1 inhibitor, e.g., immune stimulating-effective amount of a CPT-1 inhibitor may be substantially continuously maintained in a patient's circulatory system by administering the CPT-1 inhibitor to the patient at a rate within a range of about 0.0001-0.1 mg/hr/Kg patient body weight. Exemplary administration rates are within a range of about 0.0003-0.03 mg/hr/Kg patient body weight. For continuous infusion, the CPT-1 inhibitor is present in a pharmaceutically acceptable liquid carrier, such as water for injection, or saline in physiological concentrations.

In some embodiments, the CPT-1 inhibitor is administered about every 1 to 20 days, every 1 to 15 days, every 1 to 10 days, every 1 to 7 days, every 1 to 5 days, every 1 to 3 days or daily. In some embodiments, the CPT-1 inhibitor is administered for about 1 to 100 days, 1 to 90 days, 1 to 80 days, 1 to 70 days, 1 to 50 days, 1 to 40 days, 1 to 30 days, 1 to 20 days, 1 to 15 days, 1 to 10 days, 1 to 7 days, 1 to 5 days, 1 to 3 days, 1 to 14 days, 5 to 14 days or 1 to 2 days. In some embodiments, the CPT-1 inhibitor is administered for about 1, 2, 3, 4, 5, 6, 7, 8, 9, 10, 11, 12, 13, 14, 15, 16, 17, 18, 19, 20, 25, 30 or more days. In some embodiments, CPT-1 inhibitor is administered about twice per day for at least 5 days (e.g., from 5 to 14 days). In some embodiments, CPT-1 inhibitor is administered about twice per day for about 5 to 10 days (or about 5 days) followed by about once per day for at least two days, or about 2 to 7 days, or about 2 days.

In some embodiments, the CPT-1 inhibitor is administered for about 1 to 8 weeks, about 1 to 6 weeks, about 1 to 5 weeks, about 1 to 4 weeks, about 2 to 4 weeks, or about 1−2 weeks. In some embodiments, the CPT-1 inhibitor is administered for 1 week, 2 weeks, 3 weeks, 4 weeks, 5, weeks, 6 weeks, 7 week, 8 weeks or more. In some embodiments, the CPT-1 inhibitor is administered for about 1 month, 2 months, 3 months or 4 months or more. In some embodiments, the CPT-1 inhibitor is administered for about 1 to 4 months, 1 to 3 months, 1 to 2 months, or about one month.

In some embodiments, the OPT-1 inhibitor is administered about 1 to 8 times per day for about 1 to 8 weeks. In some embodiments, the CPT-1 inhibitor is administered about 1 to 7 times per day, 1 to 6 times per day, 1 to 5 times per day, 1 to 4 times per day, 1 to 3 times per day, 1 to 2 times per day, or about 1 times per day for about 1 to 7 weeks, 1 to 6 weeks, 1 to 5 weeks, 1 to 4 weeks, 1 to 3 weeks, 1 to 2 weeks, or about 1 weeks. In some embodiments, the CPT-1 inhibitor is administered about 1 to 8 times per day, 1 to 7 times per day, 1 to 6 times per day, 1 to 5 times per day, 1 to 4 times per day, 1 to 3 times per day, 1 to 2 times per day, or about 1 times per day for about 1 to 30 days, 1 to 25 days, 1 to 20 days, 1 to 15 days, 1 to 7 days or 1 to 5 days. In some embodiments, the CPT-1 inhibitor is administered for 1 to 4 times per day for 1 to 30 days. In some embodiments, the CPT-1 inhibitor is administered for about 1-2 times per day for 1 to 15 days or 1 to 7 days or 1 to 5 days. In some embodiments, the CPT-1 inhibitor is administered about 1 to 2 times per day for 5 days followed by once per day for 2 days. In some embodiments, the CPT-1 inhibitor is administered about twice daily for 5 days followed by once per day for 2 days. In some embodiments, the CPT-1 inhibitor is administered about four times per day for 5 days or 7 days.

In some embodiments, the regimen employs a dosage of CPT-1 inhibitor that is at least 0.2 mg, 0.5 mg, 0.8 mg, 1.6 mg, 3.2 mg, or 6.4 mg, with 1, 2, 3, 4, 5, 6, 7 or 8 or more. In some embodiments, 3 doses or less can be administered. In some embodiments more dosages may be administered, such as 5, 6, 7, 8, 9 or 10, 11, 12, 13, 14, 15, 20, 25, 30, 35, 40, 45, 50 or more. In some embodiments, the dose of CPT-1 inhibitor is a relatively low dose of at least 0.2 mg, 0.4 mg, 0.5 mg, 0.8 mg, or 1.6 mg. The CPT-1 inhibitor administrations may be spaced apart by about 1, 2, 3, 4, 5, 6, 7, 8, 9, 10, 12, 16, 20 or 24 hours or about 1, 2, 3, 4, 5, 6, 7, 8, 9 or 10 days, and may be given weekly in some embodiments, as is described in greater detail herein. In some embodiments, the CPT-1 inhibitor is administered at a dose within the range of about 0.5 mg to 3 mg. In some embodiments, the CPT-1 inhibitor is administered at a dose within the range of about 1 mg to 2 mg.

In some embodiments the CPT-1 inhibitor is administered at a dosage of about 0.5 mg, about 0.8 mg, about 1.6 mg, about 3 mg, about 3.2 mg, about 5 mg, or about 6.4 mg or more of CPT-1 inhibitor and optionally in combination with one or more treatment schedules described in this paragraph. In some embodiments, the CPT-1 inhibitor is administered 1, 2, 3, 4 or more times per day for 1, 2, 3, 4, 5, 6, 7, 8, 9, or 10, 11, 12, 13, 14, 15, 16, 17, 18, 19, 20, 25 or 30 days or more. In some embodiments, the CPT-1 inhibitor is administered for 1, 2, 3, 4, 5, 6, 7 or 8 weeks or more. In some embodiments, the CPT1-inhibitor is administered for 1 or 2 months or more. In some embodiments, the CPT-1 inhibitor is administered 1, 2, 3, 4 or more times per day for 2, 3, 4, 5, 6, 7 or 8 days. In some embodiments, the CPT-1 inhibitor is administered 1, 2, 3, 4 or more times per day for 4, 5, 6 or 7 days. In some embodiments, the CPT-1 inhibitor is administered 1, 2, 3, 4 or more times per day for 5, 6, or 7 days. In some embodiments, the CPT-1 inhibitor is administered 1, 2, 3, 4 or more times per day for 1, 2, 3, 4, 5, 6, 7, 8, 9, or 10 days or more, followed by 1, 2, 3, 4 or more times per day for 1, 2, 3, or 4 days or more. In some embodiments, the CPT-1 inhibitor is administered 1, 2, 3, 4 or more times per day for 2, 3, 4, 5, 6, 7, or 8 days, followed by 1, 2, 3, 4 or more times per day for 1, 2, 3, or 4 days. In some embodiments, the CPT-1 inhibitor is administered 1, 2, 3, 4 or more times per day for 4, 5, 6 or 7 days, followed by 1, 2, 3, 4 or more times per day for 1, 2, 3, or 4 days. In some embodiments, the CPT-1 inhibitor is administered 1, 2 or 3 times per day for 4, 5, 6 or 7 days, followed by 1, 2 or 3 times per day for 1, 2, 3, or 4 days. In some embodiments, the CPT-1 inhibitor is administered 2 times per day for 7 days. In some embodiments, the CPT-1 inhibitor is administered 2 times per day for 5 days. In some embodiments, the CPT-1 inhibitor is administered 1 time per day for 5 days. In some embodiments, the CPT-1 inhibitor is administered 2 times per day for 5 days, followed by once per day for 2 days. In some embodiments, about 1.6 mg of the CPT-1 inhibitor is administered 2 times per day for 5 days, followed by once per day for 2 days.

The timing of CPT-1 inhibitor administration may be selected to enhance the immune response including antibody titers (e.g., the development or level of antibody titers) to cover a period of increased risk of sepsis. For example, in certain embodiments, the CPT-1 inhibitor administrations are given about 5 days to about 9 days apart, and in various embodiments are administered about 1, 2, 3, 4, 5 6, 7, or 8 days apart. The administrations may be given about 7 days apart (e.g., approximately weekly administration). In other embodiments, the CPT-1 inhibitor administrations are given 1, 2, 3, or 4 days apart

In other embodiments, the regimen can be initiated at about 1 to 10 days (in some embodiments 5 to 9 days) prior to an event predicted (or with a significant risk) to result in sepsis, in order to provide for treatment/prevention of sepsis. Exemplary events are described herein. In some of these embodiments, the efficient regimen involves from about 1 to 5 administrations of CPT-1 inhibitor, such as 3 or less. The CPT-1 inhibitor administrations may be spaced apart by about 1, 2, 3, 4, 5, 6, 7, 8, 9 or 10 days, and may be given weekly in some embodiments.

In some embodiments, the CPT-1 inhibitor is first administered prior to an event (as described), such as admittance to a healthcare facility, scheduled surgery, or placement of invasive medical device, and again on the day of the event, and optionally after the event. For example, CPT-1 inhibitor may be administered from 1 to 10 days prior to the event, such as from about 5 to about 9 days prior to the event, and again on the day of the event. The CPT-1 inhibitor may be administered about 7 days prior to the event, and again on the day of the event, and optionally within 2 to 10 days after the event (e.g., from 4 to 8 days after the event). For example, patients receiving two doses of the CPT-1 inhibitor in accordance with certain embodiments of the invention are likely to achieve a faster and/or larger response to sepsis, and which may be protective for at least 21 days, at least 42 days, or longer.

In some embodiments, the CPT-1 inhibitor is administered prior to, along with and/or after an event predicted to result in pathogen exposure or introduction of an opportunistic environment, as described herein. For example, the event may be admittance to a hospital or health care facility for a period of time (e.g., at least 3 days, at least one week, or at least ten days, or at least one month). In other embodiments, the event is a scheduled surgery or invasive medical procedure, as described. In other embodiments, the event is the placement of an invasive medical device as described. In still other embodiments, the event is kidney dialysis or initiation of chemotherapy or radiation therapy for cancer treatment (as described).

In another embodiment, the invention relates to a Carnitine-Palmitoyl-Transferase-1 (CPT-1) inhibitor for use as defined herein, wherein in the method the Carnitine-Palmitoyl-Transferase-1 (CPT-1) inhibitor is administered within at least the first 24 hours, 48 hours, 72 hours, or 96 hours of showing one or more signs or symptoms of the sepsis, severe sepsis or septic shock.

In some embodiments, the CPT-1 inhibitor is administered within the first about 1 hour, 2 hours, 4, hours, 6 hours, 8 hours, 10 hours, 12 hours, 24 hours, 72 hours, 96 hours, 120 hours, 144 hours, or 168 hours, of a determination of sepsis. In some embodiments, the CPT-1 inhibitor is administered within the first about 10 minutes, 15 minutes, 30 minutes, 45 minutes or 60 minutes of a determination of sepsis.

In still other embodiments, the regimen involves from 1 to 4 administrations of CPT-1 inhibitor, such as 3 or less, and the regimen is timed to begin prior to an event anticipated to lead to sepsis. For example, the regimen may be initiated from 2 to 10 days prior to the event, such as from 5 to 10 days prior, and a second dose may be administered on the day of the event. The CPT-1 inhibitor administrations may be spaced apart by about 1, 2, 3, 4, 5, 6, 7, 8, 9 or 10 days, and may be given weekly in some embodiments. In still other embodiments, the regimen involves a dose of CPT-1 inhibitor, provided approximately weekly (e.g., every 5 to 9 days), for 2, 3, 4 or more weeks.

In still other embodiments, the patient receives 2 doses of a CPT-1 inhibitor (such as 2 mg to 8 mg per dose), and such doses are spaced by about 1, 2, 3, 4, 5, 6, 7, 8, 9, 10, 12, 16, 20 or 24 hours or about 1, 2, 3, 4, 5, 6, 7, 8, 9 or 10 days, or approximately weekly. This regimen may be repeated approximately monthly, or every other month, and may be particularly beneficial for protecting chronically ill and immunodeficient patients from sepsis. In some embodiments, the CPT-1 inhibitor is administered at a dose within the range of about 0.5 mg to 3 mg. In some embodiments, the CPT-1 inhibitor is administered at a dose within the range of about 1 mg to 2 mg.

In certain aspects of the invention, the CPT-1 inhibitor regimen is part of an institutional program to reduce the rate or incidence of sepsis, e.g., hospital-acquired sepsis.

In some embodiments, the regimen of CPT-1 inhibitor involves administering the agent to the subject at a dose sufficient to enhance antibody titers, and/or sufficient to speed the development of antibody titers, to pathogen exposure. In some other embodiments, the regimen of involves a regimen provides serum level of CPT-1 inhibitor at about 0.01 to 10.0 ng/ml, 0.1 to 1.0 ng/ml, or 0.05 to 5 ng/ml during treatment. In some embodiments, the peak plasma levels of CPT-1 inhibitor is at least about 10 ng/ml, 20 ng/ml, 30 ng/ml, 40 ng/ml, 50 ng/ml, 60 ng/ml, 70 ng/ml, 80 ng/ml, 90 ng/ml, or 100 ng/ml.

The CPT-1 inhibitor may be provided in lyophilized form, and reconstituted with sterile (e.g., aqueous) diluent prior to administration. The CPT-1 inhibitor may be administered by any effective route, including by subcutaneous injection, intramuscular injection, intravenous injection or infusion, and orally. Preferably, the CPT-1 inhibitor is administered by oral dosing.

In some embodiments, the CPT-1 inhibitor is administered at a dose twice a day for a period of time and then administered at the same dose once a day for a period of time.

In certain embodiments, the patient receives the CPT-1 inhibitor at a dose of from 2 to 8 mg (e.g., at 0.8, 1.6, 3.2 or 6.4 mg per dose) either once or two times daily, or every other day, for from 3 to 14 days (e.g., 3, 5, 7, 10, or 14 days). Such regimen may be timed with respect to an event that places the patient at further risk for exacerbation of the infection or complicating illness, such as those events described herein (e.g., surgery, hemodialysis, initiation of cancer treatment, placement of medical device). For example, the event may be scheduled at a time between day 2 and day 10 of the regimen, including day 3, day 5, day 7, or day 10. The regimen may be concurrent with antibacterial, antiviral, or antifungal therapy, including with active agents described herein. In some embodiments, the CPT-1 inhibitor is administered within the first 24 hours, 48 hours, 72 hours, 96 hours, 120 hours or 144 hours.

In accordance with the invention, the CPT-1 inhibitor of the present invention is administered to a subject with a regimen sufficient to treat sepsis. The CPT-1 inhibitor regimen in some embodiments is an “efficient” regimen. That is, the regimen achieves its goal with relatively few administrations of CPT-1 inhibitor and/or by timing the administration of CPT-1 inhibitor with events anticipated to result in sepsis. The “event” is not a vaccination, but an exposure or increased susceptibility to the potential infectious agent that has the potential to lead or does lead to sepsis, severe sepsis or septic shock. The efficient regimen of CPT-1 inhibitor is relatively convenient and comfortable for the patient, as well as more affordable and effective.

According to the present invention, the CPT-1 inhibitor used in methods of the present invention can be administered either alone or in combination with a standard of care for sepsis, or as part of treatment regimen involving the standard of care for sepsis. In some embodiments, the standard of care is a protease inhibitor, activated protein C, corticosteroids, intensive insulin therapy synthetic fluid replacement substance (pentastarch), drotrecognin alfa (activated; DrotAA), volume resuscitation, hydrocortisone and fludrocortisone. (See, e.g., Hotchkiss, R. S. and Karl, I. E., The Pathophysiology and treatment of Sepsis, NEJM, 348:2 (2008)).

In another embodiment, the invention relates to a Carnitine-Palmitoyl-Transferase-1 (CPT-1) inhibitor for use as defined herein, wherein the Carnitine-Palmitoyl-Transferase-1 (CPT-1) inhibitor is administered as a single agent.

In another embodiment, the invention relates to a Carnitine-Palmitoyl-Transferase-1 (CPT-1) inhibitor for use as defined herein, wherein the Carnitine-Palmitoyl-Transferase-1 (CPT-1) inhibitor is administered in combination with another drug, preferably in combination with an antimicrobial or an antiviral drug.

Antimicrobial or antiviral drugs include but are not limited to all antimicrobial or an antiviral drug known to the skilled person. Preferred antimicrobial drugs may be selected from the group consisting of polymyxins, beta-lactams, aminoglycosides, glycopeptides, and polyenes. In a more preferred embodiment, said nephrotoxic antimicrobial agent is not a beta-lactam antibiotic. In a preferred embodiment the anti.microbial drug may be selected from penicillin G, penicillin, amikacin amphotericin B, ampicillin, amoxicillin, atavaquone, bacampicillin, bacitracin, cyclacillin, epicillin, hetacillin, pivampicillin, methicillin, nafcillin, oxacillin, cloxacillin, dicloxacillin, flucloxacillin, carbenicillin, gentamicin, ticarcillin, avlocillin, mezlocillin, piperacillin, amdinocillin, cephalexin, cephradine, cefadoxil, cefaclor, cefazolin, cefuroxime axetil, cefamandole, cefonicid, cefoxitin, cefotaxime, ceftizoxime, cefmenoxine, ceftriaxone, cidofovir, moxalactam, cefotetan, cefoperazone, ceftazidme, imipenem, clavulanate, timentin, sulbactam, neomycin, erythromycin, metronidazole, netilmicin chloramphenicol, clindamycin, tobramycin kanamycin, lincomycin, lamivudine, vancomycin, trimethoprim-sulfamethoxazole, aminoglycosides, quinolones, tetracyclines, methenamine, polymyxin B, polymyxin E (colistin), pentamidine, pentamidine isoethionate rifampin, sulfisoxazole, sulfamethoxazole, spectinomycin, vidarabine, AZT, and 3TC, and salts or derivatives thereof.

Preferred antiviral drugs may be selected from the group consisting of Abacavir, Aciclovir, Acemannan, Acyclovir, Adefovir, Amantadine, Aprenavir, Alvircept Ampligen, Arbidol, Aranotin, Arildone, Atevirdine Mesylate, Atazanavir, Atripla, Avridine, Balavir, Boceprevirertet, Cidofovir, Cipamfylline, Combivir, Cytarabin, Dolutegravir, Darunavir, Dleavirdine, Desciclovir, Didanosine, Disoxaril, Docosanol, Edoxudine, Edozudine, Efavirenz, Emtricitabine, Enviradene, Enviroxime, Efuvirtide, Entecavir, Ecoliver, Famciclovir, Famotine, Fiacitabine, Fialuridine, Fosarilate, Fomivirsen, Fosamprenavir, Foscarnet, Fosfonet, Fusion Inhibitors, Ganciclovir, Ibacitabine, Imunovir, Idoxuridine, Imiquimod, Indinavir, Inosine, Integrase inhibitor, Interferon type Ill, Interferon type II, Interferon type I, Interferon, Kethoxal, Lamivudine, Lopinavir, Loviride, Lobucavir, Maraviroc, Moroxydine, Methisazone, Memotine, Nelfinavir, Nevirapine, Nexavir Nucleoside analogues, Oseltamivir (Tamiflu), Peginterferon alfa-2a, Penciclovir, Penciclovir, Peramivir, Pirodavir, Podophyllotoxin, Protease inhibitors, Raltegravir, Reverse transcriptase inhibitor, Ribavirin, Rimantadine, Ritonavir, Pyramidine, Saquinavir, Sofosbuvir, Somantadine, Sorivudine, Statolon, Sudotox, Stavudine, Synergistic enhancer (antiretroviral), Telaprevir, Tenofovir, Tenofovir disoproxil, Tilorone, Tipranavir, Trifluridine, Trizivir, Tromantadine, Truvada, traporved, Valaciclovir (Valtrex), Valganciclovir, Vicriviroc, Vidarabine, Viramidine, Viroxime, Zalcitabine, Zanamivir (Relenza), and Zidovudine, Zinviroxime, and salts or derivatives thereof.

In one embodiment, the invention relates to a Carnitine-Palmitoyl-Transferase-1 (CPT-1) inhibitor for use as defined herein, wherein the Carnitine-Palmitoyl-Transferase-1 (CPT-1) inhibitor is selected from the group consisting of etomoxir, C75 (4-methylene-2-octyl-5-oxotetrahydrofuran-3-carboxylic acid), ST1326 ((R)-3-(3-tetradecylureido)-4-(trimethylammonio)-butanoate) and perhexiline.

In one embodiment, the invention relates to a Carnitine-Palmitoyl-Transferase-1 (CPT-1) inhibitor for use as defined herein, wherein the Carnitine-Palmitoyl-Transferase-1 (CPT-1) inhibitor is a compound of the formula (I) as defined in European patent application no. 21172027.1.

In the following, preferred groups of the compounds of formula (I) are described. The preferred groups constitute preferred embodiments of the compounds of formula (I). Any combinations of the embodiments of the compounds of formula (I) used in the invention described herein are considered to be within the scope of the invention.

In one embodiment, the invention relates to a Carnitine-Palmitoyl-Transferase-1 (CPT-1) inhibitor for use as defined herein, wherein the CPT-1 inhibitor is a compound of the formula (I) as defined herein, wherein
R¹ is unsubstituted or substituted aryl, preferably unsubstituted or substituted phenyl, naphthyl, tetrahydronaphthyl, indenyl, indanyl, pentalenyl, or fluorenyl,
   unsubstituted or substituted heteroaryl, preferably unsubstituted or substituted pyridyl, pyrazinyl, pyrimidinyl, pyridazinyl, 2-oxo-1,2-dihydropyridinyl, oxazolyl, oxydiazolyl, isoxazolyl, thiadiazolyl, tetrazolyl, pyrazolyl, imidazolyl, thiazolyl and thienyl, quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, or indazolyl,
   unsubstituted or substituted 5- or 6-membered saturated or partially unsaturated heterocyclyl, or
   unsubstituted or substituted C₃-C₈-cycloalkyl, or cyclohexenyl;
L is a single bond, or a difunctional linker, preferably *—O—, *—OCH₂—, *—CH₂O—, *—CH₂—, *—CH₂—CH₂—, *—CH₂—CH₂—CH₂—, or *—CH₂—C(CH₃)₂—, or a trifunctional linker, preferably, *—CH═, wherein the * indicates the point of attachment to the carbonyl (C═O) group;
R² is unsubstituted or substituted phenyl, naphthyl, or pyridyl, or C₁-C₄-alkyl;
R³ is H, C₁-C₈-alkyl, halogen-C₁-C₄-alkyl, or C₃-C₈-cycloalkyl,
   unsubstituted or substituted 4, 5- or 6-membered saturated or partially unsaturated heterocyclyl, or
   unsubstituted or substituted phenyl;
Y is —(C═O)—, —(SO₂)— or a single bond;
or a stereoisomer, a pharmaceutically or veterinarily acceptable salt, hydrate or solvate thereof.

In one embodiment, the invention relates to a Carnitine-Palmitoyl-Transferase-1 (CPT-1) inhibitor for use as defined herein, wherein the CPT-1 inhibitor is a compound of the formula (I) as defined herein, wherein
R¹ is unsubstituted or substituted aryl, preferably unsubstituted or substituted phenyl, naphthyl, tetrahydronaphthyl, indenyl, indanyl, pentalenyl, or fluorenyl,
   unsubstituted or substituted heteroaryl, preferably unsubstituted or substituted pyridyl, pyrazinyl, pyrimidinyl, pyridazinyl, 2-oxo-1,2-dihydropyridinyl, oxazolyl, oxydiazolyl, isoxazolyl, thiadiazolyl, tetrazolyl, pyrazolyl, imidazolyl, thiazolyl and thienyl, quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, or indazolyl,
   unsubstituted or substituted 5- or 6-membered saturated or partially unsaturated heterocyclyl, or
   unsubstituted or substituted C₃-C₈-cycloalkyl, or cyclohexenyl;
   each R¹ being optionally and independently substituted with one to three of the following residues:
   —CN,
   halogen, preferably —F or —Cl,
   C₁-C₄-alkyl, preferably methyl,
   halogen-C₁-C₄-alkyl, preferably difluoromethyl, or trifluoromethyl,
   SO₂Me, or
   CO₂C₁-C₄-alkyl, preferably CO₂Me;
L is a single bond, or a difunctional linker, preferably *—O—, *—OCH₂—, *—CH₂O—, *—CH₂—, *—CH₂—CH₂—, *—CH₂—CH₂—CH₂—, or *—CH₂—C(CH₃)₂—, wherein the * indicates the point of attachment to the carbonyl (C═O) group;
R² is unsubstituted or substituted phenyl, naphthyl, or pyridyl;
   each R² being optionally and independently substituted with one to three, preferably with one of the following residues:
   —CN,
   halogen, preferably —F or —Cl,
   C₁-C₄-alkyl, preferably methyl,
   halogen-C₁-C₄-alkyl, preferably difluoromethyl, or trifluoromethyl,
   SO₂Me,
   CO₂C₁-C₄-alkyl, preferably CO₂Me,
   adamantyl,
   unsubstituted or substituted phenyl, being optionally substituted with one or
   more, preferably with one substituent selected from
   halogen, preferably —F or —Cl,
   halogen-C₁-C₄-alkyl, preferably trifluoromethyl,
   C₃-C₈-cycloalkyl, preferably cyclohexyl, or
   Pyridyl;
R³ is H, C₁-C₈-alkyl, halogen-C₁-C₄-alkyl, or C₃-C₈-cycloalkyl,
   unsubstituted or substituted 4, 5- or 6-membered saturated or partially unsaturated heterocyclyl, or
   unsubstituted or substituted phenyl;
   each 5- or 6-membered saturated or partially unsaturated heterocyclyl or phenyl being optionally and independently substituted with one or more, preferably with one of the following residues:
   —CN,
   halogen, preferably —F or —Cl;
   C₁-C₄-alkyl, preferably methyl;
   halogen-C₁-C₄-alkyl, preferably chloromethyl, dichloromethyl, trichloromethyl, fluoromethyl, difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2fluoroethyl, 2-chloro-2,2-difluoroethyl, 2,2-dichloro-2-fluoroethyl, 2,2,2-trichloroethyl, pentafluoroethyl, or 2,2-difluor-3-methyl-butyl, particularly preferred difluoromethyl, or trifluoromethyl,
   SO₂Me,
   CO₂C₁-C₄-alkyl, preferably CO₂Me, or
   CO—C₁-C₄-alky, preferably CO-Me,
and
Y is —(C═O)—, —(SO₂)— or a single bond;

In one embodiment, the invention relates to a Carnitine-Palmitoyl-Transferase-1 (CPT-1) inhibitor for use as defined herein, wherein the CPT-1 inhibitor is a compound of the formula (I) as defined herein, wherein
R¹ is unsubstituted or substituted phenyl, naphthyl, or tetrahydronaphthyl,
   unsubstituted or substituted pyridyl, pyrazinyl, pyrimidinyl, oxazolyl, oxydiazolyl, isoxazolyl, thiadiazolyl, tetrazolyl, pyrazolyl, imidazolyl, thiazolyl, thienyl, or quinolinyl,
   unsubstituted or substituted pyrrolidinyl, piperidinyl, tetrahydropiperidinyl, or piperazinyl, or
   unsubstituted or substituted cyclopentyl; cyclohexyl or cyclohexenyl;
L is a single bond, or a difunctional linker, preferably *—O—, *—OCH₂—, *—CH₂O—, *—CH₂—, or *—CH₂—CH₂—, wherein the * indicates the point of attachment to the carbonyl (C═O) group;
R² is unsubstituted or substituted phenyl, naphthyl, or pyridyl;
R³ is H, C₁-C₄-alkyl, halogen-C₁-C₄-alkyl, cyclopropyl, cyclobutyl, cyclopentyl or cyclohexyl,
   unsubstituted or substituted azetidinyl, pyrrolidinyl, piperidinyl, or oxetanyl,

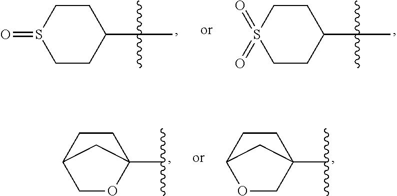 [SMILES: *C1CCS(=O)CC1 ; *C1CCS(=O)(=O)CC1 ; *C12CCC(CO1)C2 ; *C12CCC(C1)OC2]

or
unsubstituted or substituted phenyl; and
Y is —(C═O)—, —(SO₂)— or a single bond, preferably —(C═O)—.

In another embodiment, the invention relates to a Carnitine-Palmitoyl-Transferase-1 (CPT-1) inhibitor for use as defined herein, wherein the CPT-1 inhibitor is a compound of the formula (I) as defined herein, wherein
R¹ is unsubstituted or substituted phenyl, naphthyl, or tetrahydronaphthyl,
   unsubstituted or substituted pyridyl, pyrazinyl, pyrimidinyl, oxazolyl, oxydiazolyl, isoxazolyl, thiadiazolyl, tetrazolyl, pyrazolyl, imidazolyl, thiazolyl, thienyl, or quinolinyl,
   unsubstituted or substituted pyrrolidinyl, piperidinyl, tetrahydropiperidinyl, or piperazinyl, or
   unsubstituted or substituted cyclopentyl, cyclohexyl or cyclohexenyl,
   each R¹ being optionally and independently substituted with one or more, preferably with one of the following residues:
   —CN,
   halogen, preferably —F or —Cl,
   C₁-C₄-alkyl, preferably methyl,
   halogen-C₁-C₄-alkyl, preferably difluoromethyl, or trifluoromethyl,
   SO₂Me, or
   CO₂C₁-C₄-alkyl, preferably CO₂Me.

In another embodiment, the invention relates to a Carnitine-Palmitoyl-Transferase-1 (CPT-1) inhibitor for use as defined herein, wherein the CPT-1 inhibitor is a compound of the formula (I) as defined herein, wherein
R¹ is unsubstituted or substituted phenyl,
   unsubstituted or substituted pyridyl, pyrazolyl, thienyl, or quinolinyl,
   unsubstituted or substituted piperidinyl, or tetrahydropiperidinyl, or
   unsubstituted or substituted cyclohexyl or cyclohexenyl;
   each R¹ being optionally and independently substituted with one or more, preferably with one of the following residues:
   —CN,
   —F or —Cl,
   C₁-C₄-alkyl, preferably methyl,
   halogen-C₁-C₄-alkyl, preferably trifluoromethyl,
   SO₂Me, or
   CO₂C₁-C₄-alkyl, preferably CO₂Me.

In another embodiment, the invention relates to a Carnitine-Palmitoyl-Transferase-1 (CPT-1) inhibitor for use as defined herein, wherein the CPT-1 inhibitor is a compound of the formula (I) as defined herein, wherein
R¹ is unsubstituted or substituted phenyl or pyridyl,
   being optionally and independently substituted with one or more, preferably with one of the following residues:
   —F or —Cl,
   C₁-C₄-alkyl, preferably methyl,
   trifluoromethyl,
   SO₂Me, or
   CO₂C₁-C₄-alkyl, preferably CO₂Me.

In another embodiment, the invention relates to a Carnitine-Palmitoyl-Transferase-1 (CPT-1) inhibitor for use as defined herein, wherein the CPT-1 inhibitor is a compound of the formula (I) as defined herein, wherein
L is a single bond, *—CH₂O—, or *—CH₂—, preferably *—CH₂O—, wherein the * indicates the point of attachment to the carbonyl (C═O) group.

In another embodiment, the invention relates to a Carnitine-Palmitoyl-Transferase-1 (CPT-1) inhibitor for use as defined herein, wherein the CPT-1 inhibitor is a compound of the formula (I) as defined herein, wherein
R² is unsubstituted or substituted phenyl, naphthyl, or pyridyl; preferably phenyl,
   each R² being optionally and independently substituted with one or more, preferably with one of the following residues:
   —CN,
   halogen, preferably —F or —Cl,
   C₁-C₄-alkyl, preferably methyl,
   halogen-C₁-C₄-alkyl, preferably difluoromethyl, or trifluoromethyl,
   SO₂Me,
   CO₂C₁-C₄-alkyl, preferably CO₂Me,
   adamantyl,
   unsubstituted or substituted phenyl, being optionally substituted with one or more, preferably with one substituent selected from
   halogen, preferably —F or —Cl,
   halogen-C₁-C₄-alkyl, preferably trifluoromethyl,
   C₃-C₈-cycloalkyl, preferably cyclohexyl, or pyridyl.

In another embodiment, the invention relates to a Carnitine-Palmitoyl-Transferase-1 (CPT-1) inhibitor for use as defined herein, wherein the CPT-1 inhibitor is a compound of the formula (I) as defined herein, wherein
R³ is H, C₁-C₄-alkyl, halogen-C₁-C₄-alkyl, cyclopropyl, cyclobutyl, cyclopentyl or cyclohexyl,
   unsubstituted or substituted azetidinyl, pyrrolidinyl, piperidinyl, or oxetanyl,
   unsubstituted or substituted phenyl.
   each azetidinyl, pyrrolidinyl, piperidinyl, oxetanyl, or phenyl being optionally and independently substituted with one or more, preferably with one of the following residues:
   —CN,
   halogen, preferably —F or —Cl;
   C₁-C₄-alkyl, preferably methyl;
   halogen-C₁-C₄-alkyl, preferably chloromethyl, dichloromethyl, trichloromethyl, fluoromethyl, difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2fluoroethyl, 2-chloro-2,2-difluoroethyl, 2,2-dichloro-2-fluoroethyl, 2,2,2-trichloroethyl, pentafluoroethyl, or 2,2-difluor-3-methyl-butyl,
   particularly preferred difluoromethyl, or trifluoromethyl,
   SO₂Me,
   CO₂C₁-C₄-alkyl, preferably CO₂Me, or
   CO—C₁-C₄-alky, preferably CO-Me.

In another embodiment, the invention relates to a Carnitine-Palmitoyl-Transferase-1 (CPT-1) inhibitor for use as defined herein, wherein the CPT-1 inhibitor is a compound of the formula (I) as defined herein, wherein
R³ is C₁-C₄-alkyl, preferably methyl.

In one embodiment, the invention relates to a Carnitine-Palmitoyl-Transferase-1 (CPT-1) inhibitor for use as defined herein, wherein the CPT-1 inhibitor is a compound of the formula (I) as defined herein, wherein
R¹ is unsubstituted or substituted phenyl,
   unsubstituted or substituted pyridyl, pyrazolyl, thienyl, or quinolinyl,
   unsubstituted or substituted piperidinyl, tetrahydropiperidinyl, or piperazinyl, or
   unsubstituted or substituted cyclopentyl; cyclohexyl or cyclohexenyl;
   each R¹ being optionally and independently substituted with one of the following residues:
   —CN, —F or —Cl, C₁-C₄-alkyl, preferably methyl, or trifluoromethyl,
L is a single bond, *—OCH₂—, or *—CH₂—, or *, wherein the * indicates the point of attachment to the carbonyl (C═O) group;
R² is unsubstituted or substituted phenyl or naphthyl;
   each R² being optionally and independently substituted with one of the following residues:
   —CN, —F or —Cl, C₁-C₄-alkyl, preferably methyl, or trifluoromethyl,
   unsubstituted or substituted phenyl, being optionally substituted with one substituent selected from —F or —Cl, trifluoromethyl, or cyclohexyl, or pyridyl.
R³ is C₁-C₄-alkyl, halogen-C₁-C₄-alkyl, cyclopropyl, cyclobutyl, cyclopentyl or cyclohexyl,
   unsubstituted or substituted azetidinyl, pyrrolidinyl, piperidinyl, oxetanyl, or phenyl;
   each azetidinyl, pyrrolidinyl, piperidinyl, oxetanyl, or phenyl being optionally and independently substituted with one of the following residues: methyl; trifluoromethyl, 2,2-difluoro-3-methyl-butyl, or CO-Me
   and
Y is —(C═O)—.

In another embodiment, the invention relates to a Carnitine-Palmitoyl-Transferase-1 (CPT-1) inhibitor for use as defined herein, wherein the CPT-1 inhibitor is a compound of the formula (I), selected from

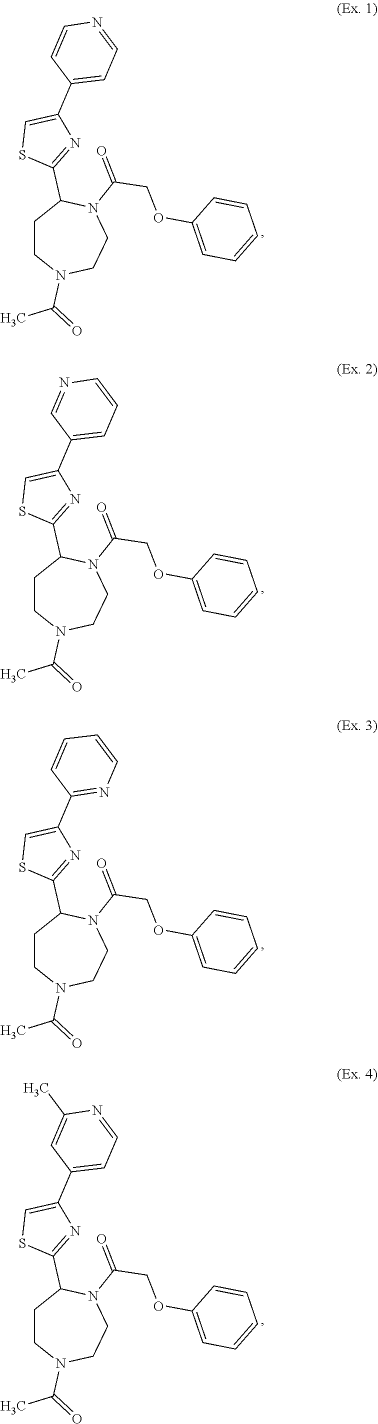 [SMILES: CC(=O)N1CCC(c2nc(-c3ccncc3)cs2)N(C(=O)COc2ccccc2)CC1 ; CC(=O)N1CCC(c2nc(-c3cccnc3)cs2)N(C(=O)COc2ccccc2)CC1 ; CC(=O)N1CCC(c2nc(-c3ccccn3)cs2)N(C(=O)COc2ccccc2)CC1 ; CC(=O)N1CCC(c2nc(-c3ccnc(C)c3)cs2)N(C(=O)COc2ccccc2)CC1]

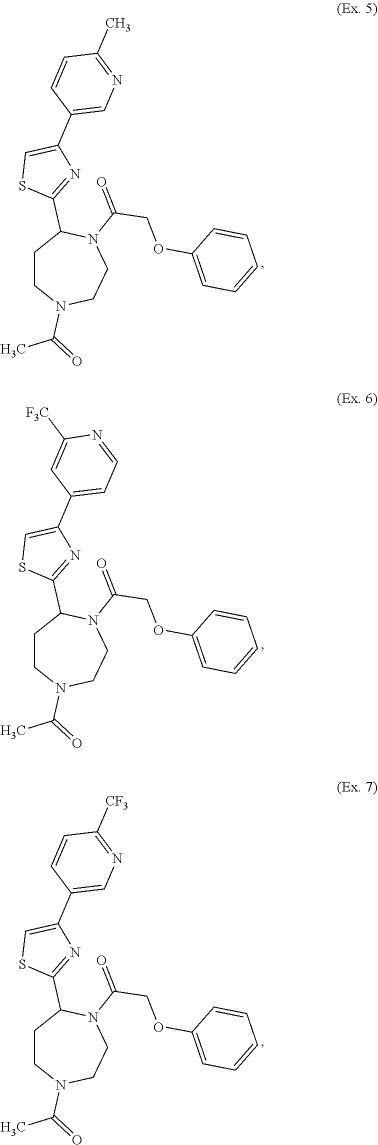 [SMILES: CC(=O)N1CCC(c2nc(-c3ccc(C)nc3)cs2)N(C(=O)COc2ccccc2)CC1 ; CC(=O)N1CCC(c2nc(-c3ccnc(C(F)(F)F)c3)cs2)N(C(=O)COc2ccccc2)CC1 ; CC(=O)N1CCC(c2nc(-c3ccc(C(F)(F)F)nc3)cs2)N(C(=O)COc2ccccc2)CC1]

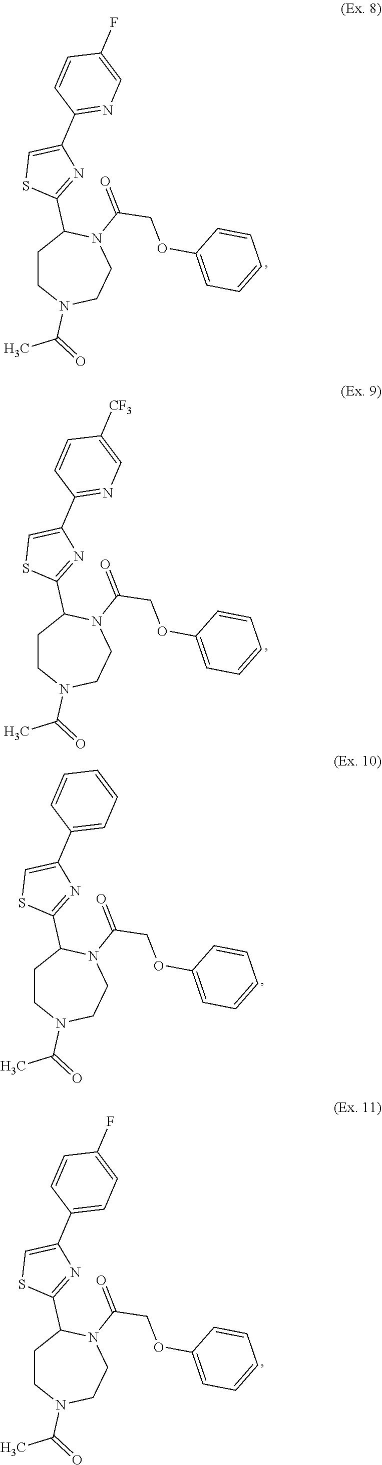

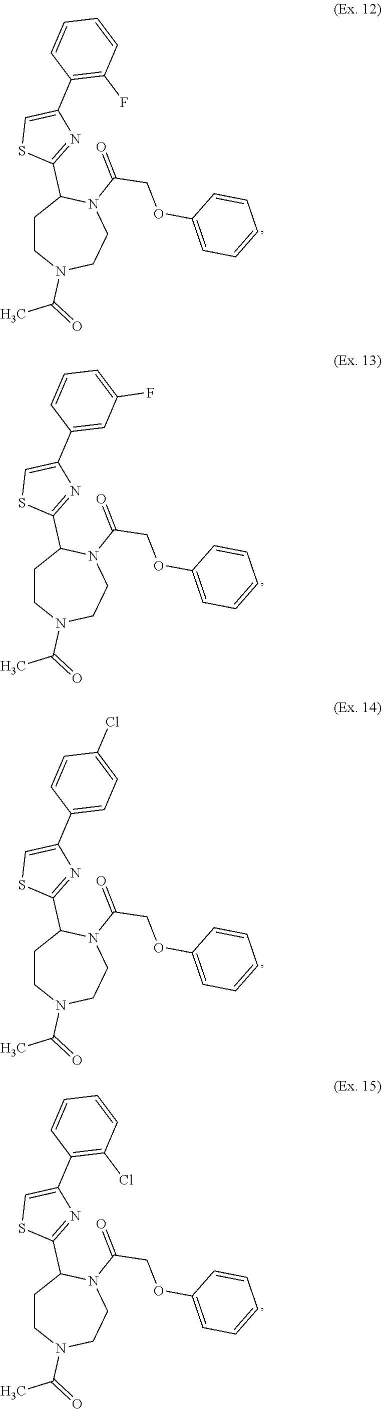

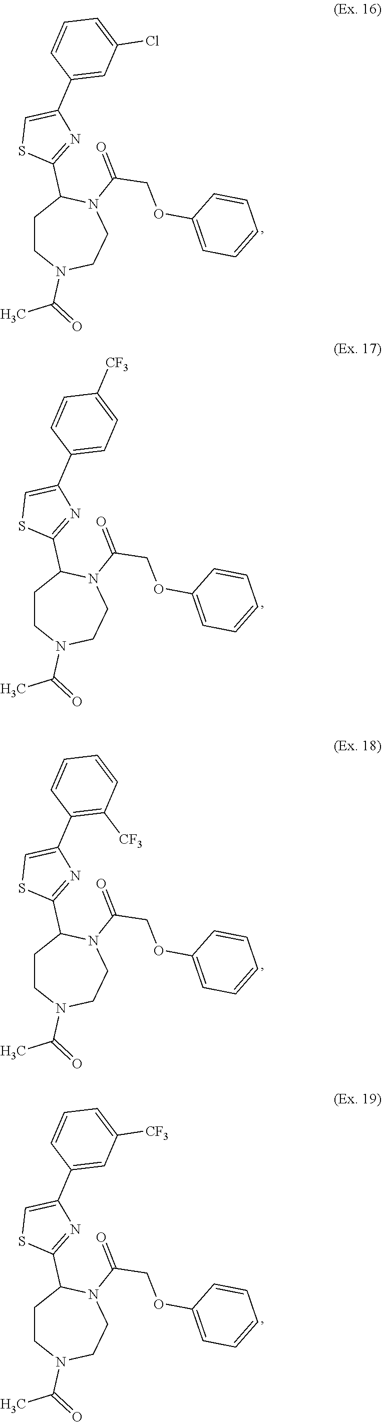 [SMILES: CC(=O)N1CCC(c2nc(-c3cccc(Cl)c3)cs2)N(C(=O)COc2ccccc2)CC1 ; CC(=O)N1CCC(c2nc(-c3ccc(C(F)(F)F)cc3)cs2)N(C(=O)COc2ccccc2)CC1 ; CC(=O)N1CCC(c2nc(-c3ccccc3C(F)(F)F)cs2)N(C(=O)COc2ccccc2)CC1 ; CC(=O)N1CCC(c2nc(-c3cccc(C(F)(F)F)c3)cs2)N(C(=O)COc2ccccc2)CC1]

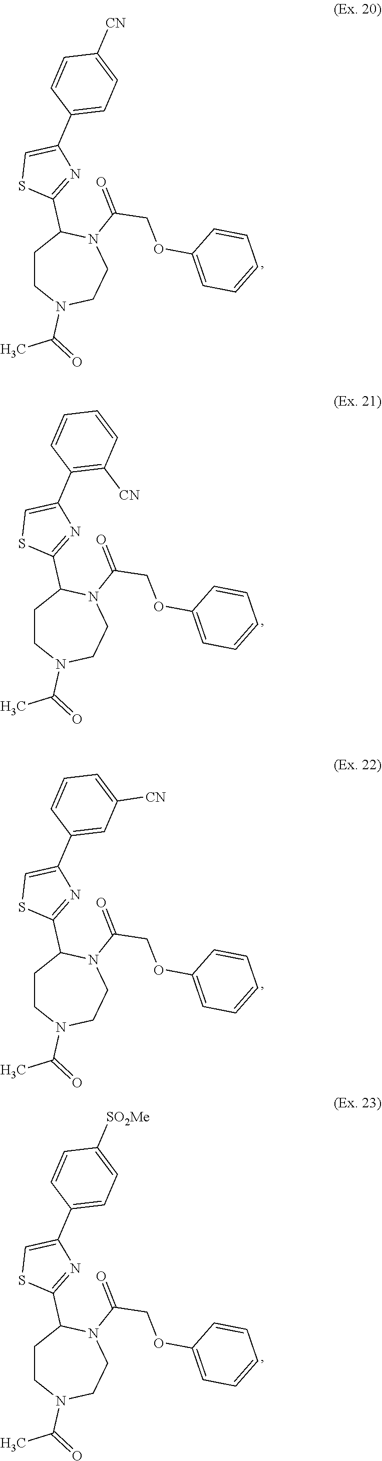 [SMILES: CC(=O)N1CCC(c2nc(-c3ccc(C#N)cc3)cs2)N(C(=O)COc2ccccc2)CC1 ; CC(=O)N1CCC(c2nc(-c3ccccc3C#N)cs2)N(C(=O)COc2ccccc2)CC1 ; CC(=O)N1CCC(c2nc(-c3cccc(C#N)c3)cs2)N(C(=O)COc2ccccc2)CC1 ; CC(=O)N1CCC(c2nc(-c3ccc(S(C)(=O)=O)cc3)cs2)N(C(=O)COc2ccccc2)CC1]

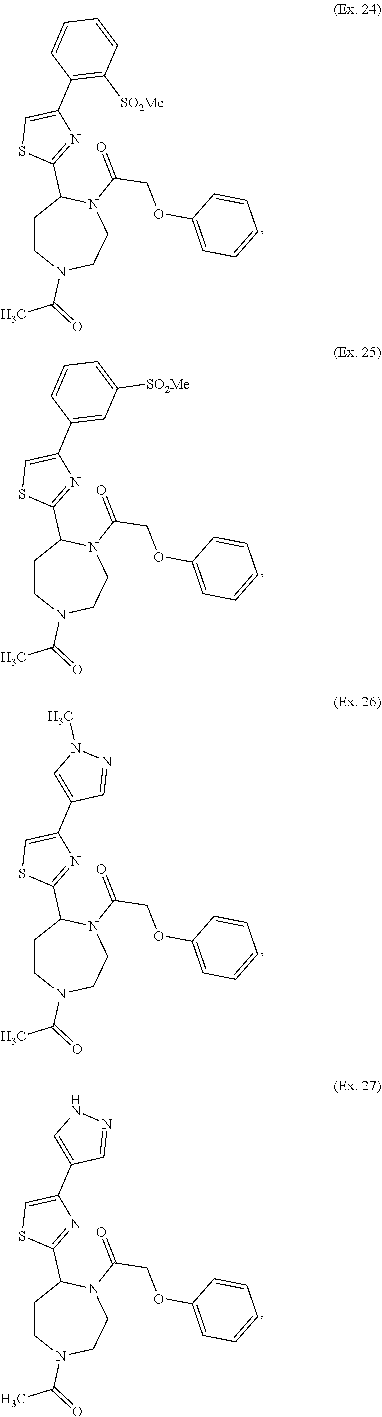

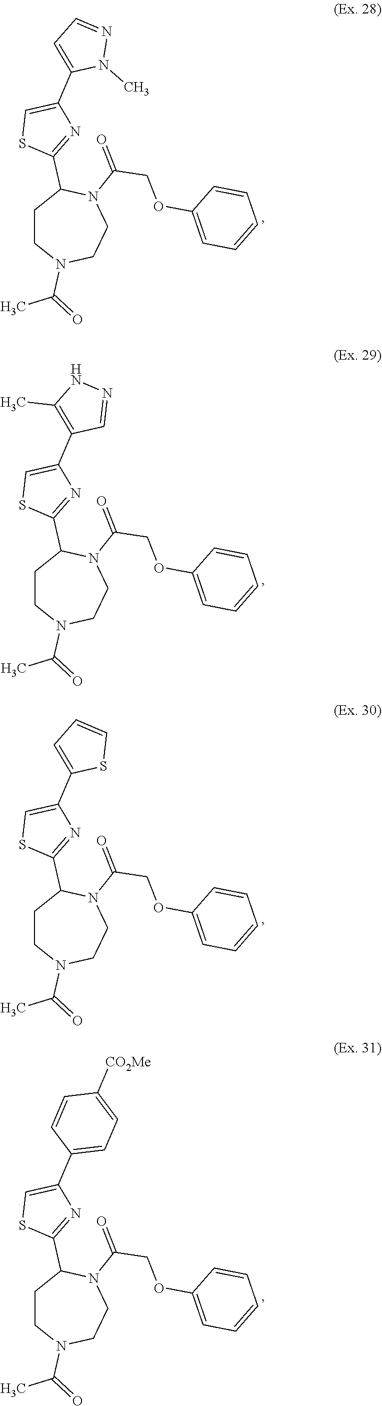

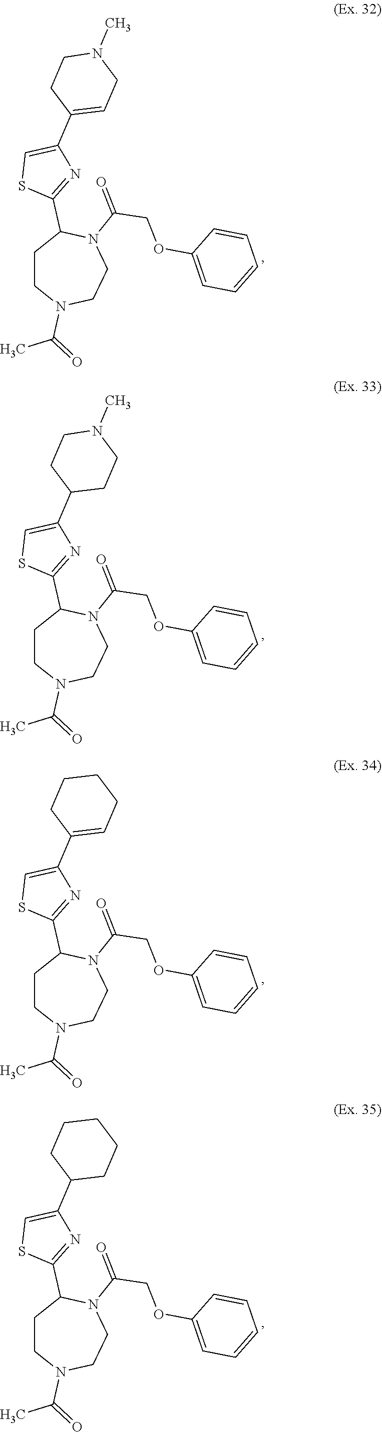

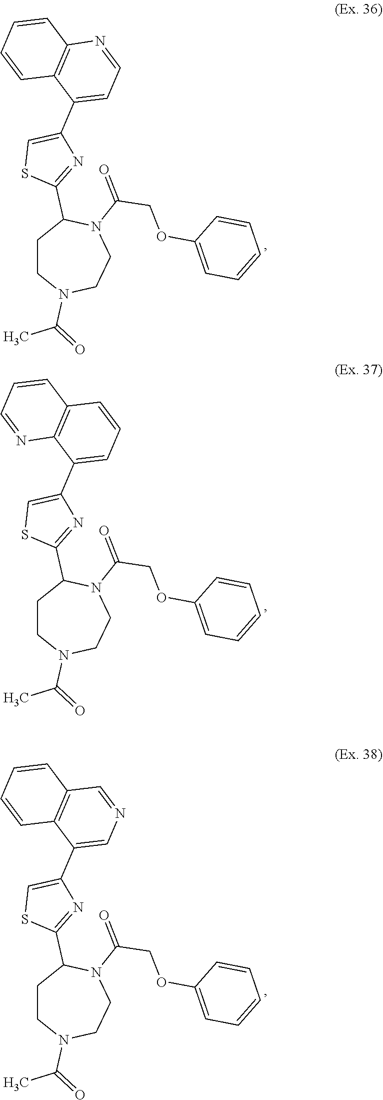

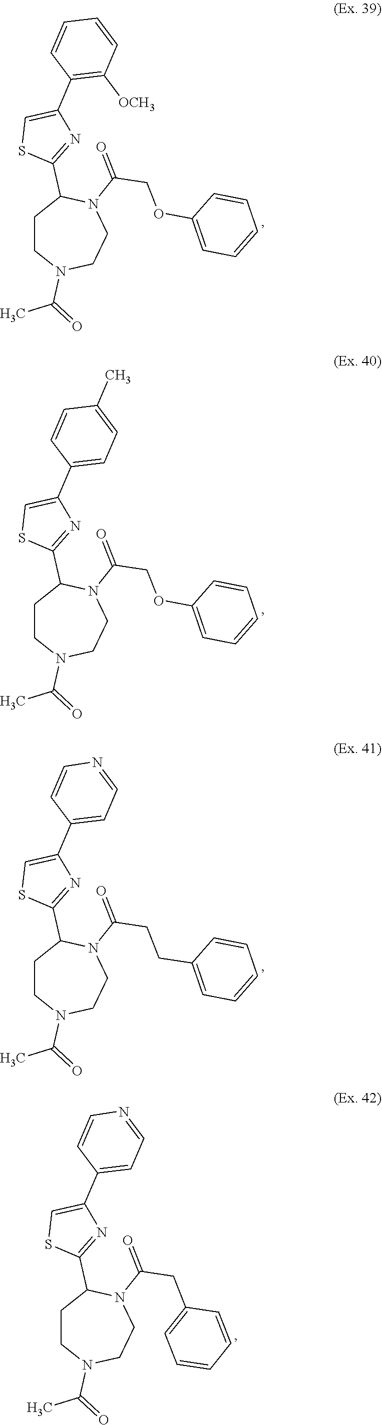 [SMILES: COc1ccccc1-c1csc(C2CCN(C(C)=O)CCN2C(=O)COc2ccccc2)n1 ; CC(=O)N1CCC(c2nc(-c3ccc(C)cc3)cs2)N(C(=O)COc2ccccc2)CC1 ; CC(=O)N1CCC(c2nc(-c3ccncc3)cs2)N(C(=O)CCc2ccccc2)CC1 ; CC(=O)N1CCC(c2nc(-c3ccncc3)cs2)N(C(=O)Cc2ccccc2)CC1]

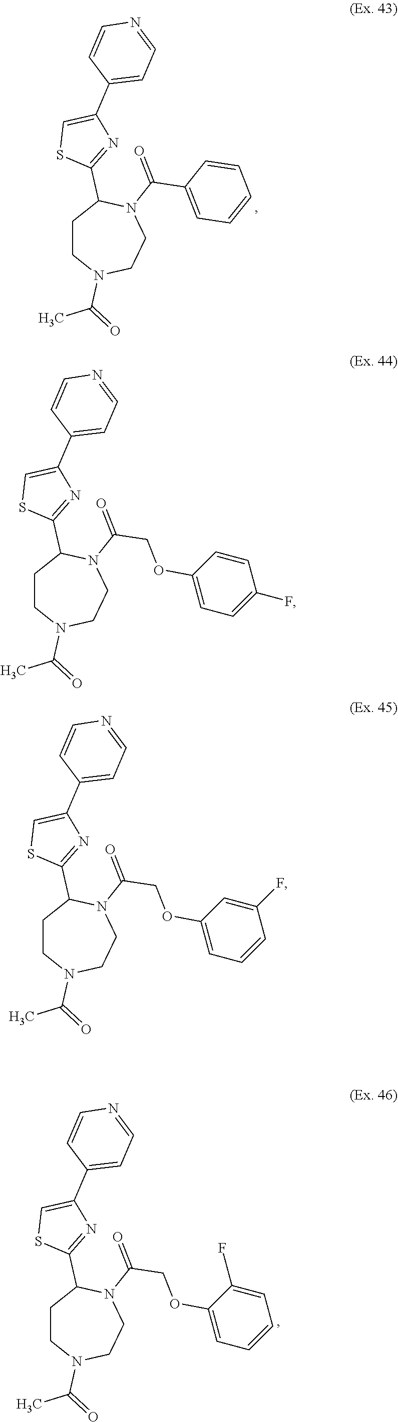

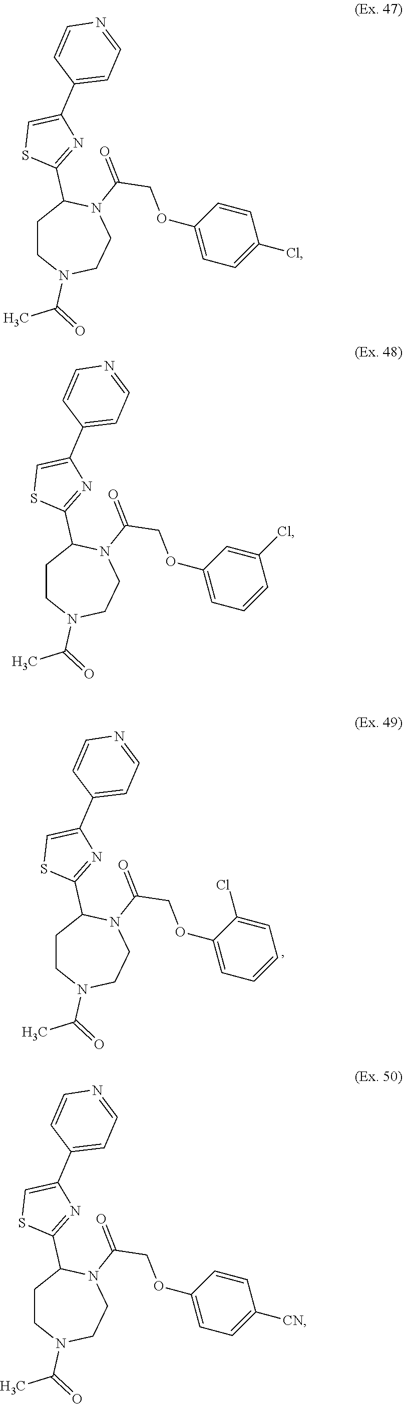

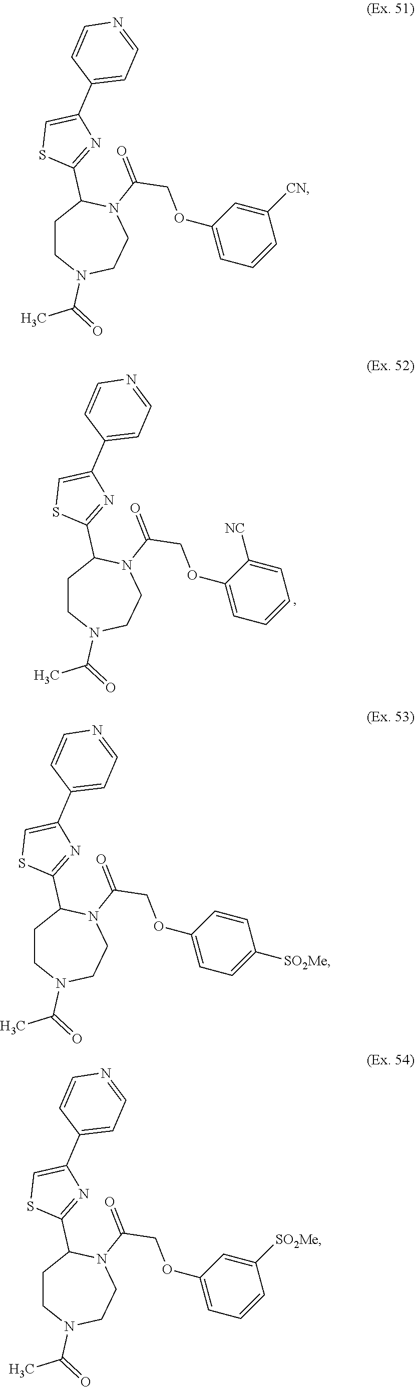

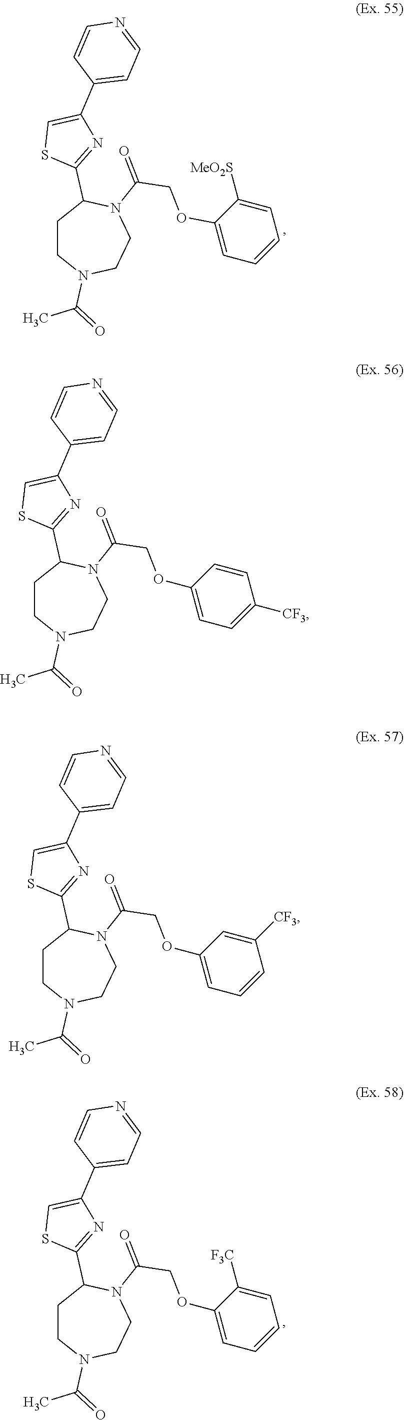 [SMILES: CC(=O)N1CCC(c2nc(-c3ccncc3)cs2)N(C(=O)COc2ccccc2S(C)(=O)=O)CC1 ; CC(=O)N1CCC(c2nc(-c3ccncc3)cs2)N(C(=O)COc2ccc(C(F)(F)F)cc2)CC1 ; CC(=O)N1CCC(c2nc(-c3ccncc3)cs2)N(C(=O)COc2cccc(C(F)(F)F)c2)CC1 ; CC(=O)N1CCC(c2nc(-c3ccncc3)cs2)N(C(=O)COc2ccccc2C(F)(F)F)CC1]

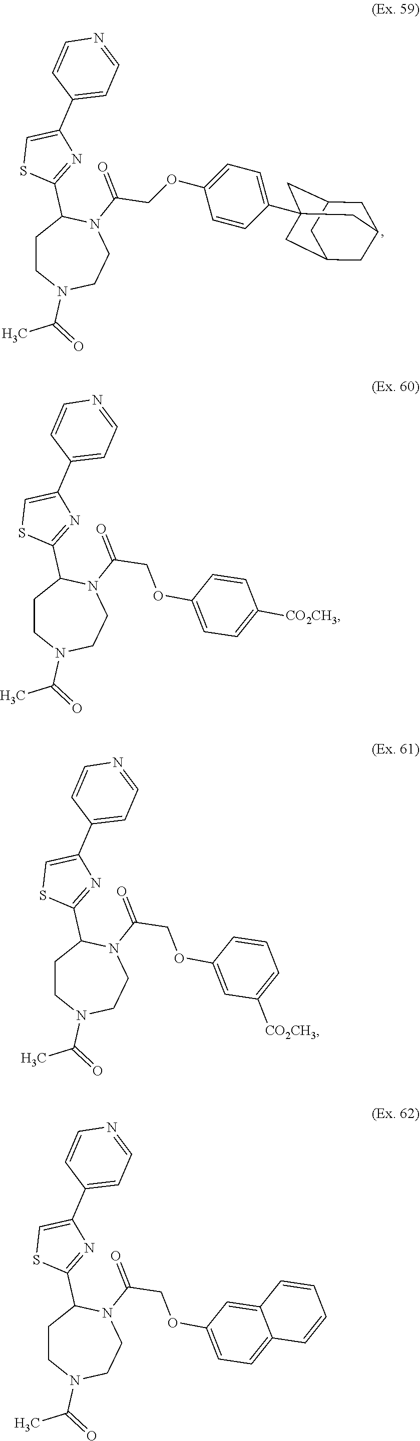 [SMILES: CC(=O)N1CCC(c2nc(-c3ccncc3)cs2)N(C(=O)COc2ccc(C34CC5CC(CC(C5)C3)C4)cc2)CC1 ; COC(=O)c1ccc(OCC(=O)N2CCN(C(C)=O)CCC2c2nc(-c3ccncc3)cs2)cc1 ; COC(=O)c1cccc(OCC(=O)N2CCN(C(C)=O)CCC2c2nc(-c3ccncc3)cs2)c1 ; CC(=O)N1CCC(c2nc(-c3ccncc3)cs2)N(C(=O)COc2ccc3ccccc3c2)CC1]

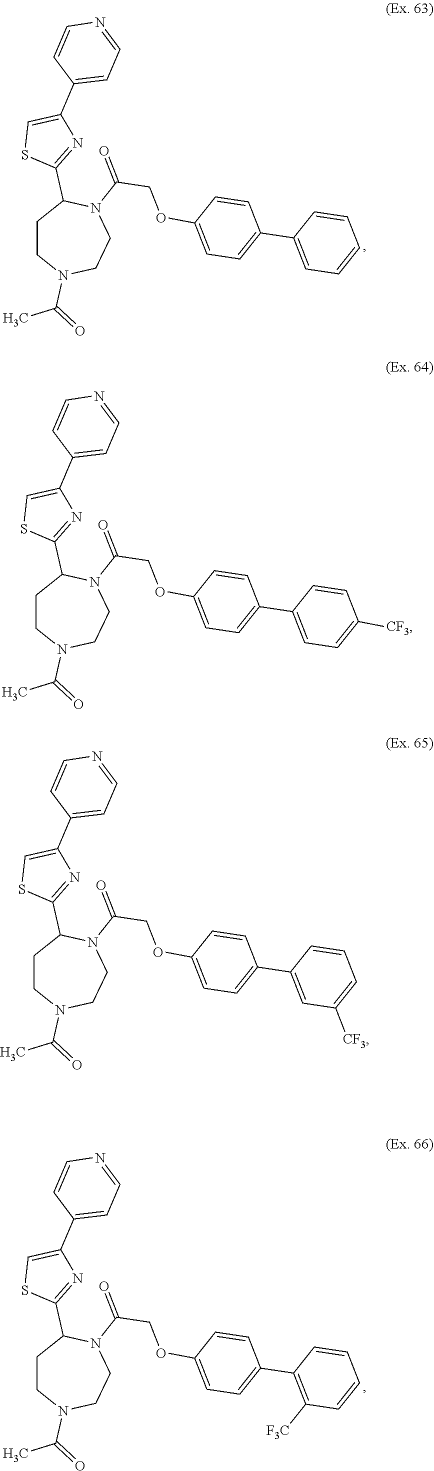 [SMILES: CC(=O)N1CCC(c2nc(-c3ccncc3)cs2)N(C(=O)COc2ccc(-c3ccccc3)cc2)CC1 ; CC(=O)N1CCC(c2nc(-c3ccncc3)cs2)N(C(=O)COc2ccc(-c3ccc(C(F)(F)F)cc3)cc2)CC1 ; CC(=O)N1CCC(c2nc(-c3ccncc3)cs2)N(C(=O)COc2ccc(-c3cccc(C(F)(F)F)c3)cc2)CC1 ; CC(=O)N1CCC(c2nc(-c3ccncc3)cs2)N(C(=O)COc2ccc(-c3ccccc3C(F)(F)F)cc2)CC1]

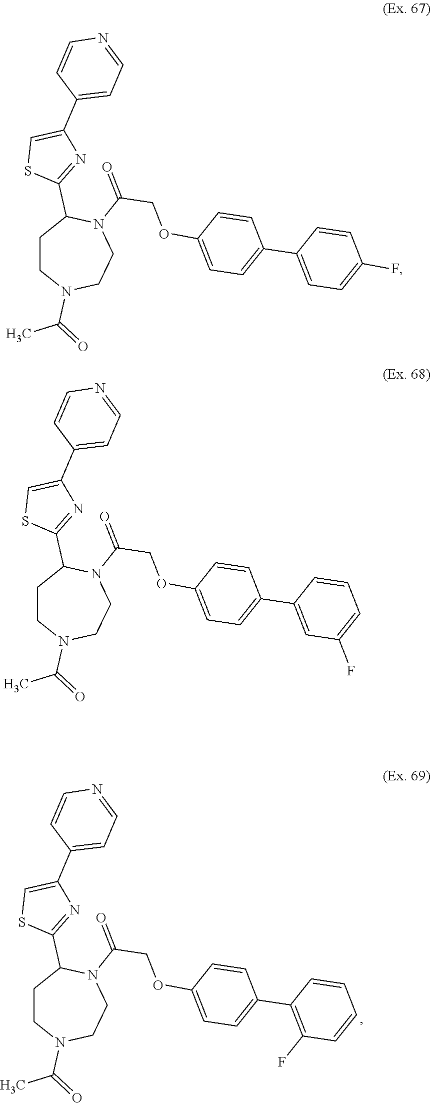

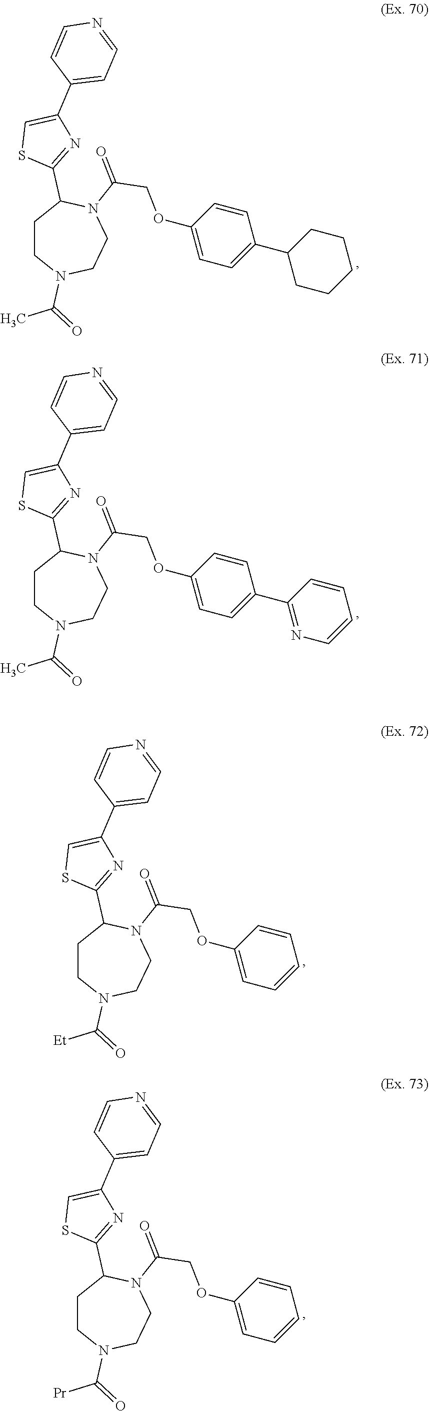

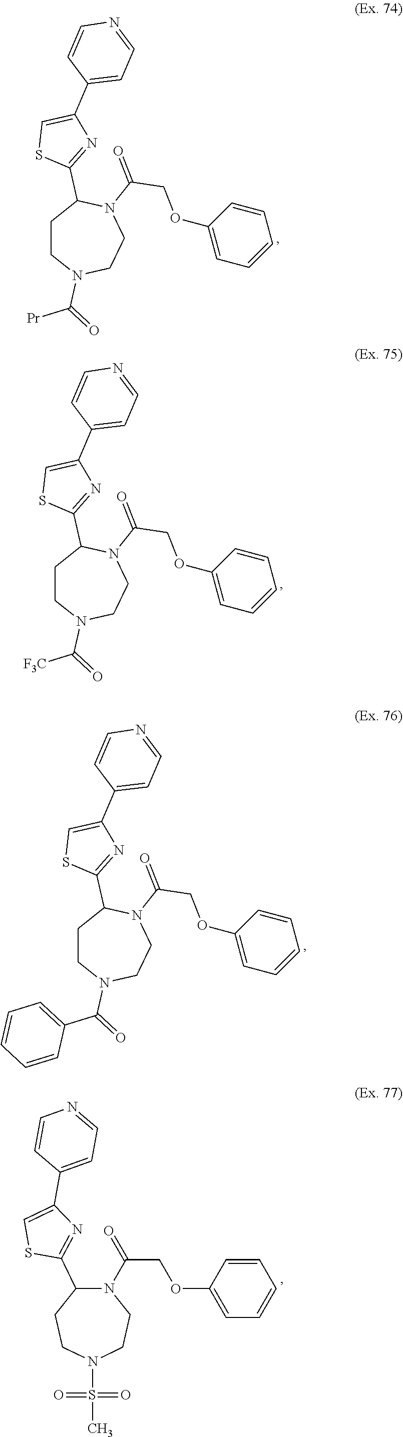

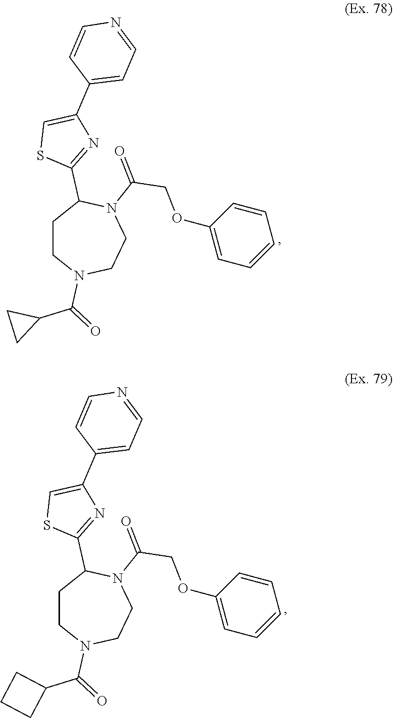 [SMILES: O=C(C1CC1)N1CCC(c2nc(-c3ccncc3)cs2)N(C(=O)COc2ccccc2)CC1 ; O=C(C1CCC1)N1CCC(c2nc(-c3ccncc3)cs2)N(C(=O)COc2ccccc2)CC1]

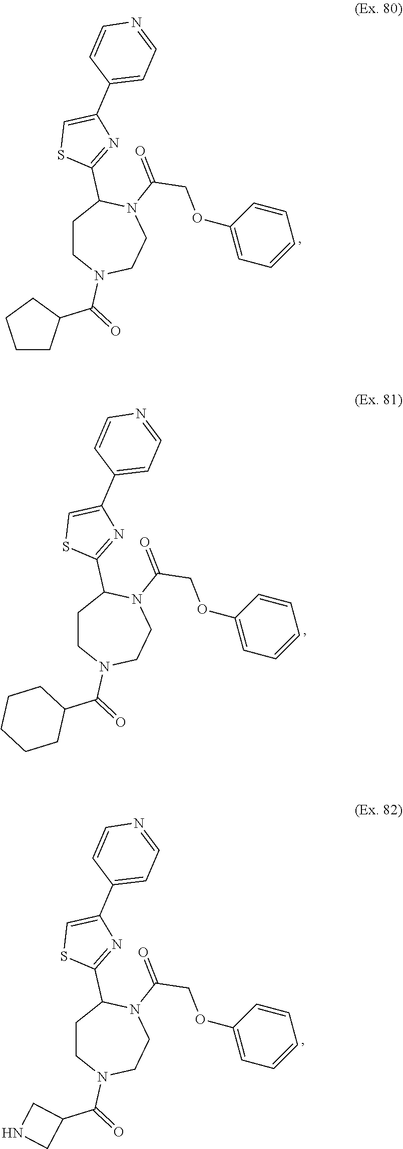 [SMILES: O=C(C1CCCC1)N1CCC(c2nc(-c3ccncc3)cs2)N(C(=O)COc2ccccc2)CC1 ; O=C(C1CCCCC1)N1CCC(c2nc(-c3ccncc3)cs2)N(C(=O)COc2ccccc2)CC1 ; O=C(C1CNC1)N1CCC(c2nc(-c3ccncc3)cs2)N(C(=O)COc2ccccc2)CC1]

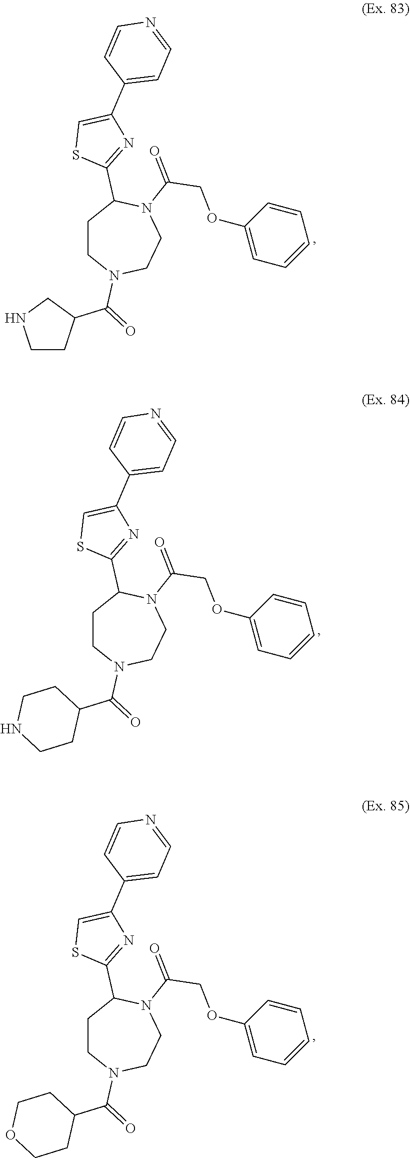 [SMILES: O=C(C1CCNC1)N1CCC(c2nc(-c3ccncc3)cs2)N(C(=O)COc2ccccc2)CC1 ; O=C(C1CCNCC1)N1CCC(c2nc(-c3ccncc3)cs2)N(C(=O)COc2ccccc2)CC1 ; O=C(C1CCOCC1)N1CCC(c2nc(-c3ccncc3)cs2)N(C(=O)COc2ccccc2)CC1]

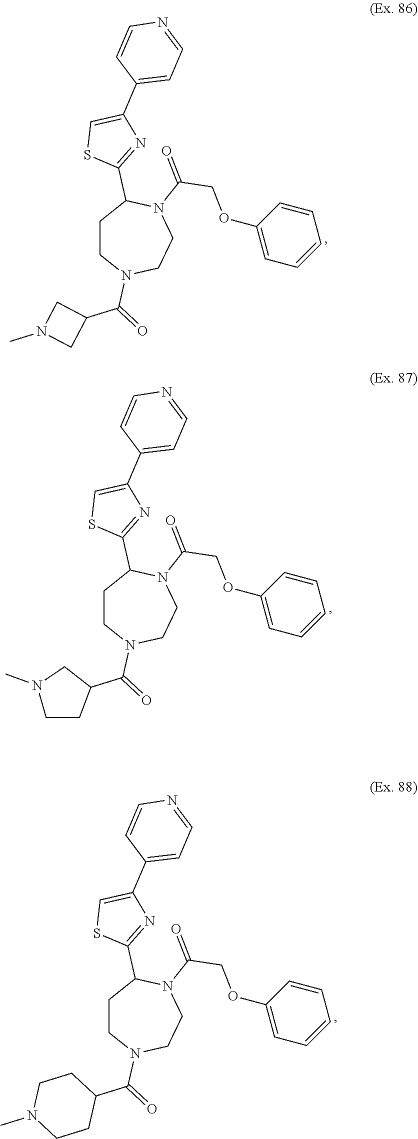 [SMILES: CN1CC(C(=O)N2CCC(c3nc(-c4ccncc4)cs3)N(C(=O)COc3ccccc3)CC2)C1 ; CN1CCC(C(=O)N2CCC(c3nc(-c4ccncc4)cs3)N(C(=O)COc3ccccc3)CC2)C1 ; CN1CCC(C(=O)N2CCC(c3nc(-c4ccncc4)cs3)N(C(=O)COc3ccccc3)CC2)CC1]

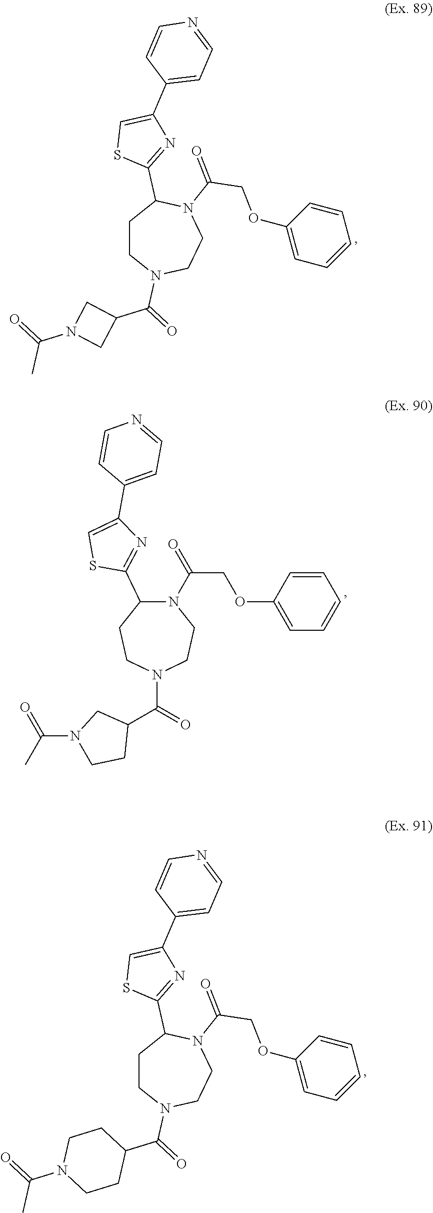 [SMILES: CC(=O)N1CC(C(=O)N2CCC(c3nc(-c4ccncc4)cs3)N(C(=O)COc3ccccc3)CC2)C1 ; CC(=O)N1CCC(C(=O)N2CCC(c3nc(-c4ccncc4)cs3)N(C(=O)COc3ccccc3)CC2)C1 ; CC(=O)N1CCC(C(=O)N2CCC(c3nc(-c4ccncc4)cs3)N(C(=O)COc3ccccc3)CC2)CC1]

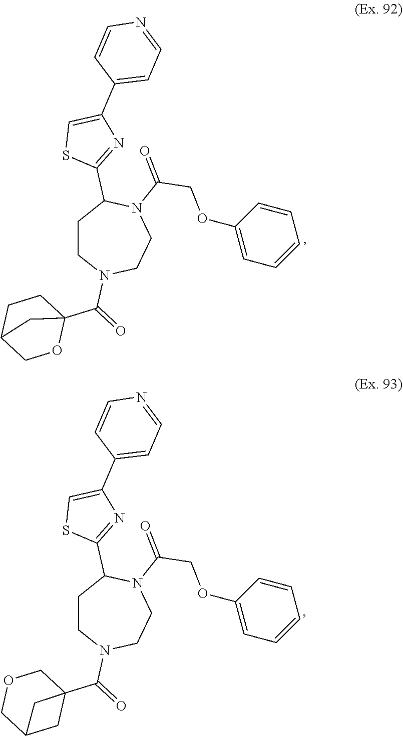 [SMILES: O=C(COc1ccccc1)N1CCN(C(=O)C23CCC(CO2)C3)CCC1c1nc(-c2ccncc2)cs1 ; O=C(COc1ccccc1)N1CCN(C(=O)C23COCC(C2)C3)CCC1c1nc(-c2ccncc2)cs1]

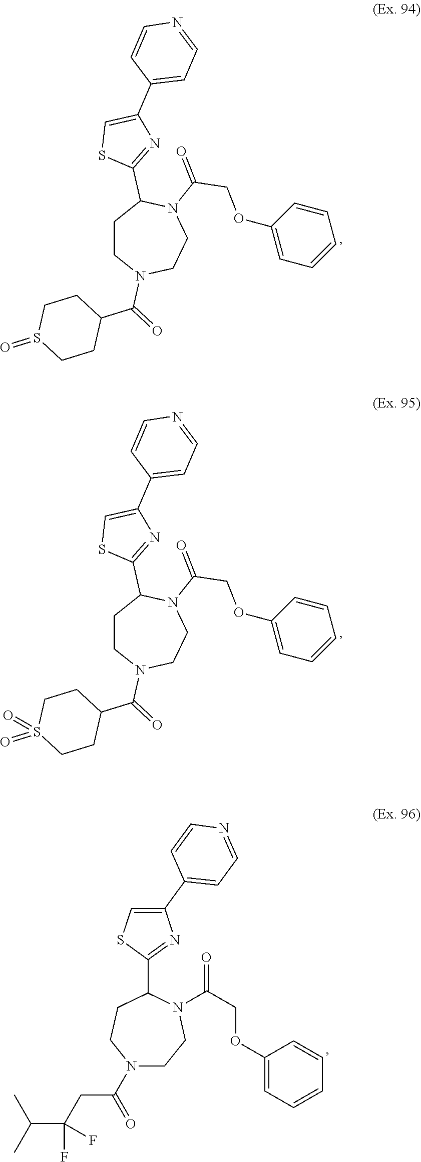 [SMILES: O=C(C1CCS(=O)CC1)N1CCC(c2nc(-c3ccncc3)cs2)N(C(=O)COc2ccccc2)CC1 ; O=C(C1CCS(=O)(=O)CC1)N1CCC(c2nc(-c3ccncc3)cs2)N(C(=O)COc2ccccc2)CC1 ; CC(C)C(F)(F)CC(=O)N1CCC(c2nc(-c3ccncc3)cs2)N(C(=O)COc2ccccc2)CC1]

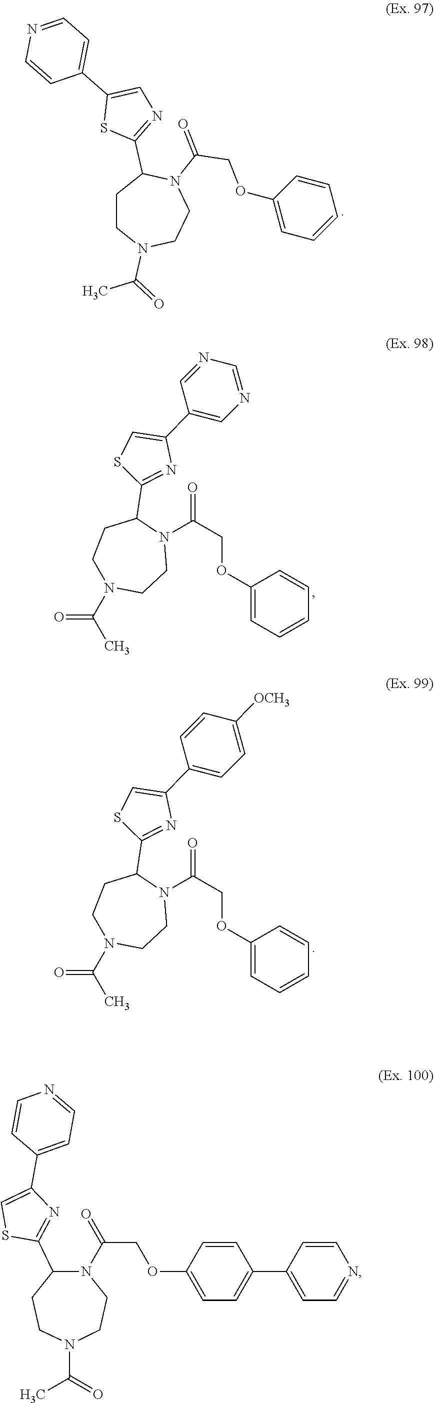 [SMILES: CC(=O)N1CCC(c2ncc(-c3ccncc3)s2)N(C(=O)COc2ccccc2)CC1 ; CC(=O)N1CCC(c2nc(-c3cncnc3)cs2)N(C(=O)COc2ccccc2)CC1 ; COc1ccc(-c2csc(C3CCN(C(C)=O)CCN3C(=O)COc3ccccc3)n2)cc1 ; CC(=O)N1CCC(c2nc(-c3ccncc3)cs2)N(C(=O)COc2ccc(-c3ccncc3)cc2)CC1]

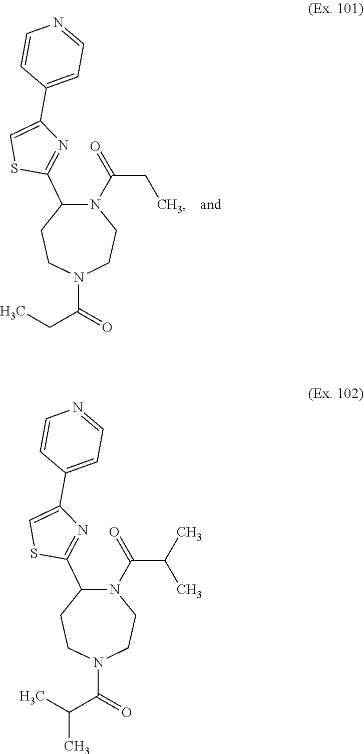 [SMILES: CCC(=O)N1CCC(c2nc(-c3ccncc3)cs2)N(C(=O)CC)CC1 ; "and" ; CC(C)C(=O)N1CCC(c2nc(-c3ccncc3)cs2)N(C(=O)C(C)C)CC1]

In a preferred embodiment, the invention relates to a Carnitine-Palmitoyl-Transferase-1 (CPT-1) inhibitor for use as defined herein, wherein the CPT-1 inhibitor is

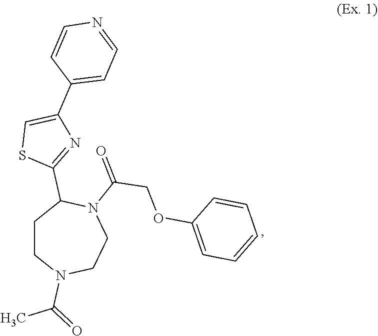 [SMILES: CC(=O)N1CCC(c2nc(-c3ccncc3)cs2)N(C(=O)COc2ccccc2)CC1]

### Processes for the Preparation of Compounds of Formula (I)

The Carnitine-Palmitoyl-Transferase-1 (CPT-1) inhibitors for use as defined herein, wherein the CPT-1 inhibitors are a compound of the formula (I), can be obtained by the processes described below.

A compound of the formula (I) can be obtained by a process comprising the steps of:
(i) reacting a compound of formula (A)

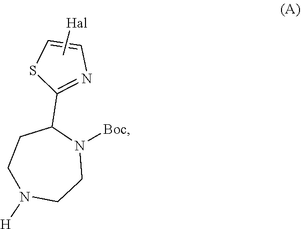

wherein Hal is halogen, preferably Br, and Boc is a tert-butoxycarbonyl protecting group,
with a compound of formula (B)

R³—Y—Cl (B),
wherein R³ and Y are as defined above,
to obtain a compound of formula (C)

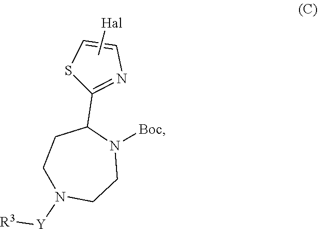

wherein R³, Y, Hal and Boc are as defined above;
(j) reacting the compound of formula (C) with a deprotecting agent to obtain a compound of formula (D)

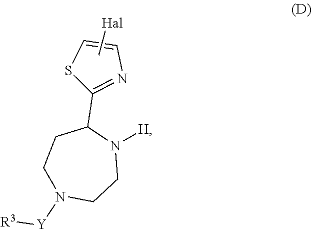

wherein R³, Y and Hal are as defined above;
(k) reacting the compound of formula (D) with a compound of formula (E)

R²-L-CO—Cl (E),
wherein R², and L are as defined above,
to obtain a compound of formula (F)

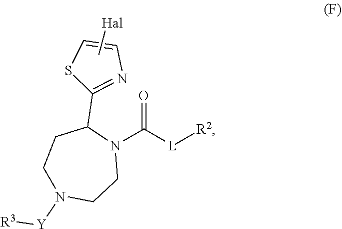

wherein R², R³, L and Y are as defined above;
(l) reacting a compound of formula (F) with a compound of formula (H)

R¹—B(OH)₂ (H),
wherein R¹ is as defined above,
if appropriate in the presence of a catalyst to obtain a compound of formula (I)

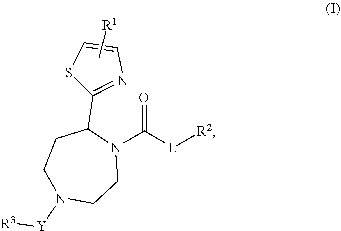

wherein R¹, R², R³, L and Y are as defined above.

An exemplary preparation of preparation of compound of formula (I) is described in Scheme 2 and in the Examples.

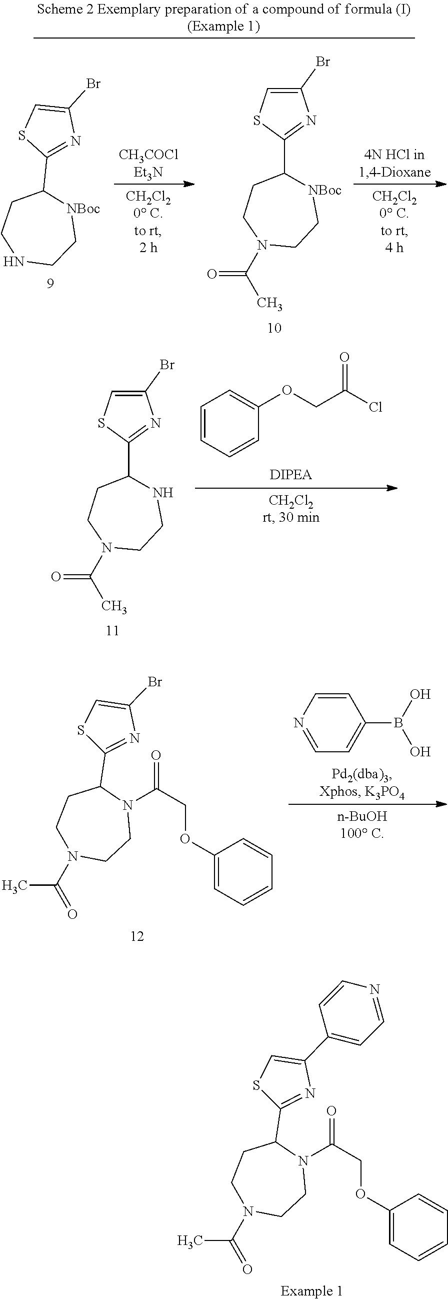

The compounds of formula (A) used as a starting material in process step (i) can be prepared as described in literature procedures or as in the preparation process of the specific Example 9. An exemplary process for the preparation of the compounds of formula (A) is shown in Scheme 1 below:

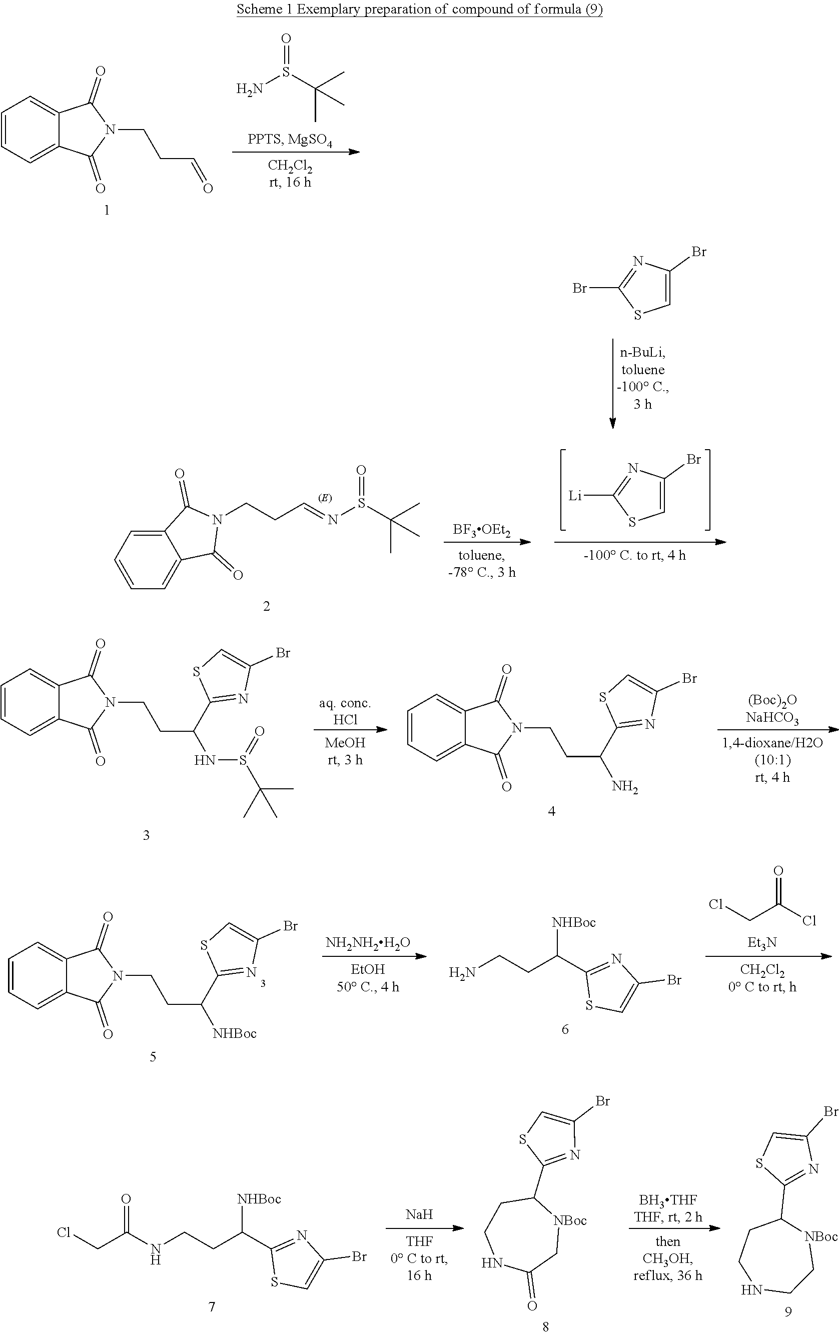

The compounds of formula (B) used as a starting material in process step (i) are commercially available or can be obtained by standard procedures known to the skilled person.

In process step (i), the desired diazepane derivatives of formula (C) may be prepared according to standard N-acetylation procedures known in the art. Further guidance can be found in Scheme 2 and in the Examples disclosed below. Process step (i) is carried out preferably in the present of a solvent and a base, preferably in the presence of dichloromethane and triethylamine.

Process step (j) can be carried out according to standard procedures known in the state of the art for the removal of protecting groups, in particular for removal of the tert-butoxycarbonyl protecting group.

In process step (k), the desired diazepane derivatives of formula (F) may be prepared according to standard N-acetylation procedures known in the art. Further guidance can be found in Scheme 2 and in the Examples disclosed below. Process step (k) is carried out preferably in the present of a solvent and a base, preferably in the presence of dichloromethane and diisopropylethylamine.

The compounds of formula (E) used as a starting material in process step (k) are commercially available or can be obtained by standard procedures known to the skilled person.

Process step (l) can be carried out according to standard procedures known in the state of the art for the C—C-couplings. The compound of formula (I) may be prepared by a standard Suzuki coupling reaction. Further guidance can be found in Scheme 2 and in the Examples disclosed below. Process step (1) is carried out preferably in the present of a solvent and a catalyst.

The compounds of formula (G) used as a starting material in process step (k) are commercially available or can be obtained by standard procedures known to the skilled person.

Alternatively, the compounds of the formula (I) can be obtained by a process comprising the steps shown in Scheme 3.

For example, Examples 2, 4, 5, 7, 10, 12, 17, 23, 26, 27, 40, 98 and 99 of Table 1 (see Table 1—Compounds of formula (I) used in the invention) were obtained according to the process described in Scheme 3. The respective aryl boronic acids Ar—B(OH)₂ used as a starting material are commercially available or can be synthesized according to processes known to the skilled person.

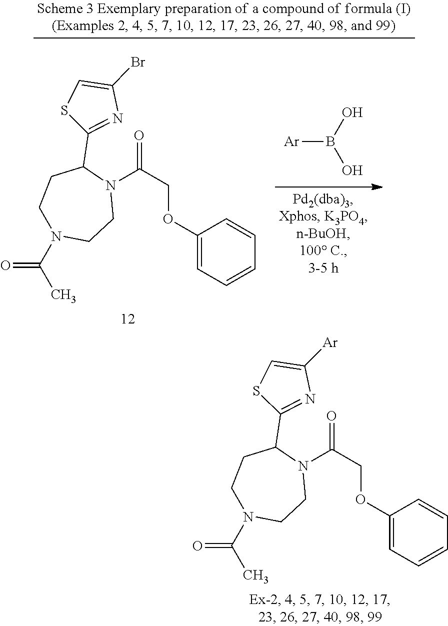

Alternatively, the compounds of the formula (I) can be obtained by a process comprising the steps shown in Scheme 4.

For example, Examples 41-44, 50, 56, 72, 74, 77, 100-102 of Table 1 (see Table 1—Compounds of formula (I) used in the invention) were obtained according to the process described in Scheme 4.

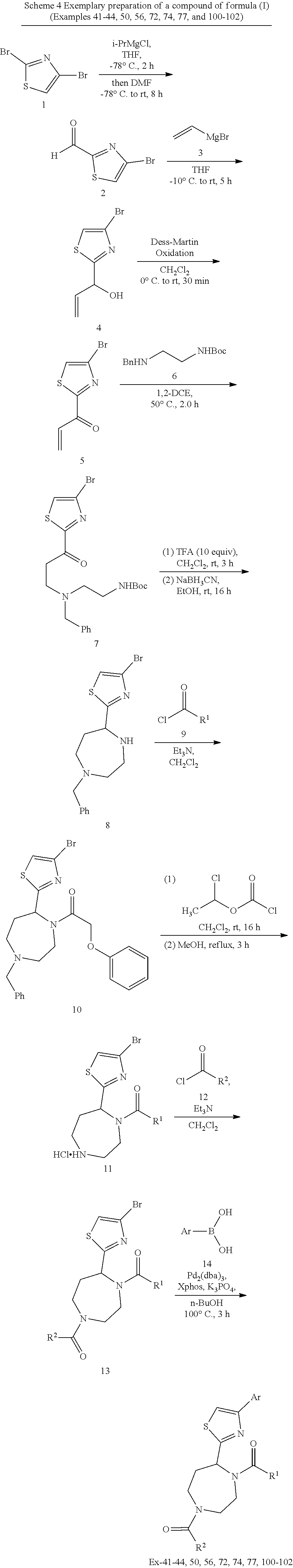

The respective compounds of formulae (9) and (12) and the aryl boronic acids Ar—B(OH)₂ (14) used as a starting material are commercially available or can be synthesized according to processes known to the skilled person.

### Pharmaceutical Composition for Preventing or Treating Sepsis

In another aspect, the invention relates to a pharmaceutical composition comprising the Carnitine-Palmitoyl-Transferase-1 (CPT-1) inhibitor as defined herein for the use as defined herein, further comprising a pharmaceutically acceptable excipient.

As described in the Examples below, CPT-I inhibitors, in particular the compounds of formula (I) used in the invention were surprisingly and unexpectedly shown to be efficient in the fatty acid uptake assay for activity determination using HEK293 cells in vitro (see Example 4.1), and in vivo in an efficacy study in in the CLP-induced mouse model of sepsis (Example 4.2).

Accordingly, CPT-I inhibitors, in particular the compounds of formula (I) used in the invention and their pharmaceutically or veterinarily acceptable salts, hydrates or solvates, exhibit valuable pharmacological properties and are therefore useful for preventing or treating sepsis.

The medicament or pharmaceutical can be further formulated with additional pharmaceutically or veterinary acceptable carriers and/or excipients, e.g. for oral administrations in the form of tablets. Tablets may contain suitable binders, lubricants, disintegrating agents, coloring agents, flavoring agents, flow-inducing agents and/or melting agents, generally known in the art.

The compounds of formula (I) exhibit a marked and selective inhibitory effect on the expression and/or activity of the enzyme Carnitine-Palmitoyl-Transferase-1 (CPT-1). This can be determined for example in an vitro fatty acid uptake assay for activity determination and efficacy study (see Example 4.1). The skilled person however may use different assays to determine the direct or indirect inhibition of CPT-1.

As used herein, the term “pharmaceutically effective amount” of a CPT-I inhibitor means an amount effective to achieve the desired physiological result, either in cells treated in vitro or in a subject treated in vivo. Specifically, a pharmaceutically effective amount is an amount sufficient to inhibit, for some period of time, one or more clinically defined pathological effects associated with disorders caused by delipidation of neural tissue. The pharmaceutically effective amount may vary depending on the specific CPT-I inhibitor selected, and is also dependent on a variety of factors and conditions related to the subject to be treated and the severity of the disease. For example, if the inhibitor is to be administered in vivo, factors such as age, weight, sex, and general health of the patient as well as dose response curves and toxicity data obtained in pre-clinical animal tests would be among the factors to be considered. If the CPT-I inhibitor is to be contacted with cells in vitro, one would also design a variety of pre-clinical in vitro studies to asses parameters like uptake, half-life, dose, toxicity etc. The determination of a pharmaceutically effective amount for a given agent (inhibitor) is well within the ability of those skilled in the art. Preferably, the inhibitor is present in a concentration of 0.01 to 50% per weight of the pharmaceutical composition, more preferably 1 to 30%.

Administration to an individual or patient may be in a single dose or in repeated doses. Repeated doses are preferred, especially once or twice a day until the symptoms disappear or diminish considerably. The patient to be treated with the methods of the present invention is preferably human. However, also animals, preferably mammals as horses, bovines, dogs or cats and more preferably primates can be treated according to the present invention.

The administration of CPT-I inhibitors, in particular of the compound of formula (I) is not limited to a specific route. Preferred routes of administration to an individual include but are not limited to oral systemic, parenteral, especially dermal, intradermal, intracutaneous, percutaneous, subcutaneous, topical or transdermal application. In this context, a systemic application is an application which results in a distribution of the CPT-I inhibitor throughout the body.

### EXAMPLES

### Abbreviations and Acronyms

Abbreviations and Acronyms used in the description of the chemistry and in the Examples that follow are:

| | |
|---|---|
| Boc | tert-butoxycarbonyl |
| CLP | Cecal ligation and puncture-induced |
| CDCl₃ | deuterated chloroform |
| DCM | dichloromethane |
| DIPEA | diisopropylethylamine |
| Ex | Example |
| h | hour |
| ¹H-NMR | ¹H-NMR data of a Compound of formula (I) used |
| | in the invention |
| Isolera | Flash column chromatography (Make: Isolera) |
| LCMS | LCMS data of a Compound of formula (I) used in |
| | the invention |
| Pd₂(dba)₃ | tris(dibenzylidenaceton)dipalladium (0) |
| PPTS | pyridinium p-toluene sulfonate |
| RT | room temperature |
| Structure | structure of a Compound of formula (I) used in the |
| | invention |
| THF | tetrahydrofuran |
| TLC | thin layer chromatography |
| Xphos | 2-dicyclohexylphosphin-2′,4′,6′-triisopropylbiphenyl |

### 1. Experimental Procedures

### 1.1 LCMS Method

Ultra-High-Performance Liquid Chromatography (UHPLC) equipped with SQ 6135 (from Agilent)/SQ 2020 (from Shimadzu) Mass spectrometer and

Electro Spray and Atmospheric pressure chemical ionization source (Multimode source with ESI/APCI).
Column: Column Zorbax Eclipse PlusC18 (50×2.1 mm) 1.8 mp (for Formic Acid Method), or Acquity BEH C18 (2.1×50) mm, 1.7 mp (for Ammonium Bicarbonate Method)
Flow: 0.800 mL/min or 0.600 mL/min
Eluents: A: H₂O with 0.05% formic acid and B: MeCN or A: H₂O with 10 mM Ammonium Bicarbonate B: MeCN
Gradient: Elution from 5% to 100% B over 2.5 min with an initial hold for 0.5 min and a final hold at 95% B of 1.0 min. Total run time: 4 min.
   The gradient described could be altered in the function of the physico-chemical properties of the compound analyzed and is in no way restrictive.

HPLC-Purity were obtained using Shimadzu Instrument
Column: X-Select C18 (4.6×150 mm, 5 μm) or
   X-Bridge column C8 (4.6×150 mm, 5 μm)
Flow: 0.800 mL/min or 0.600 mL/min
Eluents: A: H₂O with 0.05% formic acid and B: MeCN or
   A: H₂O with 0.05% Ammonium Bicarbonate B: MeCN
Gradient: Elution from 5% to 100% B over 8 min then hold at 5% B of 2 min. Total run time: 10 min.
   The gradient described could be altered in function of the physico-chemical properties of the compound analyzed and is in no way restrictive.

### 1.2 NMR Methods

Proton (¹H) nuclear magnetic resonance (NMR) spectras are measured with an Avance Neo Nanobay (400 MHz) spectrometer with residual protonated solvent (CDCl₃ δ 7.28; CD₃OD δ 3.31 and DMSO δ 2.50) as standard. The NMR data of the synthesized examples are in agreement with their corresponding structural assignments.

### 2. Process for the Preparation of the Compounds of Formula (I)

An exemplary synthesis of a compound of formula (I) is described below in Schemes 1 and 2. The compounds of formula (I) used in the invention can be obtained according to the process described in Schemes 1 and 2.

The starting materials are either commercially available or are prepared in similar manners as described in literature procedures or in the specific example.

It should be apparent to those skilled in the art that the sequence of the synthetic steps is dependent on starting materials availability and functional group compatibility and could vary from compound to compound.

### 3. Examples of the Compounds of Formula (I) Used in the Invention

The following Examples are merely specific embodiments of the present invention and are intended to illustrate but not to limit the invention.

### 3.1 Preparation of Intermediates for the Preparation of Compounds of Formula (I)

### 3.1.1 Preparation of tert-butyl 7-(4-bromothiazol-2-yl)-1,4-diazepane-1-carboxylate (9)

Tert-butyl 7-(4-bromothiazol-2-yl)-1,4-diazepane-1-carboxylate (9) was obtained according to the process described in Scheme 1.

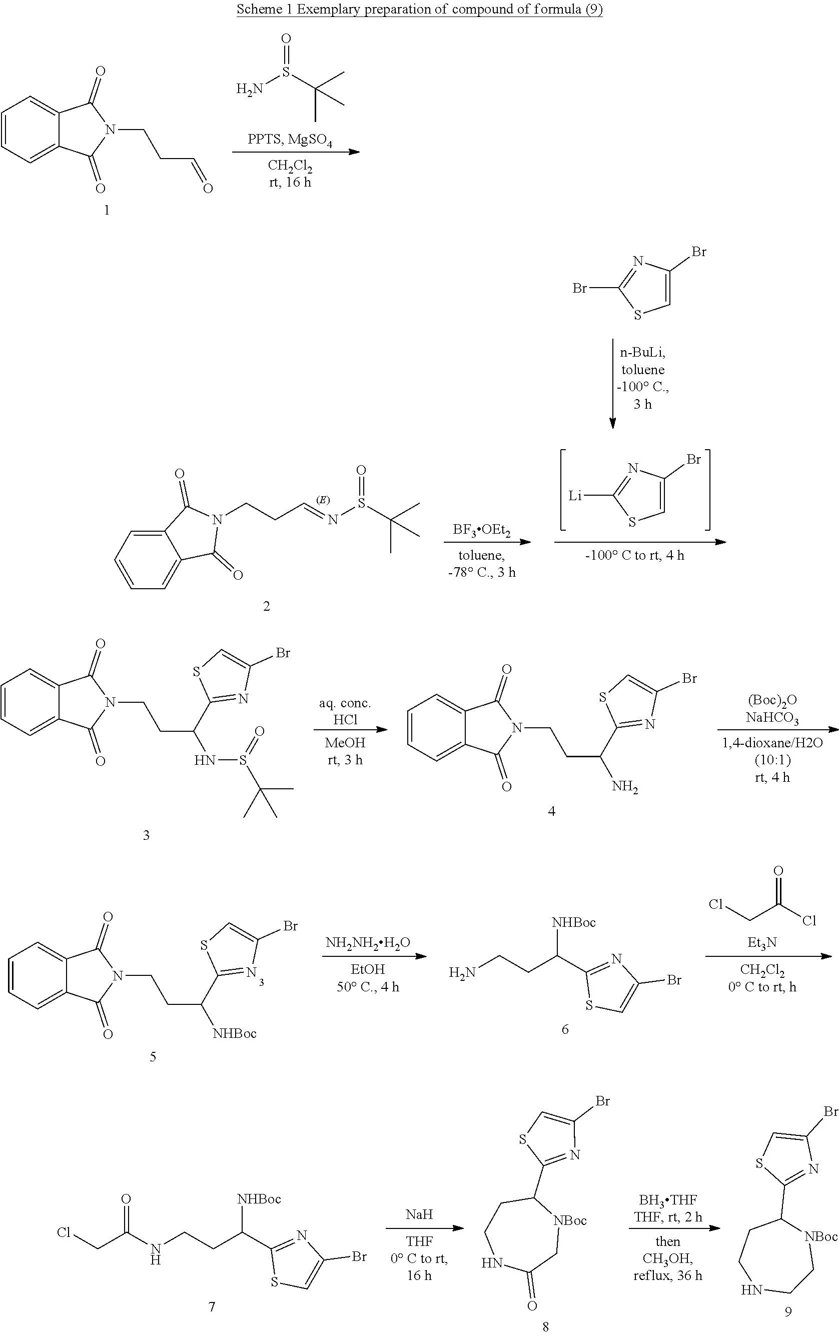

### Step (a): Preparation of (E)-N-(3-(1,3-dioxoisoindolin-2-yl)propylidene)-2-methylpropane-2-sulfinamide (2)

To a stirred solution of 3-(1,3-dioxoisoindolin-2-yl)propanal (1) (14 g, 68.9 mmol) in anhydrous DCM (200 mL) 2-methylpropane-2-sulfinamide (9.1 g, 75.8 mmol) was added at RT. PPTS (0.86 g, 3.4 mmol) and anhydrous magnesium sulfate (41 g, 344 mmol) were added and the mixture was stirred at RT for 16 h. The reaction was monitored by TLC. The mixture was filtered through celite. The filtrate was concentrated to get the crude which was purified by silica column chromatography using Isolera by eluting with 30% ethyl acetate in pet. ether to afford (E)-N-(3-(1,3-dioxoisoindolin-2-yl)propylidene)-2-methylpropane-2-sulfinamide (2) (15 g, yield: 71%). ¹H-NMR (400 MHz, CDCl₃): δ 8.14 (t, J=3.60 Hz, 1H), 7.89-7.87 (m, 2H), 7.77-7.74 (m, 2H), 4.11-4.02 (m, 2H), 3.01-2.96 (m, 2H), 1.19 (s, 9H), LCMS: 307 (M+1).

### Step (b): Preparation of: 2-(3-amino-3-(4-bromothiazol-2-yl) propyl) isoindoline-1,3-dione (3)

To a stirred solution of 2,4-dibromothiazole (23.7 g, 97.97 mmol) in anhydrous toluene (150 mL) n-BuLi (1.6 M in THF, 61.2 mL, 97.97 mmol) was added dropwise at −100° C. The mixture was stirred with a mechanical stirrer at the same temperature for 3 h.

In an another set up, to a solution of (E)-N-(3-(1,3-dioxoisoindolin-2-yl) propylidene)-2-methylpropane-2-sulfinamide (2) (15 g, 48.98 mmol) in anhydrous toluene (100 mL) boron trifluoride diethyl etherate (13.8 mL) was added dropwise at −78° C. and stirred for 3 h. After 3 h, this mixture was cannulated dropwise into the above mixture containing 2,4-dibromothiazole and n-butyl lithium at −100° C. The resulting mixture was allowed to slowly warm to RT and stirred for 4 h. The reaction was monitored by TLC. The reaction was cooled to 0° C. then quenched by the slow addition of ice-cooled water (100 mL). The mixture was extracted with ethyl acetate (2×500 m L). The combined organic layer was dried over anhydrous sodium sulphate, filtered and concentrated. The crude was purified by silica column chromatography using Isolera by eluting with 40% ethyl acetate in pet. ether to afford 8 g of (67% pure by LCMS) N-(1-(4-bromothiazol-2-yl)-3-(1,3-dioxoisoindolin-2-yl)propyl)-2-methylpropane-2-sulfinamide (3). ¹H-NMR (400 MHz, CDCl₃): δ 7.77-7.75 (m, 2H), 7.70-7.68 (m, 2H), 6.85 (s, 1H), 5.18 (d, J=9.60 Hz, 1H), 4.88-4.84 (m, 1H), 4.07-3.99 (m, 1H), 3.96-3.90 (m, 1H), 2.98-2.90 (m, 1H), 2.52-2.45 (m, 1H), 1.41 (s, 9H). LCMS: 470 & 472 (M+1).

### Step (c): Preparation of 2-(3-amino-3-(4-bromothiazol-2-yl)propyl)isoindoline-1,3-dione (4)

To a solution of N-(1-(4-bromothiazol-2-yl)-3-(1,3-dioxoisoindolin-2-yl)propyl)-2-methylpropane-2-sulfinamide (3) (8 g) in MeOH (50 mL) conc. HCl (8 mL) was added, and the mixture was stirred at RT for 3 h. The reaction was monitored by TLC. The mixture was concentrated under reduced pressure to afford 7 g of 2-(3-amino-3-(4-bromothiazol-2-yl)propyl)isoindoline-1,3-dione (4).

### Step (d): Preparation of tert-butyl (1-(4-bromothiazol-2-yl)-3-(1,3-dioxoisoindolin-2-yl)propyl)carbamate (5)

To a stirred solution of 2-(3-amino-3-(4-bromothiazol-2-yl)propyl)isoindoline-1,3-dione (4) (5 g, 13.66 mmol) in dioxane/water (55 mL, 10:1) sodium bicarbonate (7.1 g, 68.3 mmol) was added at RT. Di-tert-butyl dicarbonate (5.9 mL, 27.3 mmol) was added and the mixture was stirred at RT for 4 h. The reaction was monitored by TLC. The mixture was diluted with water (100 mL) and extracted with ethyl acetate (3×100 mL). The combined organic layer was dried over anhydrous sodium sulphate, filtered and concentrated. The crude was purified by silica column chromatography using Isolera by eluting with 15% to 20% ethyl acetate in pet. ether to afford tert-butyl (1-(4-bromothiazol-2-yl)-3-(1,3-dioxoisoindolin-2-yl)propyl)carbamate (5) (2.4 g, yield: 38%). ¹H-NMR (400 MHz, CDCl₃): b 7.84-7.82 (m, 2H), 7.75-7.71 (m, 2H), 7.04 (s, 1H), 5.60-5.58 (m, 1H), 5.15-5.13 (m, 1H), 3.90-3.85 (m, 2H), 2.46-2.45 (m, 2H), 1.48 (s, 9H), LCMS: 466 & 468 (M+1).

### Step (e): Preparation of tert-butyl (3-amino-1-(4-bromothiazol-2-yl)propyl)carbamate (6)

To a stirred solution of tert-butyl (1-(4-bromothiazol-2-yl)-3-(1,3-dioxoisoindolin-2-yl)propyl)carbamate (5) (2.4 g, 5.15 mmol) in ethanol (20 mL) hydrazine hydrate (0.5 mL, 10.3 mmol) was added. The mixture was heated at 50° C. for 4 h. The reaction was monitored by TLC and the solid was filtered through celite and the filtrate was concentrated. The crude was purified by silica column chromatography using Isolera by eluting with 10% MeOH in DCM to afford tert-butyl (3-amino-1-(4-bromothiazol-2-yl)propyl)carbamate (6) (1.5 g, 81% pure). LCMS: 336 & 338 (M+1).

### Step (f): Preparation of tert-butyl (1-(4-bromothiazol-2-yl)-3-(2-chloroacetamido)propyl)carbamate (7)

To a stirred solution of tert-butyl (3-amino-1-(4-bromothiazol-2-yl)propyl)carbamate (6) (1.5 g, 4.46 mmol) in anhydrous DCM (20 mL) triethylamine (0.9 mL, 6.69 mmol) was added at 0° C. Chloroacetyl chloride (0.53 mL, 6.69 mmol) was added dropwise to the mixture and stirred for 4 h. The reaction was monitored by TLC and the mixture was diluted water (50 mL) and DCM (100 mL). The organic layer was separated and washed with 10% aqueous solution of sodium bicarbonate (50 mL×2) and brine (50 mL). The organic layer was dried over anhydrous sodium sulphate, filtered and concentrated to afford tert-butyl (1-(4-bromothiazol-2-yl)-3-(2-chloroacetamido)propyl)carbamate (7) (1.7 g crude). LCMS: 412 & 414 (M+1).

### Step (g): Preparation of tert-butyl 7-(4-bromothiazol-2-yl)-3-oxo-1,4-diazepane-1-carboxylate (8)

To an ice-cooled solution of tert-butyl (1-(4-bromothiazol-2-yl)-3-(2-chloroacetamido)propyl)carbamate (7) (1.7 g, 4.12 mmol) in anhydrous THE (200 mL) sodium hydride (60% dispersion in mineral oil, 0.95 g, 23.8 mmol) was added in three portions and the mixture was stirred at RT for 16 h. The reaction was quenched with ice-cooled water (200 mL) and extracted with ethyl acetate (3×100 mL). The combined organic layer was dried over anhydrous sodium sulphate, filtered and concentrated. The crude was purified by silica column chromatography using Isolera by eluting with 65% ethyl acetate in pet. ether to afford tert-butyl 7-(4-bromothiazol-2-yl)-3-oxo-1,4-diazepane-1-carboxylate (8) (0.2 g, yield: 13%). LCMS: 322 (M+1, t-butyl cleaved).

### Step (h): Preparation of tert-butyl 7-(4-bromothiazol-2-yl)-1,4-diazepane-1-carboxylate (9)

To an ice-cooled solution of tert-butyl 7-(4-bromothiazol-2-yl)-3-oxo-1,4-diazepane-1-carboxylate (8) (0.2 g, 0.531 mmol) in anhydrous THE (5 mL) a 1M solution of BH₃·THF (1.59 mL, 1.59 mmol) was added. The mixture was stirred at RT for 2 h. The reaction was quenched with water (5 m L) and methanol (5 m L). The mixture was heated at 70° C. for 36 h for cleaving the borane complex. The mixture was diluted ice-cooled water (10 mL) and extracted with DCM (2×10 mL). The combined organic layer was dried over anhydrous sodium sulphate, filtered and concentrated to afford tert-butyl 7-(4-bromothiazol-2-yl)-1,4-diazepane-1-carboxylate (9) (0.18 g, yield: 93%). LCMS: 362 & 364 (M+1).

### 3.2 Preparation of Compounds of Formula (I)

Example 1 was obtained according to the process described in Scheme 2.

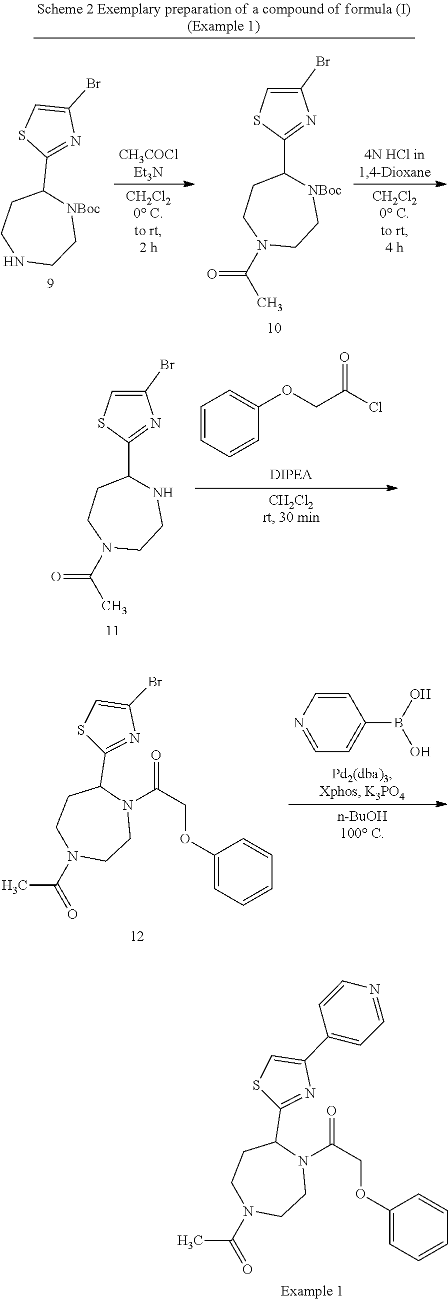

### Step (i): Preparation of tert-butyl 4-acetyl-7-(4-bromothiazol-2-yl)-1,4-diazepane-1-carboxylate (10)

To a stirred solution of tert-butyl 7-(4-bromothiazol-2-yl)-1,4-diazepane-1-carboxylate (9) (0.18 g, 0.497 mmol) in DCM (5 mL) triethylamine (0.2 mL, 1.49 mmol) was added. Acetyl chloride (0.047 g, 0.596 mmol) was added at 0° C. and the mixture was stirred at RT for 2 h. The reaction was monitored by TLC and then quenched with water (10 mL) and extracted with DCM (2×10 mL). The combined organic layer was dried over anhydrous sodium sulphate, filtered and concentrated. The crude was purified by neutral alumina column chromatography using Isolera by eluting with 40% ethyl acetate in pet. ether to afford tert-butyl 4-acetyl-7-(4-bromothiazol-2-yl)-1,4-diazepane-1-carboxylate (10) (0.11 g, 63% pure). LCMS: 404 & 406 (M+1).

### Step (j): Preparation of 1-(5-(4-bromothiazol-2-yl)-1,4-diazepan-1-yl)ethan-1-one (11)

To a solution of tert-butyl 4-acetyl-7-(4-bromothiazol-2-yl)-1,4-diazepane-1-carboxylate (10) (0.11 g, 0.272 mmol) in anhydrous DCM (5 mL) 4N HCl in dioxane (0.14 mL, 0.54 mmol) was added at 0° C. The mixture was stirred at RT for 4 h and then concentrated under reduced pressure to afford 1-(5-(4-bromothiazol-2-yl)-1,4-diazepan-1-yl)ethan-1-one (11) (0.09 g, 75% pure). LCMS: 304 & 306 (M+1).

### Step (k): Preparation of 1-(4-acetyl-7-(4-bromothiazol-2-yl)-1,4-diazepan-1-yl)-2-phenoxyethan-1-one (12)

To an ice-cooled solution of 1-(5-(4-bromothiazol-2-yl)-1,4-diazepan-1-yl)ethan-1-one (11) (0.09 g, 0.264 mmol) in DCM (5 mL) DIPEA (0.13 mL, 0.738 mmol) was added. Phenoxyacetyl chloride (46 mg, 0.27 mmol) was added and the mixture was stirred at RT for 30 min. The reaction was monitored by TLC. The mixture was diluted with DCM (15 mL), washed with water (2×10 mL), brine (10 mL) then dried over anhydrous sodium sulphate, filtered and concentrated. The crude was purified by silica column chromatography using Isolera by eluting with 7% MeOH in DCM to afford 1-(4-acetyl-7-(4-bromothiazol-2-yl)-1,4-diazepan-1-yl)-2-phenoxyethan-1-one (12) (0.09 g, yield: 75%). LCMS: 438 & 440 (M+1).

### Step (l): Preparation of 1-(4-acetyl-7-(4-(pyridin-4-yl)thiazol-2-yl)-1,4-diazepan-1-yl)-2-phenoxyethan-1-one (Example 1)

To a solution of 1-(4-acetyl-7-(4-bromothiazol-2-yl)-1,4-diazepan-1-yl)-2-phenoxyethan-1-one (12) (90 mg, 0.205 mmol) in n-butanol (10 mL) potassium phosphate (87 mg, 0.410 mmol) was added. The mixture was degassed with nitrogen for 10 min. Pyridin-4-ylboronic acid (38 mg, 0.308 mmol), Xphos (10 mg, 0.0205 mmol) and Pd₂(dba)₃ (10 mg, 0.01 mmol) were added and the mixture was degassed with nitrogen for another 10 min then heated to 100° C. for 3 h. The reaction was monitored by TLC and the mixture was filtered through celite and the filtrate was concentrated. The crude was purified by silica column chromatography using Isolera by eluting with 6% MeOH in DCM to afford 1-(4-acetyl-7-(4-(pyridin-4-yl)thiazol-2-yl)-1,4-diazepan-1-yl)-2-phenoxyethan-1-one (Example 1) (0.08 g, yield: 89%). ¹H-NMR (400 MHz, MeOH-d₄): δ 8.64 (d, J=4.40 Hz, 2H), 8.32 (br s, 1H), 7.87 (d, J=4.80 Hz, 2H), 7.28-7.26 (m, 2H), 6.98-6.94 (m, 3H), 6.10-5.68 (m, 1H), 5.02-4.94 (m, 2H), 4.40-4.05 (m, 3H), 3.80-3.60 (m, 2H), 3.40-3.20 (m, 1H), 2.85-2.70 (m, 2H), 2.35-2.10 (m, 3H). LCMS: 437 (M+1).

Examples 2, 4, 5, 7, 10, 12, 17, 23, 26, 27, 40, 98 and 99 (see Table 1—Compounds of formula (I) used in the invention) were synthesized according to the general process used in the invention shown in Scheme 3 and as described below.

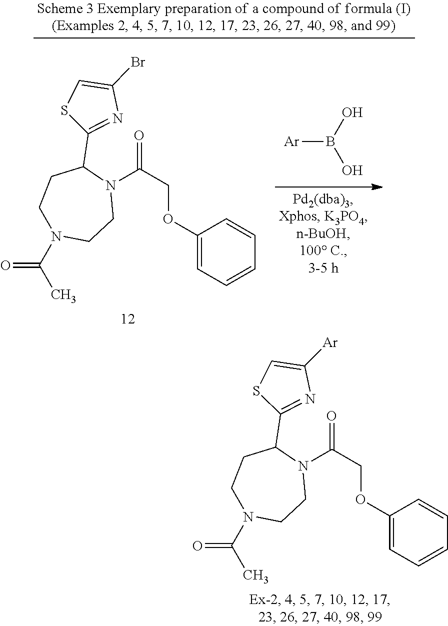

To a stirred solution of bromothiazole compound 12 (1.0 eq) obtained according to step (k) in n-butanol under nitrogen atmosphere were added potassium phosphate tribasic (3.0 eq), the respective aryl boronic acid (1.2 eq), Xphos (10 mol %). The reaction mixture was degassed with nitrogen for 10 min. To this solution, Pd₂(dba)₃ (10 mol %) was added and heated at 100° C. for 3-5 h. The reaction progress was monitored by TLC. Upon completion of the reaction, reaction mixture was diluted with water (20 ml) and extracted with CH₂Cl₂ (50 ml×3). The combined organic extract was dried over anhydrous Na₂SO₄, filtered and concentrated to get crude. The crude was purified by flash column chromatography (silica-gel, 100-200 mesh) using MeOH/CH₂Cl₂ (0-3%) as an eluent to obtain the compounds of Examples 2, 4, 5, 7, 10, 12, 17, 23, 26, 27, 40, 98, and 99. In some cases, both the enantiomers were separated on chiral SFC using LUX-C4 column where the mobile phase used was isopropyl alcohol and liquid carbon dioxide.

Examples 41-44, 50, 56, 72, 74, 77, 100-102 (see Table 1—Compounds of formula (I) used in the invention) were synthesized according to the general process shown in Scheme 4 and as described below.

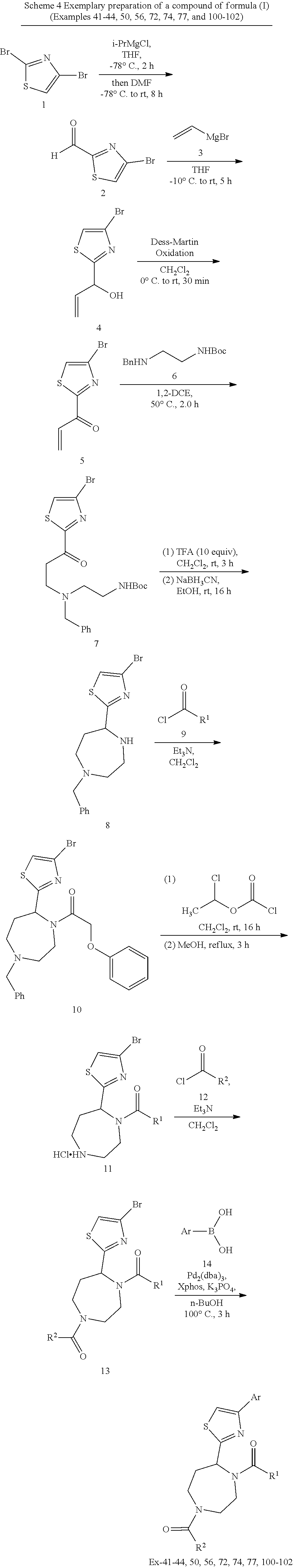

### Step (a): Preparation of 4-bromothiazole-2-carbaldehyde (2)

To solution of 2,4-dibromothiazole (1) (20 g, 82 mmol) in THE (200 ml) was added isopropylmagnesium chloride (49.4 ml, 99 mmol) dropwise at −78° C. and the reaction mixture was stirred for 2 h at the same temperature. To this reaction mixture at −78° C., DMF (37.6 g, 515 mmol) was added dropwise and then the reaction mixture slowly warmed to room temperature and stirred for 8 h. The reaction mixture was quenched with aq. NH₄Cl and extracted in CH₂Cl₂ (500 m L×3). The combined organic extract was washed with water (100 mL), brine solution (100 mL), dried over anhydrous Na₂SO₄, filtered and evaporated under vacuumto get crude. The crude was purified by flash column chromatography (silica-gel, 100-200 mesh) using ethyl acetate/hexanes (0-20%) as an eluent to afford 4-bromothiazole-2-carbaldehyde (2) (9.6 g, 50.0 mmol, 60% yield) as a light-yellow solid. ¹H NMR (400 MHz, CDCl₃): 7.69 (d, J=1.2 Hz, 1H), 9.96 (d, J=1.2 Hz, 1H).

### Step (b): Preparation of 1-(4-bromothiazol-2-yl)prop-2-en-1-ol (4)

Vinylmagnesium bromide (21.87 ml, 1.0 M in THF, 21.87 mmol) was added drop-wise to the stirred solution of 4-bromothiazole-2-carbaldehyde (2) (3.5 g, 18.23 mmol) in THE (30 ml) at −10° C., and the reaction mixture was slowly brought to room temperature and stirred for 5h. The progress of the reaction was monitored by TLC. After completion of the reaction as indicated by TLC, the reaction mixture was quenched with aq. NH₄Cl. The reaction mixture was diluted with EtOAc and layers were separated. The aqueous layer was extracted with EtOAc (50 mL×2). The combined organic extract was washed with water (50 mL), brine solution (50 mL), dried over anhydrous Na₂SO₄, filtered and evaporated under vacuum to get crude. The crude was purified by flash column chromatography (silica-gel, 230-400 mesh) using ethyl acetate/hexanes (0-20%) as an eluent to afford 1-(4-bromothiazol-2-yl)prop-2-en-1-ol (4) (2 g, 9.09 mmol, 50% yield) as a yellow gummy liquid. ¹H-NMR (400 MHz, CDCl₃): δ 7.24 (s, 1H), 6.15-6.13 (m, 1H), 5.51-5.49 (m, 2H), 4.94 (d, J=6.40 Hz, 1H), 3.22 (s, 1H).

### Step (c): Preparation of 1-(4-bromothiazol-2-yl)prop-2-en-1-one (5)

To a stirred solution of 1-(4-bromothiazol-2-yl)prop-2-en-1-ol (4) (2 g, 9.09 mmol) in CH₂Cl₂ (20 ml) was added Dess-Martin Periodinane (4.63 g, 10.90 mmol) at 0° C. under inert atmosphere. The reaction mixture was allowed to warm to room temperature and stirred for 30 min. The progress of the reaction was monitored by TLC. After completion of the reaction, the reaction mixture was quenched with saturated sodium thiosulfate solution. The reaction mixture was diluted with EtOAc and layers were separated. The aqueous layer was extracted again in EtOAc (50 mL×2). The combined organic extract was washed with water (50 mL), brine solution (50 mL), dried over anhydrous Na₂SO₄ and evaporated under vacuum to get crude. The crude was purified by flash column chromatography (silica-gel, 230-400 mesh) using ethyl acetate/hexanes (0-10%) as an eluent to afford 1-(4-bromothiazol-2-yl)prop-2-en-1-one (5) (1.2 g, 5.50 mmol, 60% yield) as an off white solid. ¹H-NMR (400 MHz, CDCl₃): δ 7.63 (s, 1H), 7.52 (dd, J=10.40 Hz, J=17.60 Hz, 1H), 6.76 (dd, J=17.60 Hz, J=1.60 Hz, 1H), 6.07 (dd, J=10.40 Hz, J=1.60 Hz, 1H).

### Step (d): Preparation of tert-butyl (2-(benzyl(3-(4-bromothiazol-2-yl)-3-oxopropyl)amino)ethyl)carbamate (7)

To a stirred solution of 1-(4-bromothiazol-2-yl)prop-2-en-1-one (5) (3.0 g, 13.76 mmol) in 1,2-DCE (3 mL) was added tert-butyl (2-(benzylamino)ethyl)carbamate (3.44 g, 13.76 mmol) and stirred at 50° C. for 2 h. The solvent was evaporated to give tert-butyl (2-(benzyl(3-(4-bromothiazol-2-yl)-3-oxopropyl)amino)ethyl)-carbamate (7) in quantitative yield. ¹H-NMR (400 MHz, CDCl₃): δ 7.57 (s, 1H), 7.40-7.20 (m, 5H), 4.91 (s, 1H), 3.65 (s, 2H), 3.30 (t, J=6.8 Hz, 2H), 3.20 (s, 2H), 3.02 (t, J=6.8, 2H), 2.62-2.59 (m, 2H), 1.45 (s, 9H).

### Step (e): Preparation of 2-(1-benzyl-1,4-diazepan-5-yl)-4-bromothiazole (8)

To a stirred solution of tert-butyl (2-(benzyl(3-(4-bromothiazol-2-yl)-3-oxopropyl)amino)ethyl)carbamate (7) (8 g, 17.08 mmol) in CH₂Cl₂ (80 ml) was added 2,2,2-trifluoroacetic acid (19.60 ml, 256 mmol) and the resulting reaction mixture was stirred at room temperature for 3 h. Then, the reaction mixture was concentrated under reduced pressure to get the residue. To this residue, EtOH (80 ml) was added and stirred for 2 h. To this stirring solution, NaCNBH₃ (4.29 g, 68.3 mmol) was added portion wise and the resulting mixture was stirred at room temperature for 16 h. The solvents were evaporated and the residue was dissolved in CH₂Cl₂ (100 mL). To this solution, 2 M NaOH_{aq} (30 mL) was added and stirred for 20 min. The layers were separated, aqueous layer was extracted with CH₂Cl₂ (50 mL×2), then, the combined organic extract was dried over anhydrous Na₂SO₄, filtered and concentrated under reduced pressure to give the crude. The crude was purified by flash column chromatography (silica-gel, 100-200 mesh) using CH₂Cl₂/MeOH (0-5%) as an eluent to afford 2-(1-benzyl-1,4-diazepan-5-yl)-4-bromothiazole (8) (3.1 g, 8.80 mmol, 51% yield) as an off-white solid. LCMS (ESI, +ve) m/z 352.20 [M+H]+.

### Step (f): Synthesis of Compound 10 of Scheme 4

To a stirred solution of tert-butyl (2-(benzyl(3-(4-bromothiazol-2-yl)-3-oxopropyl)amino)ethyl)carbamate (8) (1.0 eq.) in CH₂Cl₂ was added triethyamine (3.0 eq.) and the reaction mixture cooled to 0° C. To this solution acid chloride 9 (1.2 eq.) was added drop wise. The resulting reaction mixture was allowed to warm to room temperature and stirred for 30 min. The completion of the reaction was monitored by TLC. Upon completion of the reaction, the reaction mixture was diluted with CH₂Cl₂ (50 mL×2), washed with water (20 mL), brine (20 mL) then dried over anhydrous Na₂SO₄, filtered and concentrated to get crude. The crude was purified by flash column chromatography (silica-gel, 100-200 mesh) using CH₂Cl₂/MeOH (0-5%) as an eluent to obtain N-benzyl-homopiperazine derivative (10).

### Step (g): Synthesis of Compound 11 of Scheme 4

To a stirred solution of N-benzyl-homopiperazine derivative (10) (1.0 eq.) in CH₂Cl₂ was added 1-chloroethyl carbonochloridate (3.0 eq.) at room temperature and stirred for 16 h. After this time, volatiles were removed under rotary evaporator and to this crude material was added MeOH and refluxed for 3 h. Solvents were evaporated and the residue was re-dissolved in CH₂Cl₂ and evaporated. Again added 5-10 mL of CH₂Cl₂ and sonicated for 5 min, at this point, a white precipitate was formed, which was filtered, washed with CH₂Cl₂ to obtain crude of homopiperazine hydrochloride derivative 11 as a solid, which was as such used in the next step without further purification.

### Step (h): Synthesis of Compound 13 of Scheme 4

To a stirred solution of homopiperazine hydrochloride derivative 11 (1.0 eq.) in CH₂Cl₂ was added triethylamine (5.0 eq.) and the reaction mixture was cooled to 0° C. To this reaction mixture, the respective acetyl chloride 12 (1.2 eq.) was added and the resulting reaction mixture was allowed to warm to room temperature and stirred for 1 h. After completion of the reaction, reaction mixture was diluted with water (10 ml) and extracted with CH₂Cl₂ (20 ml×3). The combined organic layer was dried over anhydrous Na₂SO₄, filtered and concentrated to get crude product. The crude was purified by flash column chromatography (silica-gel, 100-200 mesh) using MeOH/CH₂Cl₂ (0-3%) as an eluent to afford compound 13.

### Step (i): Synthesis of a Compound Used in the Invention, i.e. of Examples 41-44, 50, 56, 72, 74, 77, and 100-102

To a stirred solution of compound 13 (1.0 eq) in n-butanol under nitrogen atmosphere were added potassium phosphate tribasic (3.0 eq), the respective aryl boronic acid (14) (1.2 eq), Xphos (10 mol %). The reaction mixture was degassed with nitrogen for 10 min. To this solution, Pd₂(dba)₃ (10 mol %) was added and heated at 100° C. for 3-5 h. The reaction progress was monitored by TLC. Upon completion of the reaction, reaction mixture was diluted with water (20 ml) and extracted with CH₂Cl₂ (50 ml×3). The combined organic extract was dried over anhydrous Na₂SO₄, filtered and concentrated to get crude. The crude was purified by flash column chromatography (silica-gel, 100-200 mesh) using MeOH/CH₂Cl₂ (0-3%) as an eluent to provide corresponding products.

### 3.2 Examples 2 to 102

The compounds exemplifying the invention are described in Table 1.

The compounds described in Table 1 can be obtained according to the processes described in Schemes 1, 2, 3 and 4.

TABLE 1
Compounds of formula (I) used in the invention
Note: ¹H NMR data indicates these compounds exists as rotamers due to the presence of
two tertiary amides. In some cases, enantiomers are separated but absolute
stereochemistry for each enantiomer is not yet assigned.

| Ex | Structure | ¹H-NMR | MS |
|---|---|---|---|
| 1 | 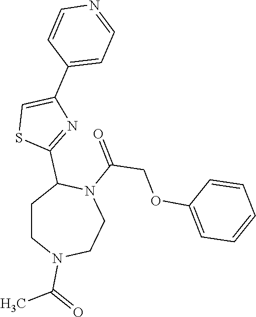 [SMILES: CC(=O)N1CCC(c2nc(-c3ccncc3)cs2)N(C(=O)COc2ccccc2)CC1] | ¹H NMR (400 MHz, MeOH-d₄) δ 2.09 (s, 0.80H), 2.14 (s, 0.52H), 2.16 (s, 0.65H), 2.20 (s, 0.95H), 2.41-2.83 (m, 2H), 3.12-3.27 (m, 0.5H), 3.48-3.77 (m, 2.5H), 3.92-4.43 (m, 3H), 4.93-5.06 (m, 2H), 5.70-5.84 (m, 0.5H), 5.94-6.07 (m, 0.5H), 6.87-7.05 (m, 3H), 7.21-7.34 (m, 2H), 7.95- 8.00 (m, 2H), 8.19 (s, 0.23H), 8.20 (s, 0.30H), 8.27 (s, 0.15H), 8.27 (s, 0.25H), 8.56-8.63 (m, 2H). Enantiomer 1: SFC-Chiral purity = 99.3%, RT = 7.91 min; Enantiomer 2: SFC-Chiral purity = 99.3%, RT = 10.52 min | 437.2 (M + 1) |
| 2 | 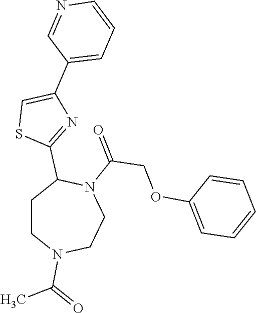 [SMILES: CC(=O)N1CCC(c2nc(-c3cccnc3)cs2)N(C(=O)COc2ccccc2)CC1] | ¹H NMR (400 MHz, MeOH-d₄) δ 2.08 (s, 0.85H), 2.13 (s, 0.56H), 2.14 (s, 0.60H), 2.18 (s, 0.97H), 2.41-2.76 (m, 2H), 3.12-3.28 (m, 0.5H), 3.45-3.79 (m, 2.5H), 3.87-4.41 (m, 3H), 4.90-5.06 (m, 2H), 5.67-5.79 (m, 0.5H), 5.90-6.06 (m, 0.5H), 6.89-7.03 (m, 3H), 7.19-7.31 (m, 2H), 7.50 (dd, J = 8.1, 5.0 Hz, 1H), 7.99 (s, 0.20H), 8.00 (s, 0.30H), 8.07 (s, 0.22H), 8.08 (s, 0.27H), 8.33- 8.39 (m, 1H), 8.48-8.54 (m, 1H), 9.09-9.15 (m, 1H). Enantiomer 1: SFC-Chiral purity = >99.9%, RT = 5.51 min; Enantiomer 2: SFC-Chiral purity = >99.9%, RT = 8.65 min | 437.2 (M + 1) |
| 3 | 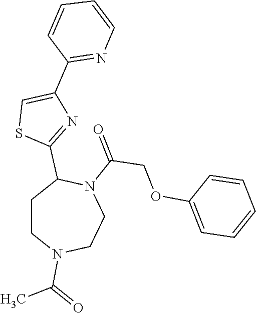 [SMILES: CC(=O)N1CCC(c2nc(-c3ccccn3)cs2)N(C(=O)COc2ccccc2)CC1] | | |
| 4 | 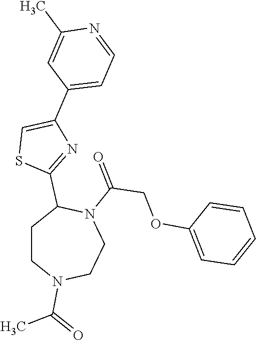 [SMILES: CC(=O)N1CCC(c2nc(-c3ccnc(C)c3)cs2)N(C(=O)COc2ccccc2)CC1] | ¹H NMR (400 MHz, MeOH-d₄) δ 2.10 (s, 0.86H), 2.15 (s, 0.56H), 2.16 (s, 0.65H), 2.20 (s, 0.95H), 2.42-2.79 (m, 5H), 3.13-3.26 (m, 0.5H), 3.49-3.79 (m, 2.5H), 3.90-4.42 (m, 3H), 4.91-5.06 (m, 2H), 5.71-5.81 (m, 0.5H), 5.93-6.06 (m, 0.5H), 6.90-7.04 (m, 3H), 7.21-7.34 (m, 2H), 7.77 (dd, J = 5.4, 1.6 Hz, 1H), 7.86 (d, J = 6.8 Hz, 1H), 8.14-8.25 (m, 1H), 8.43-8.47 (m, 1H). Enantiomer 1: SFC-Chiral purity = >99.9%, RT = 5.06 min; Enantiomer 2: SFC-Chiral purity = >99.9%, RT = 7.95 min | 451.2 (M + 1) |
| 5 | 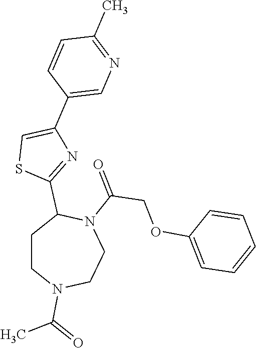 [SMILES: CC(=O)N1CCC(c2nc(-c3ccc(C)nc3)cs2)N(C(=O)COc2ccccc2)CC1] | ¹H NMR (400 MHz, MeOH-d₄) δ 2.09 (s, 0.90H), 2.14 (s, 0.56H), 2.16 (s, 0.66H), 2.19 (s, 0.97H), 2.41-2.76 (m, 5H), 3.13-3.27 (m, 0.5H), 3.49-3.79 (m, 2.5H), 3.89-4.42 (m, 3H), 4.93-5.05 (m, 2H), 5.70-5.79 (m, 0.5H), 5.95-6.05 (m, 0.5H), 6.91-7.04 (m, 3H), 7.21-7.33 (m, 2H), 7.39 (d, J = 8.0 Hz, 1H), 7.92 (s, 0.21H), 7.93 (s, 0.30H), 8.01 (s, 0.18H), 8.01 (s, 0.25H), 8.25 (dd, J = 8.0, 2.0 Hz, 1H), 8.97-9.01 (m, 1H). Enantiomer 1: SFC-Chiral purity = >99.9%, RT = 4.61 min; Enantiomer 2: SFC-Chiral purity = >99.9%, RT = 7.53 min | 451.1 (M + 1) |
| 6 | 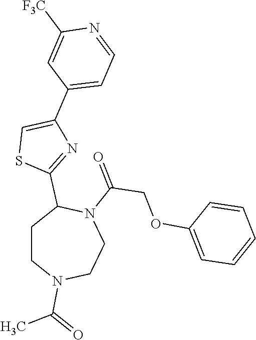 [SMILES: CC(=O)N1CCC(c2nc(-c3ccnc(C(F)(F)F)c3)cs2)N(C(=O)COc2ccccc2)CC1] | | |
| 7 | 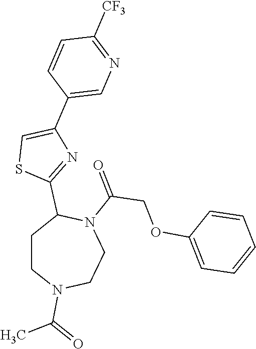 [SMILES: CC(=O)N1CCC(c2nc(-c3ccc(C(F)(F)F)nc3)cs2)N(C(=O)COc2ccccc2)CC1] | ¹H NMR (400 MHz, MeOH-d₄) δ 2.09 (s, 0.85H), 2.14 (s, 0.53H), 2.16 (s, 0.68H), 2.20 (s, 0.97H), 2.43-2.79 (m, 2H), 3.13-3.31 (m, 0.5H), 3.48-3.79 (m, 2.5H), 3.91-4.43 (m, 3H), 4.93-5.06 (m, 2H), 5.73-5.83 (m, 0.5H), 5.96-6.07 (m, 0.5H), 6.89-7.06 (m, 3H), 7.20-7.35 (m, 2H), 7.89 (dd, J = 8.2, 0.8 Hz, 1H), 8.17 (s, 0.22H), 8.18 (s, 0.30H), 8.25 (s, 0.16H), 8.26 (s, 0.25H), 8.51- 8.59 (m, 1H), 9.25-9.32 (m, 1H). | 505.2 (M + 1) |
| 8 | 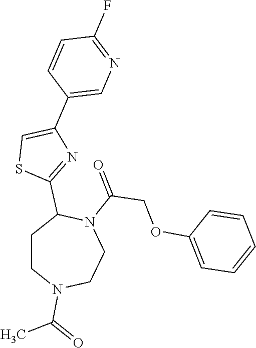 [SMILES: CC(=O)N1CCC(c2nc(-c3ccc(F)nc3)cs2)N(C(=O)COc2ccccc2)CC1] | | |
| 9 | 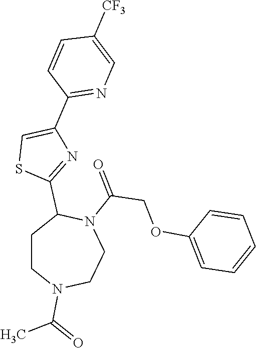 [SMILES: CC(=O)N1CCC(c2nc(-c3ccc(C(F)(F)F)cn3)cs2)N(C(=O)COc2ccccc2)CC1] | | |
| 10 | 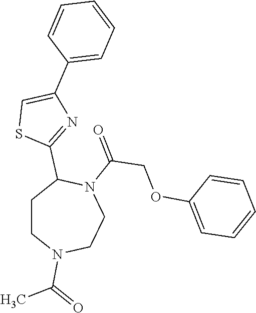 [SMILES: CC(=O)N1CCC(c2nc(-c3ccccc3)cs2)N(C(=O)COc2ccccc2)CC1] | ¹H NMR (400 MHz, MeOH-d₄) δ 2.10 (s, 0.94H), 2.14 (s, 0.56H), 2.15 (s, 0.55H), 2.19 (s, 0.95H), 2.43-2.78 (m, 2H), 3.13-3.30 (m, 0.5H), 3.49-3.77 (m, 2.5H), 3.89-4.42 (m, 3H), 4.93-5.07 (m, 2H), 5.66-5.76 (m, 0.5H), 5.96-6.10 (m, 0.5H), 6.92-7.05 (m, 3H), 7.22-7.38 (m, 3H), 7.39-7.47 (m, 2H), 7.76 (s, 0.21H), 7.77 (s, 0.28H), 7.85 (s, 0.19H), 7.86 (s, 0.25H), 7.91- 7.98 (m, 2H). | 436.2 (M + 1) |
| 11 | 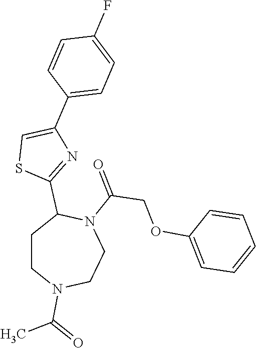 [SMILES: CC(=O)N1CCC(c2nc(-c3ccc(F)cc3)cs2)N(C(=O)COc2ccccc2)CC1] | | |
| 12 | 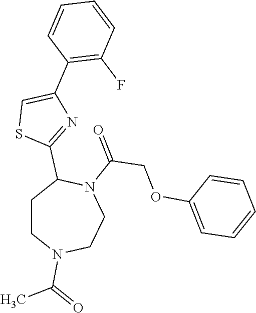 [SMILES: CC(=O)N1CCC(c2nc(-c3ccccc3F)cs2)N(C(=O)COc2ccccc2)CC1] | ¹H NMR (400 MHz, MeOH-d₄) δ 2.09 (s, 0.93H), 2.14 (s, 0.59H), 2.15 (s, 0.64H), 2.19 (s, 0.96H), 2.45-2.78 (m, 2H), 3.12-3.27 (m, 0.5H), 3.49-3.76 (m, 2.5H), 3.96-4.44 (m, 3H), 4.93-5.07 (m, 2H), 5.69-5.79 (m, 0.5H), 5.99-6.10 (m, 0.5H), 6.91-7.06 (m, 3H), 7.18-7.42 (m, 5H), 7.81-7.94 (m, 1H), 8.13-8.22 (m, 1H). Enantiomer 1: SFC-Chiral purity = >99.9%, RT = 3.55 min; Enantiomer 2: SFC-Chiral purity = >99.9%, RT = 6.71 min | 454.2 (M + 1) |
| 13 | 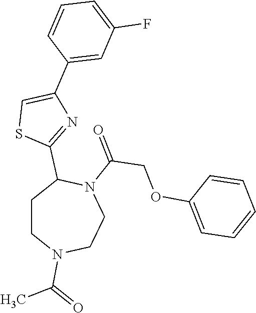 [SMILES: CC(=O)N1CCC(c2nc(-c3cccc(F)c3)cs2)N(C(=O)COc2ccccc2)CC1] | | |
| 14 | 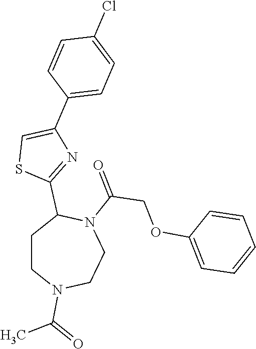 [SMILES: CC(=O)N1CCC(c2nc(-c3ccc(Cl)cc3)cs2)N(C(=O)COc2ccccc2)CC1] | | |
| 15 | 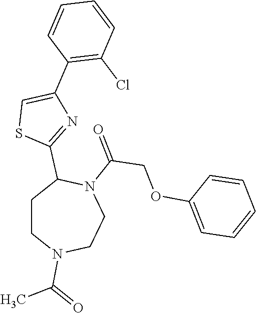 [SMILES: CC(=O)N1CCC(c2nc(-c3ccccc3Cl)cs2)N(C(=O)COc2ccccc2)CC1] | | |
| 16 | 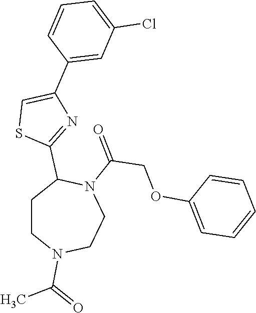 [SMILES: CC(=O)N1CCC(c2nc(-c3cccc(Cl)c3)cs2)N(C(=O)COc2ccccc2)CC1] | | |
| 17 | 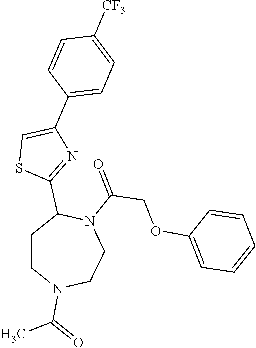 [SMILES: CC(=O)N1CCC(c2nc(-c3ccc(C(F)(F)F)cc3)cs2)N(C(=O)COc2ccccc2)CC1] | ¹H NMR (400 MHz, MeOH-d₄) δ 2.09 (s, 0.84H), 2.14 (s, 0.56H), 2.16 (s, 0.66H), 2.19 (s, 0.90H), 2.45-2.78 (m, 2H), 3.13-3.27 (m, 0.5H), 3.49-3.77 (m, 2.5H), 3.91-4.42 (m, 3H), 4.93-5.06 (m, 2H), 5.70-5.80 (m, 0.5H), 5.97-6.08 (m, 0.5H), 6.91-7.04 (m, 3H), 7.21-7.33 (m, 2H), 7.74 (d, J = 8.4 Hz, 2H), 7.98 (s, 0.21H), 7.99 (s, 0.30H), 8.07 (s, 0.17H), 8.07 (s, 0.26H), 8.15 (d, J = 8.0 Hz, 2H). | 504.2 (M + 1) |
| 18 | 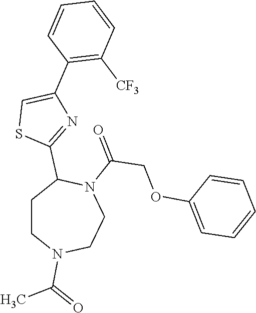 [SMILES: CC(=O)N1CCC(c2nc(-c3ccccc3C(F)(F)F)cs2)N(C(=O)COc2ccccc2)CC1] | | |
| 19 | 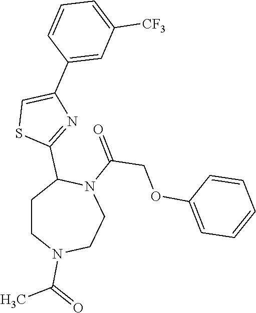 [SMILES: CC(=O)N1CCC(c2nc(-c3cccc(C(F)(F)F)c3)cs2)N(C(=O)COc2ccccc2)CC1] | | |
| 20 | 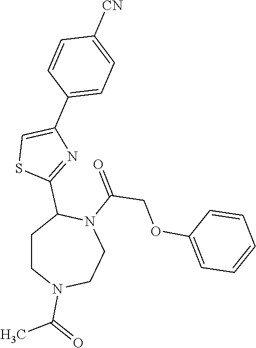 [SMILES: CC(=O)N1CCC(c2nc(-c3ccc(C#N)cc3)cs2)N(C(=O)COc2ccccc2)CC1] | | |
| 21 | 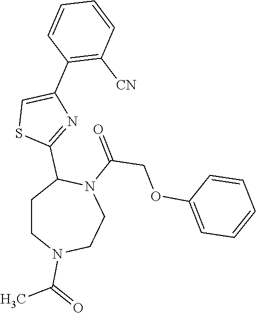 [SMILES: CC(=O)N1CCC(c2nc(-c3ccccc3C#N)cs2)N(C(=O)COc2ccccc2)CC1] | | |
| 22 | 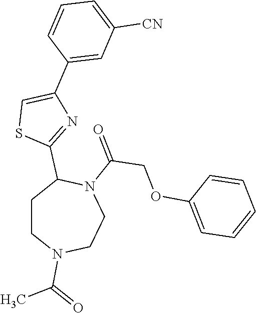 [SMILES: CC(=O)N1CCC(c2nc(-c3cccc(C#N)c3)cs2)N(C(=O)COc2ccccc2)CC1] | | |
| 23 | 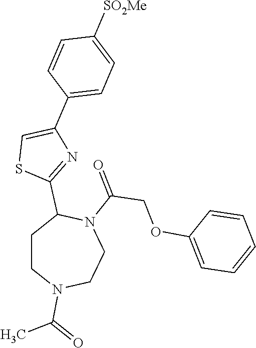 [SMILES: CC(=O)N1CCC(c2nc(-c3ccc(S(C)(=O)=O)cc3)cs2)N(C(=O)COc2ccccc2)CC1] | ¹H NMR (400 MHz, MeOH-d₄) δ 2.09 (s, 0.82H), 2.15 (s, 0.54H), 2.16 (s, 0.60H), 2.19 (s, 0.91H), 2.48-2.79 (m, 2H), 3.17 (s, 3H), 3.20-3.31 (m, 0.5H), 3.47-3.81 (m, 2.5H), 3.89-4.43 (m, 3H), 4.92-5.07 (m, 2H), 5.72-5.81 (m, 0.5H), 5.96-6.08 (m, 0.5H), 6.89-7.06 (m, 3H), 7.20-7.35 (m, 2H), 7.98-8.06 (m, 2H), 8.06 (s, 0.20H), 8.08 (s, 0.29H), 8.15 (s, 0.15H), 8.15 (s, 0.25H), 8.18- 8.26 (m, 2H). | 513.9 (M + 1) |
| 24 | 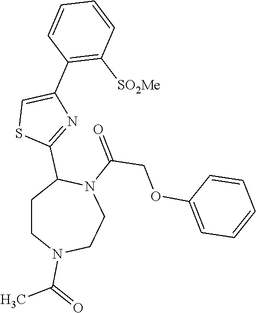 [SMILES: CC(=O)N1CCC(c2nc(-c3ccccc3S(C)(=O)=O)cs2)N(C(=O)COc2ccccc2)CC1] | | |
| 25 | 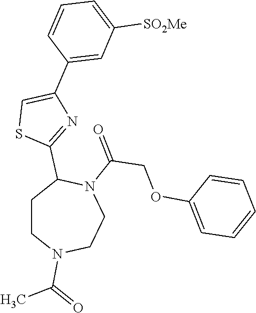 [SMILES: CC(=O)N1CCC(c2nc(-c3cccc(S(C)(=O)=O)c3)cs2)N(C(=O)COc2ccccc2)CC1] | | |
| 26 | 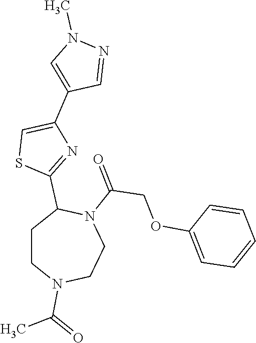 [SMILES: CC(=O)N1CCC(c2nc(-c3cnn(C)c3)cs2)N(C(=O)COc2ccccc2)CC1] | ¹H NMR (400 MHz, MeOH-d₄) δ 2.09 (s, 0.90H), 2.14 (s, 0.59H), 2.15 (s, 0.61H), 2.18 (s, 0.91H), 2.38-2.76 (m, 2H), 3.15-3.29 (m, 0.5H), 3.45-3.77 (m, 2.5H), 3.94 (s, 3H), 3.94-4.41 (m, 3H), 4.91-5.06 (m, 2H), 5.62-5.72 (m, 0.5H), 5.91-6.04 (m, 0.5H), 6.89-7.05 (m, 3H), 7.21-7.35 (m, 2H), 7.45 (s, 0.20H), 7.46 (s, 0.29H), 7.53 (s, 0.18H), 7.54 (s, 0.27H), 7.84-7.90 (m, 1H), 7.97- 8.03 (m, 1H). | 440.2 (M + 1) |
| 27 | 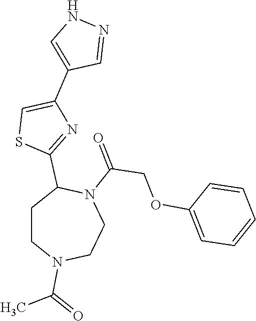 [SMILES: CC(=O)N1CCC(c2nc(-c3cn[nH]c3)cs2)N(C(=O)COc2ccccc2)CC1] | ¹H NMR (400 MHz, MeOH-d₄) δ 2.09 (s, 0.82H), 2.14 (s, 0.51H), 2.15 (s, 0.73H), 2.19 (s, 1.02H), 2.41-2.78 (m, 2H), 3.13-3.29 (m, 0.5H), 3.47-3.76 (m, 2.5H), 3.92-4.41 (m, 3H), 4.93-5.04 (m, 2H), 5.68-5.77 (m, 0.5H), 5.91-5.98 (m, 0.5H), 6.49-6.53 (m, 1H), 6.90-7.04 (m, 3H), 7.22-7.33 (m, 2H), 7.46 (s, 0.22H), 7.47 (s, 0.31H), 7.54 (s, 0.19H), 7.55 (s, 0.23H), 7.71- 7.76 (m, 1H), 8.28-8.36 (m, 1H). | 426.2 (M + 1) |
| 28 | 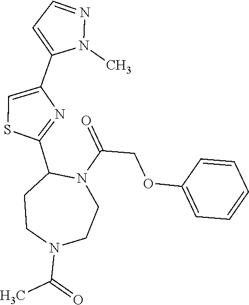 [SMILES: CC(=O)N1CCC(c2nc(-c3ccnn3C)cs2)N(C(=O)COc2ccccc2)CC1] | | |
| 29 | 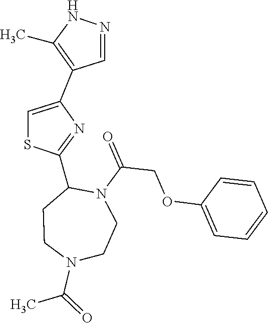 [SMILES: CC(=O)N1CCC(c2nc(-c3cn[nH]c3C)cs2)N(C(=O)COc2ccccc2)CC1] | | |
| 30 | 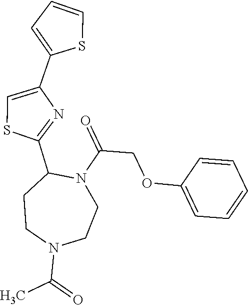 [SMILES: CC(=O)N1CCC(c2nc(-c3cccs3)cs2)N(C(=O)COc2ccccc2)CC1] | | |
| 31 | 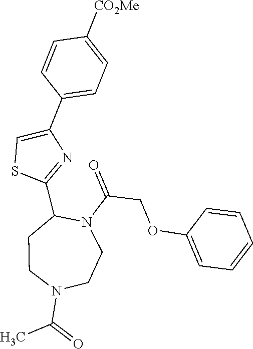 [SMILES: COC(=O)c1ccc(-c2csc(C3CCN(C(C)=O)CCN3C(=O)COc3ccccc3)n2)cc1] | | |
| 32 | 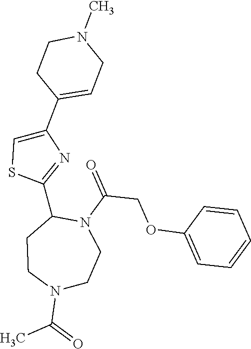 [SMILES: CC(=O)N1CCC(c2nc(C3=CCN(C)CC3)cs2)N(C(=O)COc2ccccc2)CC1] | | |
| 33 | 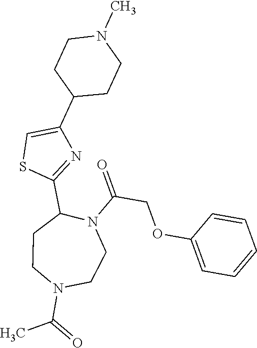 [SMILES: CC(=O)N1CCC(c2nc(C3CCN(C)CC3)cs2)N(C(=O)COc2ccccc2)CC1] | | |
| 34 | 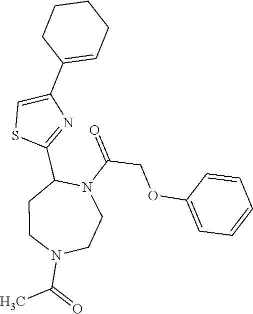 [SMILES: CC(=O)N1CCC(c2nc(C3=CCCCC3)cs2)N(C(=O)COc2ccccc2)CC1] | | |
| 35 | 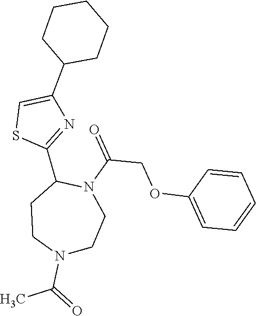 [SMILES: CC(=O)N1CCC(c2nc(C3CCCCC3)cs2)N(C(=O)COc2ccccc2)CC1] | | |
| 36 | 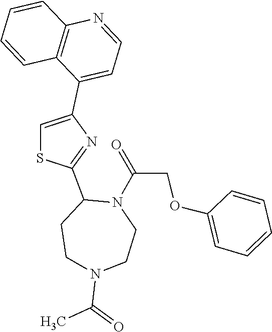 [SMILES: CC(=O)N1CCC(c2nc(-c3ccnc4ccccc34)cs2)N(C(=O)COc2ccccc2)CC1] | | |
| 37 | 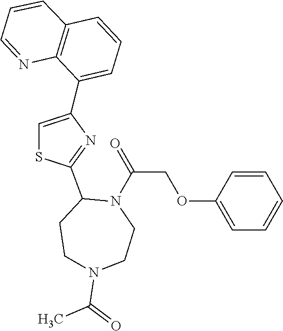 [SMILES: CC(=O)N1CCC(c2nc(-c3cccc4cccnc34)cs2)N(C(=O)COc2ccccc2)CC1] | | |
| 38 | 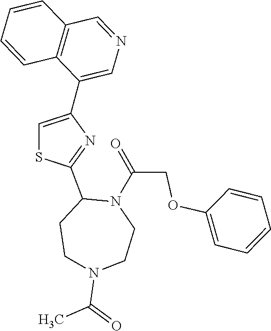 [SMILES: CC(=O)N1CCC(c2nc(-c3cncc4ccccc34)cs2)N(C(=O)COc2ccccc2)CC1] | | |
| 39 | 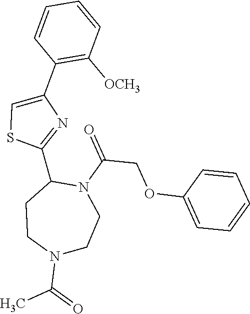 [SMILES: COc1ccccc1-c1csc(C2CCN(C(C)=O)CCN2C(=O)COc2ccccc2)n1] | | |
| 40 | 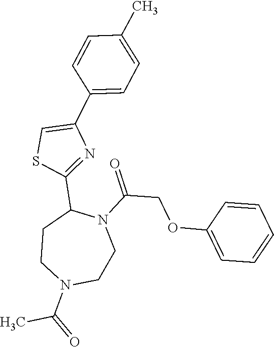 [SMILES: CC(=O)N1CCC(c2nc(-c3ccc(C)cc3)cs2)N(C(=O)COc2ccccc2)CC1] | ¹H NMR (400 MHz, MeOH-d₄) δ 2.10 (s, 0.95H), 2.14 (s, 0.62H), 2.15 (s, 0.56H), 2.19 (s, 0.91H), 2.39 (s, 3H), 2.43-2.77 (m, 2H), 3.14-3.26 (m, 0.5H), 3.48-3.77 (m, 2.5H), 3.88-4.43 (m, 3H), 4.93-5.07 (m, 2H), 5.64-5.76 (m, 0.5H), 5.96-6.09 (m, 0.5H), 6.91-7.05 (m, 3H), 7.22-7.33 (m, 4H), 7.68 (s, 0.18H), 7.70 (s, 0.27H), 7.77 (s, 0.23H), 7.78 (s, 0.26H), 7.79-7.86 (m, 2H). | 450.3 (M + 1) |
| 41 | 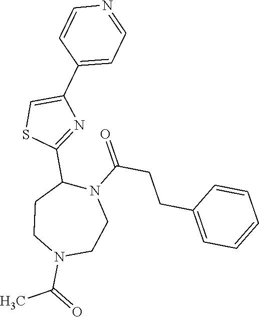 [SMILES: CC(=O)N1CCC(c2nc(-c3ccncc3)cs2)N(C(=O)CCc2ccccc2)CC1] | ¹H NMR (400 MHz, MeOH-d₄) δ 2.09 (s, 0.79H), 2.10 (s, 0.69H), 2.15 (s, 0.46H), 2.17 (s, 0.99H), 2.32-2.64 (m, 2H), 2.74-2.93 (m, 2H), 2.95-3.21 (m, 4H), 3.39-3.59 (m, 1H), 3.69-4.39 (m, 3H), 5.58 (q, J = 6.8 Hz, 0.5H), 5.95-6.06 (m, 0.5H), 7.13-7.21 (m, 1H), 7.21-7.29 (m, 4H), 7.93-8.01 (m, 2H), 8.18 (s, 0.26H), 8.19 (s, 0.31H), 8.22 (s, 0.15H), 8.23 (s, 0.21H), 8.55- 8.63 (m, 2H). | 435.2 (M + 1) |
| 42 | 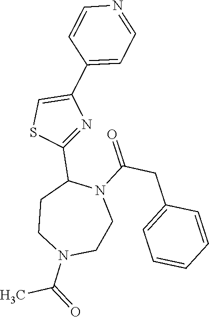 [SMILES: CC(=O)N1CCC(c2nc(-c3ccncc3)cs2)N(C(=O)Cc2ccccc2)CC1] | ¹H NMR (400 MHz, MeOH-d₄) δ 2.07 (s, 0.82H), 2.08 (s, 0.54H), 2.10 (s, 0.74H), 2.18 (s, 0.93H), 2.21-2.68 (m, 2H), 3.03-3.20 (m, 0.5H), 3.35-3.68 (m, 2.5H), 3.76-4.40 (m, 5H), 5.69 (t, J = 6.4 Hz, 0.5H), 5.99-6.09 (m, 0.5H), 7.24-7.31 (m, 1H), 7.31- 7.37 (m, 4H), 7.93-8.00 (m, 2H), 8.18 (s, 0.25H), 8.18 (s, 0.28H), 8.22 (s, 0.23H), 8.23 (s, 0.17H), 8.55-8.63 (m, 2H). | 421.2 (M + 1) |
| 43 | 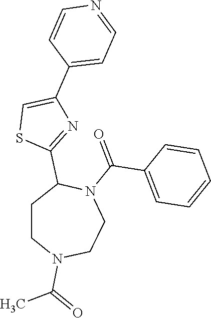 [SMILES: CC(=O)N1CCC(c2nc(-c3ccncc3)cs2)N(C(=O)c2ccccc2)CC1] | ¹H NMR (400 MHz, MeOH-d₄) δ 2.06 (s, 0.64H), 2.20 (s, 2.36H), 2.38-2.87 (m, 2H), 3.46-4.10 (m, 5H), 4.15-4.61 (m, 1H), 5.30-5.42 (m, 0.5H), 6.04-6.19 (m, 0.5H), 7.50 (s, 5H), 8.01 (s, 2H), 8.23-8.32 (m, 1H), 8.57- 8.64 (m, 2H). Enantiomer 1: SFC-Chiral purity = 99.1%, RT = 7.90 min; Enantiomer 2: SFC-Chiral purity = 98.9%, RT = 11.95 min | 407.2 (M + 1) |
| 44 | 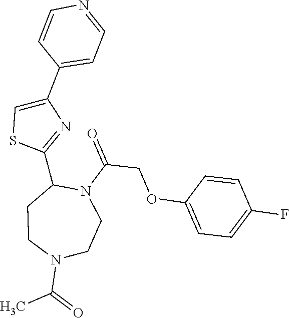 [SMILES: CC(=O)N1CCC(c2nc(-c3ccncc3)cs2)N(C(=O)COc2ccc(F)cc2)CC1] | ¹H NMR (400 MHz, MeOH-d₄) δ 2.10 (s, 0.81H), 2.16 (s, 0.45H), 2.17 (s, 0.64H), 2.19 (s, 0.96H), 2.43-2.80 (m, 2H), 3.13-3.29 (m, 0.5H), 3.50-3.78 (m, 2.5H), 3.92-4.44 (m, 3H), 4.91-5.04 (m, 2H), 5.68-5.78 (m, 0.5H), 5.94-6.06 (m, 0.5H), 6.83-7.07 (m, 4H), 7.93-8.01 (m, 2H), 8.19 (s, 0.21H), 8.20 (s, 0.30H), 8.27 (s, 0.19H), 8.27 (s, 0.21H), 8.52- 8.64 (m, 2H). | 455.2 (M + 1) |
| 45 | 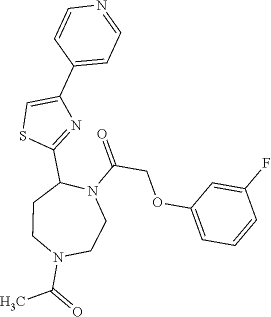 [SMILES: CC(=O)N1CCC(c2nc(-c3ccncc3)cs2)N(C(=O)COc2cccc(F)c2)CC1] | | |
| 46 | 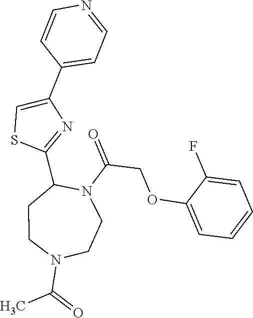 [SMILES: CC(=O)N1CCC(c2nc(-c3ccncc3)cs2)N(C(=O)COc2ccccc2F)CC1] | | |
| 47 | 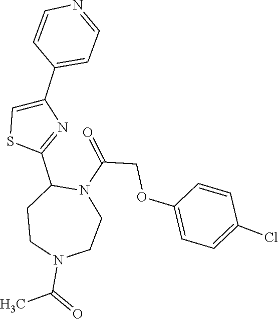 [SMILES: CC(=O)N1CCC(c2nc(-c3ccncc3)cs2)N(C(=O)COc2ccc(Cl)cc2)CC1] | | |
| 48 | 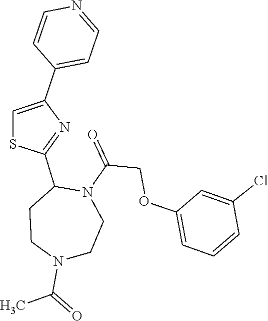 [SMILES: CC(=O)N1CCC(c2nc(-c3ccncc3)cs2)N(C(=O)COc2cccc(Cl)c2)CC1] | | |
| 49 | 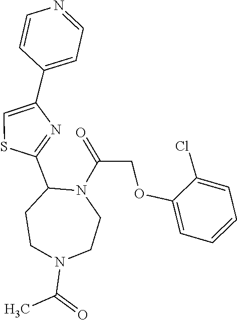 [SMILES: CC(=O)N1CCC(c2nc(-c3ccncc3)cs2)N(C(=O)COc2ccccc2Cl)CC1] | | |
| 50 | 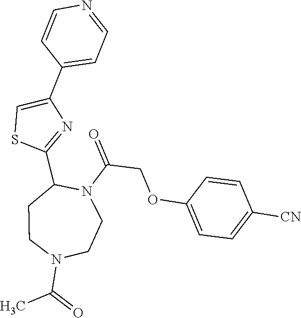 [SMILES: CC(=O)N1CCC(c2nc(-c3ccncc3)cs2)N(C(=O)COc2ccc(C#N)cc2)CC1] | ¹H NMR (400 MHz, MeOH-d₄) δ 2.10 (s, 0.77H), 2.17 (s, 0.47H), 2.18 (s, 0.63H), 2.20 (s, 1.03H), 2.42-2.83 (m, 2H), 3.14-3.28 (m, 0.5H), 3.47-3.83 (m, 2.5H), 3.92-4.44 (m, 3H), 5.03-5.21 (m, 2H), 5.65-5.74 (m, 0.5H), 5.92-6.04 (m, 0.5H), 7.03-7.19 (m, 2H), 7.58-7.71 (m, 2H), 7.94-8.02 (m, 2H), 8.19 (s, 0.20H), 8.21 (s, 0.33H), 8.28 (s, 0.16H), 8.28 (s, 0.19H), 8.58- 8.65 (m, 2H). | 462.2 (M + 1) |
| 51 | 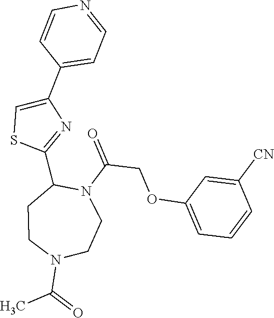 [SMILES: CC(=O)N1CCC(c2nc(-c3ccncc3)cs2)N(C(=O)COc2cccc(C#N)c2)CC1] | | |
| 52 | 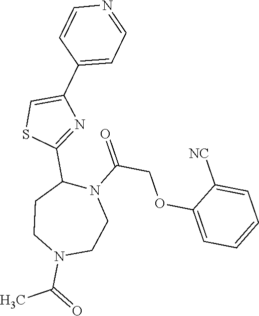 [SMILES: CC(=O)N1CCC(c2nc(-c3ccncc3)cs2)N(C(=O)COc2ccccc2C#N)CC1] | | |
| 53 | 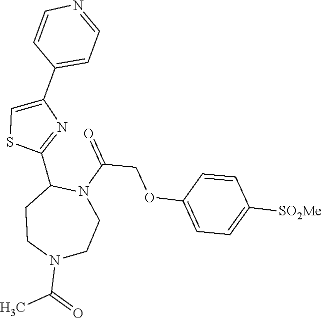 [SMILES: CC(=O)N1CCC(c2nc(-c3ccncc3)cs2)N(C(=O)COc2ccc(S(C)(=O)=O)cc2)CC1] | | |
| 54 | 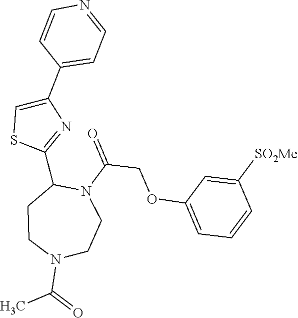 [SMILES: CC(=O)N1CCC(c2nc(-c3ccncc3)cs2)N(C(=O)COc2cccc(S(C)(=O)=O)c2)CC1] | | |
| 55 | 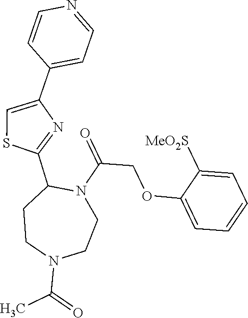 [SMILES: CC(=O)N1CCC(c2nc(-c3ccncc3)cs2)N(C(=O)COc2ccccc2S(C)(=O)=O)CC1] | | |
| 56 | 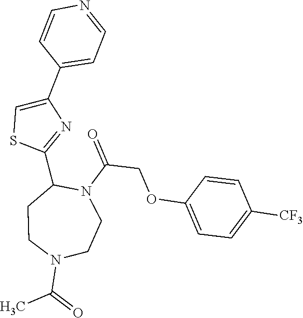 [SMILES: CC(=O)N1CCC(c2nc(-c3ccncc3)cs2)N(C(=O)COc2ccc(C(F)(F)F)cc2)CC1] | ¹H NMR (400 MHz, MeOH-d₄) δ 2.10 (s, 0.77H), 2.17 (s, 0.48H), 2.18 (s, 0.63H), 2.20 (s, 0.99H), 2.42-2.84 (m, 2H), 3.14-3.30 (m, 0.5H), 3.48-3.78 (m, 2.5H), 3.94-4.44 (m, 3H), 4.98-5.17 (m, 2H), 5.69-5.77 (m, 0.5H), 5.94-6.04 (m, 0.5H), 7.03-7.19 (m, 2H), 7.50-7.63 (m, 2H), 7.94-8.01 (m, 2H), 8.19 (s, 0.21H), 8.21 (s, 0.33H), 8.28 (s, 0.15H), 8.28 (s, 0.23H), 8.56- 8.62 (m, 2H). | 505.2 (M + 1) |
| 57 | 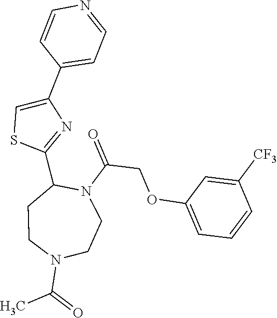 [SMILES: CC(=O)N1CCC(c2nc(-c3ccncc3)cs2)N(C(=O)COc2cccc(C(F)(F)F)c2)CC1] | | |
| 58 | 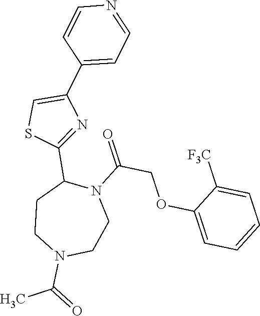 [SMILES: CC(=O)N1CCC(c2nc(-c3ccncc3)cs2)N(C(=O)COc2ccccc2C(F)(F)F)CC1] | | |
| 59 | 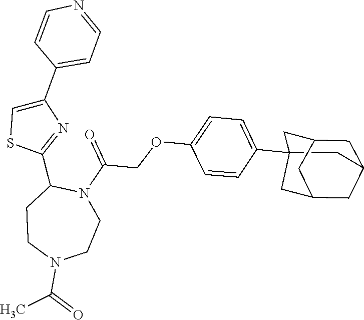 [SMILES: CC(=O)N1CCC(c2nc(-c3ccncc3)cs2)N(C(=O)COc2ccc(C34CC5CC(CC(C5)C3)C4)cc2)CC1] | | |
| 60 | 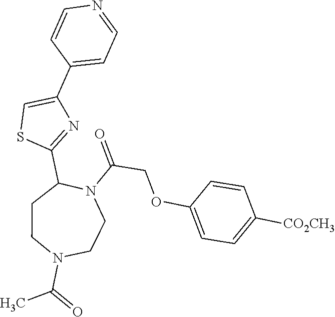 [SMILES: COC(=O)c1ccc(OCC(=O)N2CCN(C(C)=O)CCC2c2nc(-c3ccncc3)cs2)cc1] | | |
| 61 | 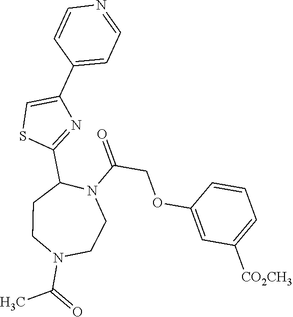 [SMILES: COC(=O)c1cccc(OCC(=O)N2CCN(C(C)=O)CCC2c2nc(-c3ccncc3)cs2)c1] | | |
| 62 | 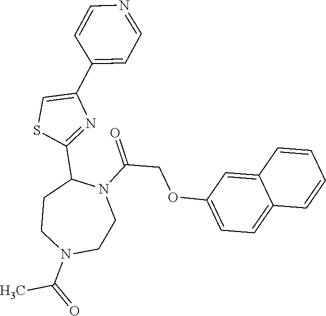 [SMILES: CC(=O)N1CCC(c2nc(-c3ccncc3)cs2)N(C(=O)COc2ccc3ccccc3c2)CC1] | | |
| 63 | 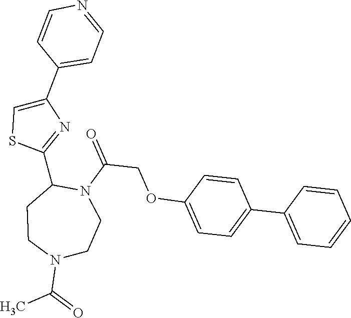 [SMILES: CC(=O)N1CCC(c2nc(-c3ccncc3)cs2)N(C(=O)COc2ccc(-c3ccccc3)cc2)CC1] | | |
| 64 | 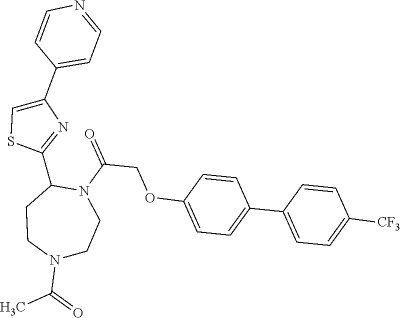 [SMILES: CC(=O)N1CCC(c2nc(-c3ccncc3)cs2)N(C(=O)COc2ccc(-c3ccc(C(F)(F)F)cc3)cc2)CC1] | | |
| 65 | 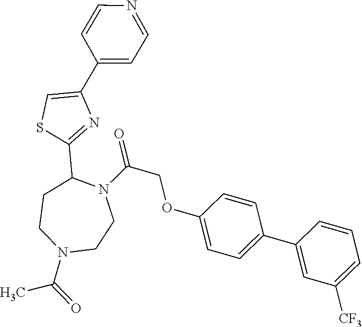 [SMILES: CC(=O)N1CCC(c2nc(-c3ccncc3)cs2)N(C(=O)COc2ccc(-c3cccc(C(F)(F)F)c3)cc2)CC1] | | |
| 66 | 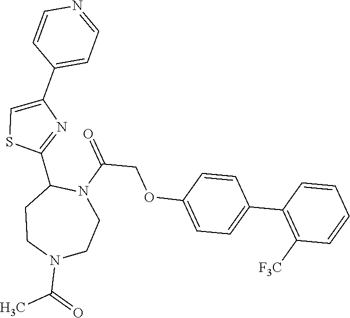 [SMILES: CC(=O)N1CCC(c2nc(-c3ccncc3)cs2)N(C(=O)COc2ccc(-c3ccccc3C(F)(F)F)cc2)CC1] | | |
| 67 | 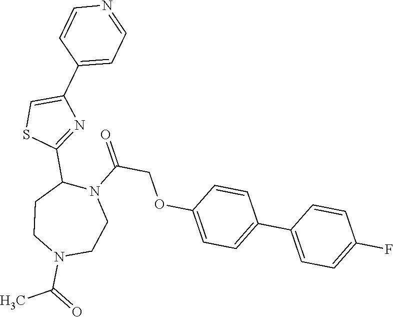 [SMILES: CC(=O)N1CCC(c2nc(-c3ccncc3)cs2)N(C(=O)COc2ccc(-c3ccc(F)cc3)cc2)CC1] | | |
| 68 | 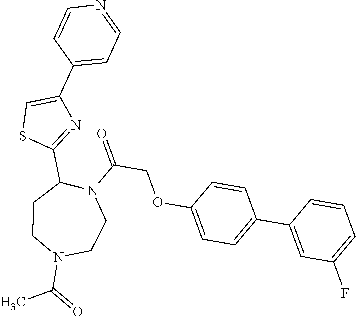 [SMILES: CC(=O)N1CCC(c2nc(-c3ccncc3)cs2)N(C(=O)COc2ccc(-c3cccc(F)c3)cc2)CC1] | | |
| 69 | 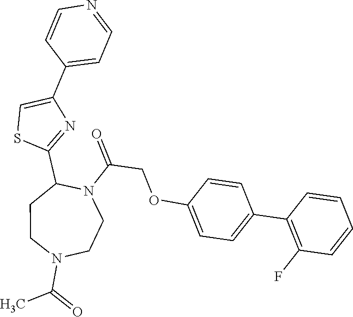 [SMILES: CC(=O)N1CCC(c2nc(-c3ccncc3)cs2)N(C(=O)COc2ccc(-c3ccccc3F)cc2)CC1] | | |
| 70 | 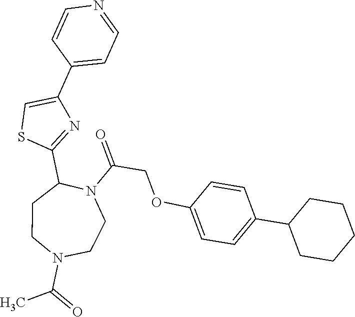 [SMILES: CC(=O)N1CCC(c2nc(-c3ccncc3)cs2)N(C(=O)COc2ccc(C3CCCCC3)cc2)CC1] | | |
| 71 | 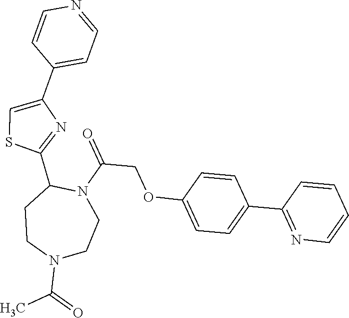 [SMILES: CC(=O)N1CCC(c2nc(-c3ccncc3)cs2)N(C(=O)COc2ccc(-c3ccccn3)cc2)CC1] | | |
| 72 | 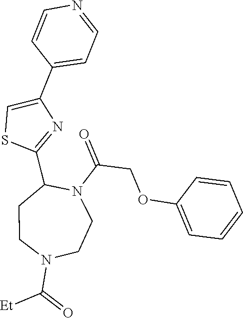 [SMILES: CCC(=O)N1CCC(c2nc(-c3ccncc3)cs2)N(C(=O)COc2ccccc2)CC1] | ¹H NMR (400 MHz, MeOH-d₄) δ 1.05-1.18 (m, 3H), 2.34-2.78 (m, 4H), 3.13-3.27 (m, 0.5H), 3.50-3.77 (m, 3.5H), 3.92-4.44 (m, 3H), 4.93-5.05 (m, 2H), 5.72-5.81 (m, 0.5H), 5.94-6.06 (m, 0.5H), 6.89-7.05 (m, 2H), 7.21-7.33 (m, 2H), 7.94-8.00 (m, 2H), 8.18 (s, 0.21H), 8.19 (s, 0.30H), 8.26 (s, 0.16H), 8.27 (s, 0.24H), 8.56-8.62 (m, 2H). | 451.2 (M + 1) |
| 73 | 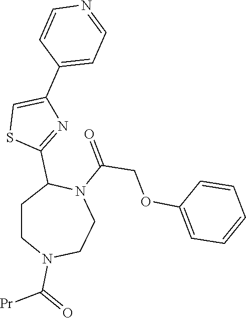 | | |
| 74 | 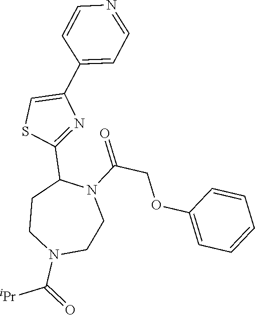 [SMILES: CC(C)C(=O)N1CCC(c2nc(-c3ccncc3)cs2)N(C(=O)COc2ccccc2)CC1] | ¹H NMR (400 MHz, MeOH-d₄) δ 1.05-1.17 (m, 6H), 2.44-2.81 (m, 2H), 2.89-3.08 (m, 1H), 3.13-3.28 (m, 0.5H), 3.51-3.85 (m, 2.5H), 3.89-4.44 (m, 3H), 4.91-5.05 (m, 2H), 5.71-5.81 (m, 0.5H), 5.95-6.05 (m, 0.5H), 6.88-7.05 (m, 3H), 7.20-7.34 (m, 2H), 7.95-8.01 (m, 2H), 8.18 (s, 0.23H), 8.20 (s, 0.33H), 8.27 (s, 0.16H), 8.27 (s, 0.27H), 8.55- 8.63 (m, 2H). | 463.2 (M + 1) |
| 75 | 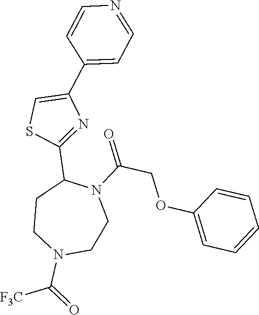 [SMILES: O=C(COc1ccccc1)N1CCN(C(=O)C(F)(F)F)CCC1c1nc(-c2ccncc2)cs1] | | |
| 76 | 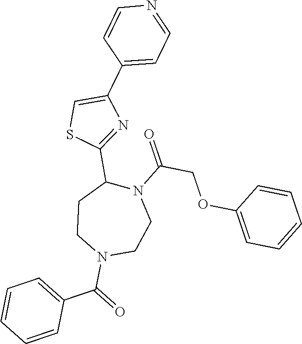 [SMILES: O=C(c1ccccc1)N1CCC(c2nc(-c3ccncc3)cs2)N(C(=O)COc2ccccc2)CC1] | | |
| 77 | 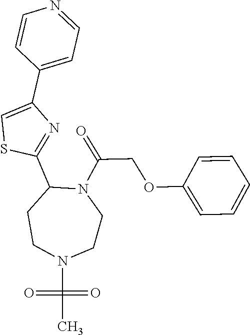 | ¹H NMR (400 MHz, MeOH-d₄) δ 2.45-2.56 (m, 0.5H), 2.61-2.78 (m, 1H), 2.88 (s, 1.35H), 2.93 (s, 1.76H), 3.10-3.24 (m, 1.5H), 3.37-3.53 (m, 1H), 3.74-4.04 (m, 3H), 4.11-4.50 (m, 1H), 4.93-5.07 (m, 2H), 5.66-5.73 (m, 0.5H), 5.90-6.00 (m, 0.5H), 6.87-7.05 (m, 3H), 7.18-7.33 (m, 2H), 7.95-8.02 (m, 2H), 8.18 (s, 0.56H), 8.25 (s, 0.40H), 8.55- 8.63 (m, 2H). | 473.2 (M + 1) |
| 78 | 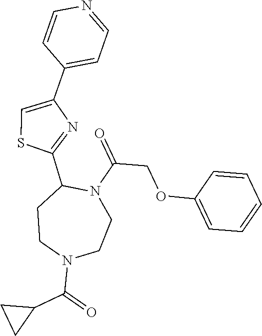 [SMILES: O=C(C1CC1)N1CCC(c2nc(-c3ccncc3)cs2)N(C(=O)COc2ccccc2)CC1] | | |
| 79 | 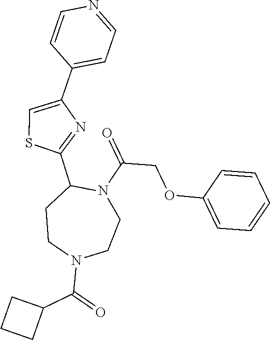 [SMILES: O=C(C1CCC1)N1CCC(c2nc(-c3ccncc3)cs2)N(C(=O)COc2ccccc2)CC1] | | |
| 80 | 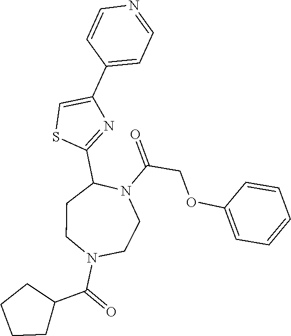 [SMILES: O=C(C1CCCC1)N1CCC(c2nc(-c3ccncc3)cs2)N(C(=O)COc2ccccc2)CC1] | | |
| 81 | 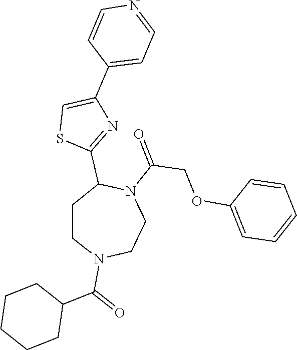 [SMILES: O=C(C1CCCCC1)N1CCC(c2nc(-c3ccncc3)cs2)N(C(=O)COc2ccccc2)CC1] | | |
| 82 | 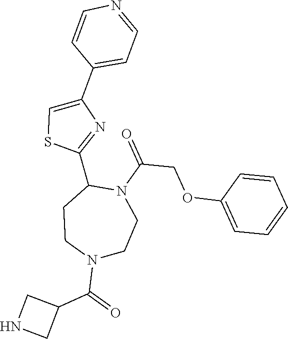 [SMILES: O=C(C1CNC1)N1CCC(c2nc(-c3ccncc3)cs2)N(C(=O)COc2ccccc2)CC1] | | |
| 83 | 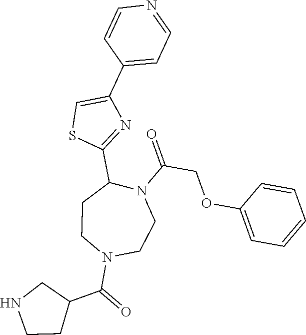 [SMILES: O=C(C1CCNC1)N1CCC(c2nc(-c3ccncc3)cs2)N(C(=O)COc2ccccc2)CC1] | | |
| 84 | 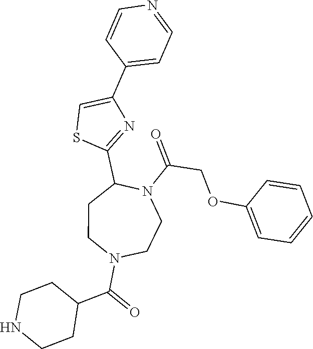 [SMILES: O=C(C1CCNCC1)N1CCC(c2nc(-c3ccncc3)cs2)N(C(=O)COc2ccccc2)CC1] | | |
| 85 | 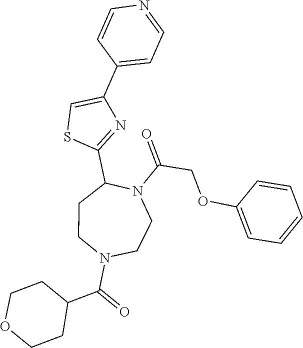 [SMILES: O=C(C1CCOCC1)N1CCC(c2nc(-c3ccncc3)cs2)N(C(=O)COc2ccccc2)CC1] | | |
| 86 | 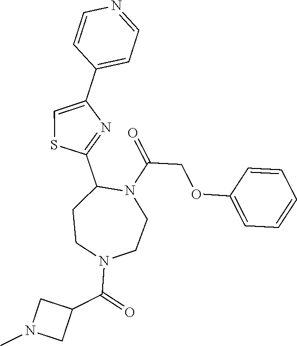 [SMILES: CN1CC(C(=O)N2CCC(c3nc(-c4ccncc4)cs3)N(C(=O)COc3ccccc3)CC2)C1] | | |
| 87 | 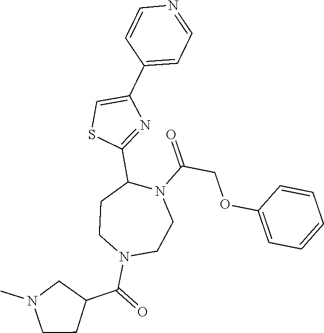 [SMILES: CN1CCC(C(=O)N2CCC(c3nc(-c4ccncc4)cs3)N(C(=O)COc3ccccc3)CC2)C1] | | |
| 88 | 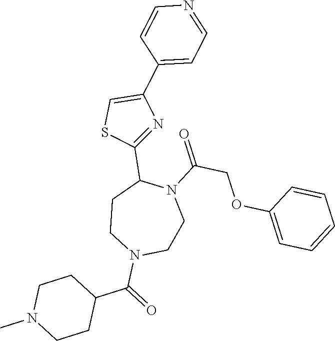 [SMILES: CN1CCC(C(=O)N2CCC(c3nc(-c4ccncc4)cs3)N(C(=O)COc3ccccc3)CC2)CC1] | | |
| 89 | 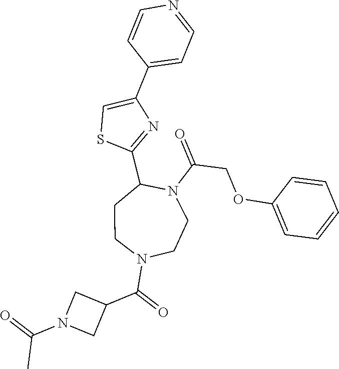 [SMILES: CC(=O)N1CC(C(=O)N2CCC(c3nc(-c4ccncc4)cs3)N(C(=O)COc3ccccc3)CC2)C1] | | |
| 90 | 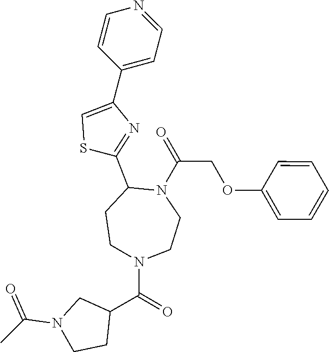 [SMILES: CC(=O)N1CCC(C(=O)N2CCC(c3nc(-c4ccncc4)cs3)N(C(=O)COc3ccccc3)CC2)C1] | | |
| 91 | 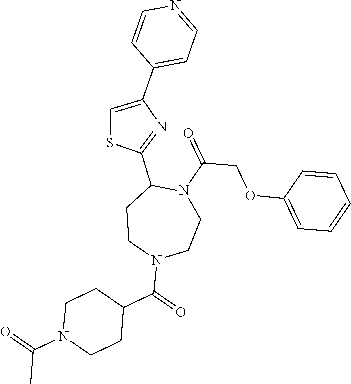 [SMILES: CC(=O)N1CCC(C(=O)N2CCC(c3nc(-c4ccncc4)cs3)N(C(=O)COc3ccccc3)CC2)CC1] | | |
| 92 | 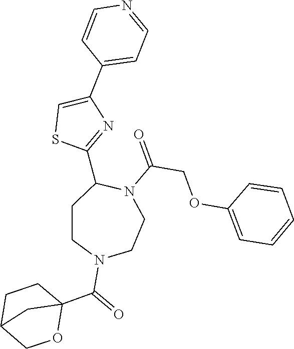 [SMILES: O=C(COc1ccccc1)N1CCN(C(=O)C23CCC(CO2)C3)CCC1c1nc(-c2ccncc2)cs1] | | |
| 93 | 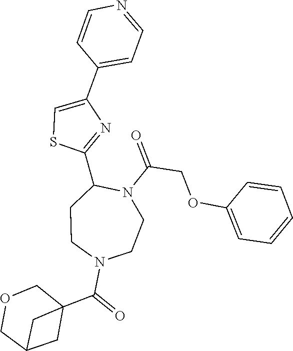 [SMILES: O=C(COc1ccccc1)N1CCN(C(=O)C23COCC(C2)C3)CCC1c1nc(-c2ccncc2)cs1] | | |
| 94 | 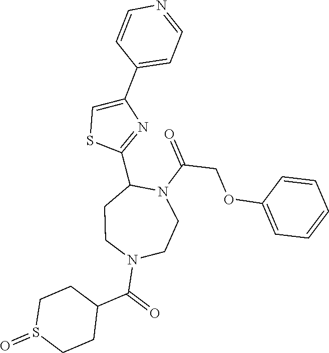 [SMILES: O=C(C1CCS(=O)CC1)N1CCC(c2nc(-c3ccncc3)cs2)N(C(=O)COc2ccccc2)CC1] | | |
| 95 | 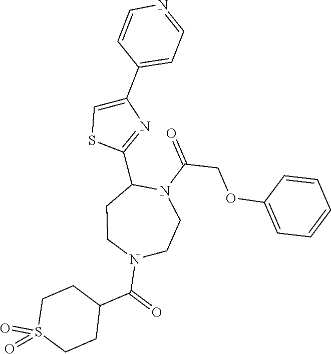 [SMILES: O=C(C1CCS(=O)(=O)CC1)N1CCC(c2nc(-c3ccncc3)cs2)N(C(=O)COc2ccccc2)CC1] | | |
| 96 | 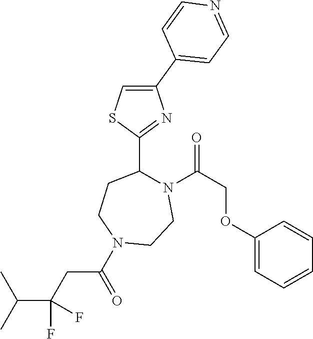 [SMILES: CC(C)C(F)(F)CC(=O)N1CCC(c2nc(-c3ccncc3)cs2)N(C(=O)COc2ccccc2)CC1] | | |
| 97 | 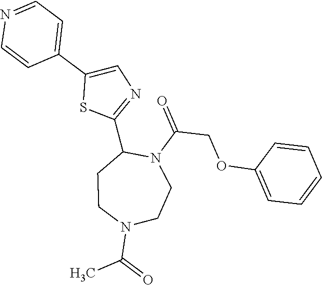 [SMILES: CC(=O)N1CCC(c2ncc(-c3ccncc3)s2)N(C(=O)COc2ccccc2)CC1] | | |
| 98 | 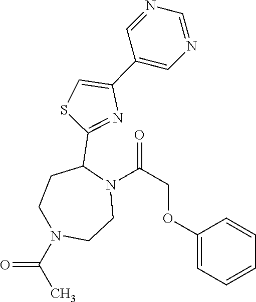 [SMILES: CC(=O)N1CCC(c2nc(-c3cncnc3)cs2)N(C(=O)COc2ccccc2)CC1] | ¹H NMR (400 MHz, MeOH-d₄) δ 2.10 (s, 0.81H), 2.15 (s, 0.52H), 2.16 (s, 0.69H), 2.20 (s, 0.94H), 2.42-2.80 (m, 2H), 3.13-3.29 (m, 0.5H), 3.47-3.82 (m, 2.5H), 3.91-4.43 (m, 3H), 4.93-5.06 (m, 2H), 5.73-5.82 (m, 0.5H), 5.95-6.04 (m, 0.5H), 6.89-7.04 (m, 3H), 7.22-7.34 (m, 2H), 8.16 (s, 0.21H), 8.17 (s, 0.29H), 8.24 (s, 0.17H), 8.24 (s, 0.23H), 9.11- 9.16 (m, 1H), 9.30-9.36 (m, 2H). | 438.0 (M + 1) |
| 99 | 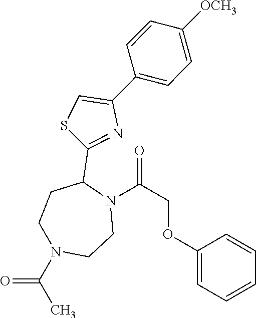 [SMILES: COc1ccc(-c2csc(C3CCN(C(C)=O)CCN3C(=O)COc3ccccc3)n2)cc1] | ¹H NMR (400 MHz, MeOH-d₄) δ 2.09 (s, 0.95H), 2.14 (s, 0.55H), 2.15 (s, 0.57H), 2.19 (s, 0.92H), 2.42-2.78 (m, 2H), 3.13-3.29 (m, 0.5H), 3.47-3.77 (m, 2.5H), 3.85 (s, 3H), 3.97-4.41 (m, 3H), 4.93-5.07 (m, 2H), 5.64-5.74 (m, 0.5H), 5.96-6.07 (m, 0.5H), 6.92-7.04 (m, 5H), 7.22-7.33 (m, 2H), 7.59 (s, 0.20H), 7.61 (s, 0.27H), 7.68 (s, 0.18H), 7.69 (s, 0.27H), 7.83-7.91 (m, 2H). | 466.2 (M + 1) |
| 100 | 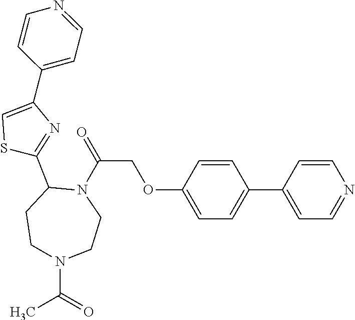 [SMILES: CC(=O)N1CCC(c2nc(-c3ccncc3)cs2)N(C(=O)COc2ccc(-c3ccncc3)cc2)CC1] | ¹H NMR (400 MHz, MeOH-d₄) δ 2.09 (s, 0.78H), 2.15 (s, 0.56H), 2.18 (s, 0.64H), 2.21 (s, 0.92H), 2.42-2.83 (m, 2H), 3.13-3.29 (m, 0.5H), 3.49-3.80 (m, 2.5H), 3.90-4.47 (m, 3H), 5.02-5.15 (m, 2H), 5.73-5.82 (m, 0.5H), 5.95-6.06 (m, 0.5H), 7.03-7.19 (m, 2H), 7.61-7.76 (m, 4H), 7.93-8.00 (m, 2H), 8.18 (s, 0.21H), 8.20 (s, 0.30H), 8.28 (s, 0.16H), 8.28 (s, 0.24H), 8.50- 8.60 (m, 4H). | 514.0 (M + 1) |
| 101 | 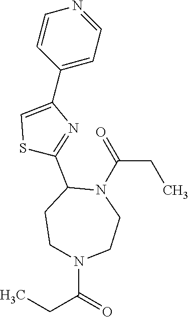 [SMILES: CCC(=O)N1CCC(c2nc(-c3ccncc3)cs2)N(C(=O)CC)CC1] | ¹H NMR (400 MHz, MeOH-d₄) δ 1.06-1.26 (m, 6H), 2.38-2.71 (m, 6H), 3.09-3.30 (m, 0.5H), 3.43-3.72 (m, 2.5H), 3.88-4.44 (m, 3H), 5.61-5.70 (m, 0.5H), 5.95-6.08 (m, 0.5H), 7.93-8.03 (m, 2H), 8.17 (s, 0.25H), 8.19 (s, 0.34H), 8.25 (s, 0.14H), 8.26 (s, 0.21H), 8.53-8.64 (m, 2H). | 373.0 (M + 1) |
| 102 | 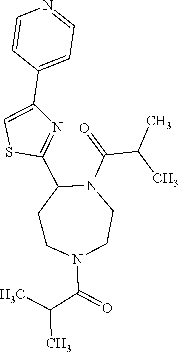 [SMILES: CC(C)C(=O)N1CCC(c2nc(-c3ccncc3)cs2)N(C(=O)C(C)C)CC1] | ¹H NMR (400 MHz, MeOH-d₄) δ 1.06-1.24 (m, 12H), 2.42-2.81 (m, 2H), 2.86-3.21 (m, 2.5H), 3.47-3.79 (m, 2.5H), 3.83-4.41 (m, 3H), 5.72-5.79 (m, 0.5H), 5.96-6.07 (m, 0.5H), 7.93-8.03 (m, 2H), 8.19 (s, 0.27H), 8.20 (s, 0.35H), 8.27 (s, 0.12H), 8.28 (s, 0.24H), 8.53-8.65 (m, 2H). | 401.4 (M + 1) |

### 4. Fatty Acid Uptake Assay for Activity Determination and Efficacy Study in CLP-Induced Mouse Model of Sepsis

4.1 In vitro—Fatty Acid Uptake Assay

### 4.1.1 Description

HEK293 cells were thawed and centrifuged at 400 g in 1 min, whereafter the supernatant was discarded. The cells were resuspended in DMEM+GlutaMax medium (cat #10566016, Invitrogen) containing 10% fetal calf serum (cat #10270-106, Invitrogen) and 1% penicillin/streptomycin (cat #15140-122, Life Technologies). HEK293 cells were seeded into a T25 flask and incubated at 37 degrees Celsius until 80% confluency. When the HEK293 cells reached confluency, cells were counted and seeded in a 96-well plate and with a volume of 10.000 cells/well in medium (as described above) and incubated overnight. Three wells were without cells serving as controls.

The cells were divided into two groups: 1) Example 1, and 2) Untreated. The fatty acid uptake assay (cat #408-100, BioVision) was carried out according to the protocol from BioVision. The only exception to the protocol was that the measurements in the PerkinElmer Multimode Plate Reader Enspire instrument was running overnight.

### 4.1.2 Statistics

Statistics was performed in GraphPad Prism version 8.0. Repeated measure two-way ANOVA were conducted as the groups were tested multiple times followed by a multiple comparison Bonferroni post hoc test.

### 4.1.3 Results of Fatty Acid Uptake Assay

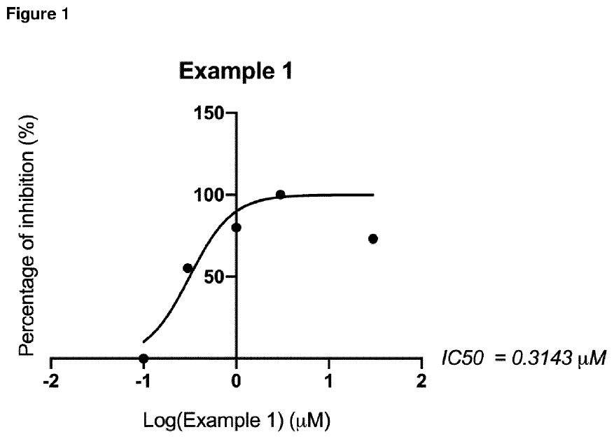

FIG. 1 shows the efficacy of the CPT1 inhibitors, Example 1 (Ex. 1, racemic mixture), tested in the fatty acid uptake assay using HEK293 cells with IC50 of 0.3 μM.

### 4.2 In Vivo—Cecal Ligation and Puncture-Induced Model of Sepsis

### 4.2.1 Description

### Ethical Statement

The Animal Welfare Ethics Review Board of Queen Mary University of London approved all experiments in accordance with the Home Office guidance on the operation of Animals (Scientific Procedures Act 1986) published by Her Majesty's Stationery Office and the Guide for the Care and Use of Laboratory Animals of the National Research Council. Work will be conducted under U.K. home office project license number PC5F29685. All in vivo experiments are reported in accordance to ARRIVE guidelines.

### Animals

This study was be carried out on 10-week old, male C57BL/6 mice (n=5-10 per group) (Charles River Laboratories UK Ltd., Kent, UK) weighing 20-30 g kept under standard laboratory conditions. The animals were allowed to acclimatise to laboratory conditions for at least one week before undergoing experiments. Six mice were housed together in ventilated cages lined with absorbent bedding material. Tubes and chewing blocks were placed in all cages for environmental enrichment. All animals were subjected to 12-h light and dark cycles and the temperature was maintained at 19-23° C. All animals had access to a chow diet and water ad libitum. The cages were cleaned approximately every three days, with water being changed daily. Research staff inspected the animals each day for any signs of illness or abnormal behavior.

### Experimental Design

Ten-week-old, male C57BL/6 mice were injected with buprenorphine (0.05 mg/kg, i.p.). Mice were initially anesthetized by isoflurane (3%) delivered in oxygen at a rate of (1 L/min) in an anesthetic chamber and maintained with isoflurane (2%) and oxygen (1 L/min) via a face mask. Temperature was monitored via a rectal probe and kept at 37° C. by a homeothermic mat. Veet® hair removal cream was used to remove the fur from the abdomen of the mouse and skin was then cleaned with 70% ethanol. The abdomen was opened with a 1.5 cm midline incision to expose the cecum. The cecum was fully ligated below the ileocecal valve, and a G-18 needle was used to puncture two holes in the top and bottom of the cecum. A small amount of faeces was then squeezed out. The cecum was returned to the abdomen in its anatomical position and 5 ml/kg of saline was administered into the abdomen before its closure. Saline (10 ml/kg s.c.) was administered directly after surgery, after CLP and again at 8 h after CLP by oral gavage. At 6 and 18 h after surgery, antibiotics (imipenem/cilastatin; 20 mg/kg dissolved in resuscitation fluid saline s.c.) and an analgesic (buprenorphine; 0.05 mg/kg i.p.) were administered. After 24 h, cardiac function was assessed by echocardiography in vivo. Mice were anaesthetised with isoflurane before performing a cardiac puncture to obtain blood samples. Mice were then killed by removal of the lungs and heart. The organs (heart, lungs, liver, kidney, spleen) and blood was collected for further analysis to quantify organ injury/dysfunction. Mice that underwent sham surgery were not subjected to ligation or perforation of the cecum but were otherwise treated in the same way. The study design is shown in Table 2 below.

TABLE 2
Study design of the in vivo CLP study

| | | Concen- | | | |
|---|---|---|---|---|---|
| Group | Treatment | tration | Vehicle | Time | n |
| A | Sham + | 50:30:20 | PEG400/ | 1 h and 8 h | 5 |
| | vehicle p.o. | | Labrasol/ | | |
| | | | Kolliphor | | |
| B | CLP + | 50:30:20 | PEG400/ | 1 h and 8 h | 10 |
| | vehicle p.o. | | Labrasol/ | | |
| | | | Kolliphor | | |
| C | CLP + | 5 mg/kg, | PEG400/ | 1 h | 10 |
| | Example 1 | racemic | Labrasol/ | | |
| | p.o. | mixture | Kolliphor | | |

### Echocardiography

Echocardiography was conducted at 24 h after CLP under anaesthesia (see below). At the end of the experiment, all mice were sedated by inhalation of 3% isoflurane and 0.4% oxygen and approximately 0.7 ml of blood were obtained via cardiac puncture; the mice were then killed by removal of the heart. Heart, lungs, liver, kidney and spleen were collected and stored at −80° C. for further analyses (see below). The blood samples were centrifuged for 3 min at 9000 RPM and the serum was collected and snap frozen at −80° C.

### Assessment of Cardiac Function In Vivo (Echocardiography)

Cardiac function in mice was assessed by echocardiography in vivo by means of the Vevo 3100 imaging system (VisualSonics, Toronto, Ontario, Canada). Mice were initially anaesthetised with 3% isoflurane (and received oxygen 1 L/min), and then anaesthesia was maintained with 2/o isoflurane and oxygen (1 L/min). Mice were left to stabilise for 10 min before assessment began. During echocardiography, the heart rate was recorded, and a rectal thermometer was recorded to measure core body temperature. Mice were placed on a thermoregulatory platform (set at 42° C.) in the supine position, which enabled us to maintain the body temperature at 37° C. The paws of the animal were taped onto metal EKG leads, which had had electrical conducting gel applied earlier. Hair on the chest of the mouse was removed by hair removal cream. Warmed echo gel was placed onto the shaven chest and the heart was imaged with the probe, whilst the platform was positioned pointing downwards slightly to the left. To obtain the two-dimensional B-mode trace of the left ventricular (LV) the

transducer was placed along the long axis of LV and directed towards the right of the mouse. The probe was then rotated clockwise by 900 to visualise the short axis. Percentage fraction area change (FAC) was calculated from two-dimensional B-mode LV image 100×([LV enddiastolic area—LV end-systolic area)/LV end-diastolic area]. This was done by tracing the endocardial surface of the LV in the parasternal short axis view at the papillary muscles. Onedimensional M-mode images were obtained in the parasternal short axis view of the papillary muscles where the percentage ejection fraction (EF) and fractional shortening (FS) was measured by the following calculations: EF %=100×[(LVIDd3−LVIDs)3)/LVIDd3] and FS %=100×[(LVIDd−LVIDs)/LVIDd]. Images were downloaded and analysed offline using the Vevo software.

### Quantification of Renal Dysfunction and Hepatocellular Injury

Renal dysfunction and hepatocellular injury were analysed in all mice. The mice were anaesthetised with 3% isoflurane and 1% oxygen before being sacrificed. Cardiac puncture was used (G25 needle and non-heparinised syringes) to obtain approximately 0.5 ml of blood.

The blood was immediately decanted into 1.3 ml serum gel tubes (Sarstedt, Nurnbrecht, Germany). The heart and lungs were then removed. The blood samples were centrifuged for 3 min at 9000 RPM to separate the serum, where the 100 μL of serum was pipetted into an aliquot snap frozen in liquid nitrogen and stored at −80° C. for further analysis. The serum was sent to an independent veterinary testing laboratory (MRC Harwell Institute, Oxford, England) to blindly quantify serum urea, creatinine, serum alanine aminotransferase (ALT), serum aspartate aminotransferase (AST) and lactate dehydrogenase (LDH) known markers of renal dysfunction and hepatocellular injury (or in the case of LDH, organ injury).

### 4.2.2 Statistical Analysis

All data in text and figures were expressed as mean±standard error mean (SEM) of n observations. All measurements obtained from the intervention, control and sham were analysed by one-way ANOVA followed by a Bonferroni's post hoc test on GraphPad Prism 8.0 (GraphPad Software, Inc., La Jolla, CA, USA). Differences were considered to be statistically significant when P<0.05.

### 4.2.3 Results of the Efficacy Study in the CLP-Induced Mouse Model of Sepsis

Murine CLP-sepsis was associated with a significant fall in body temperature (hypothermia), systolic and diastolic cardiac dysfunction, renal dysfunction, and hepatocellular injury.

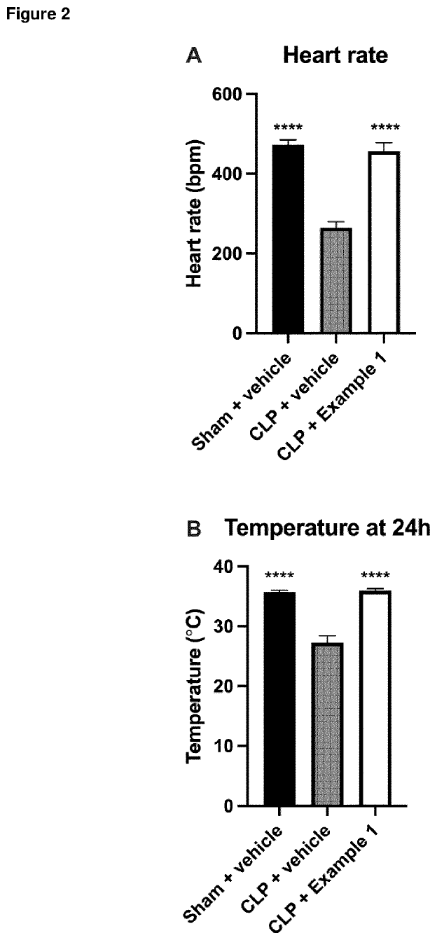

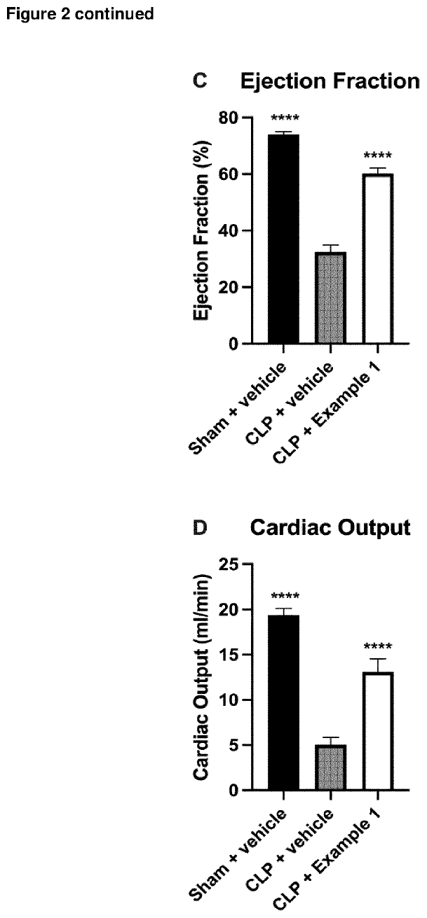

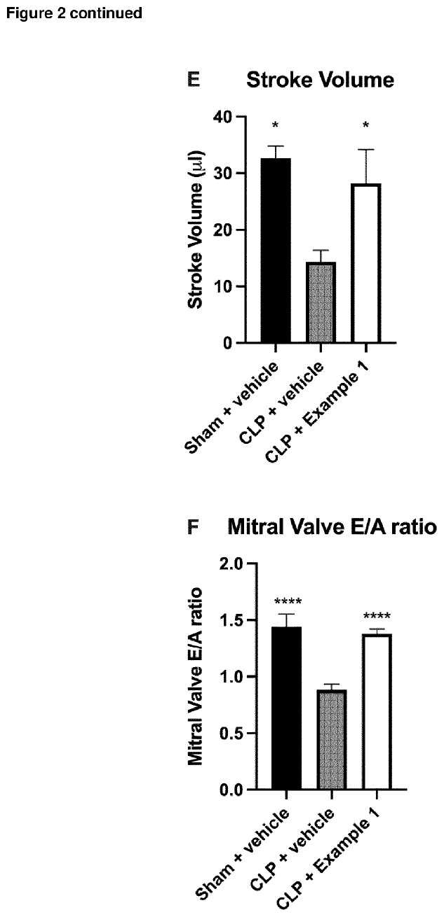

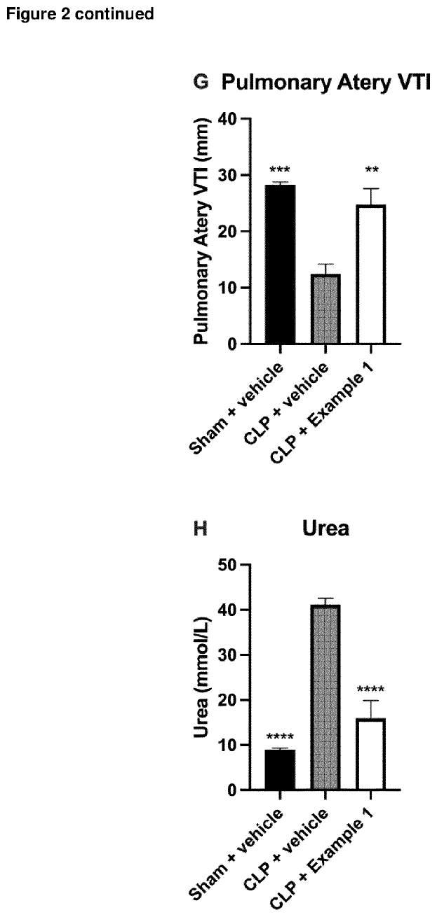

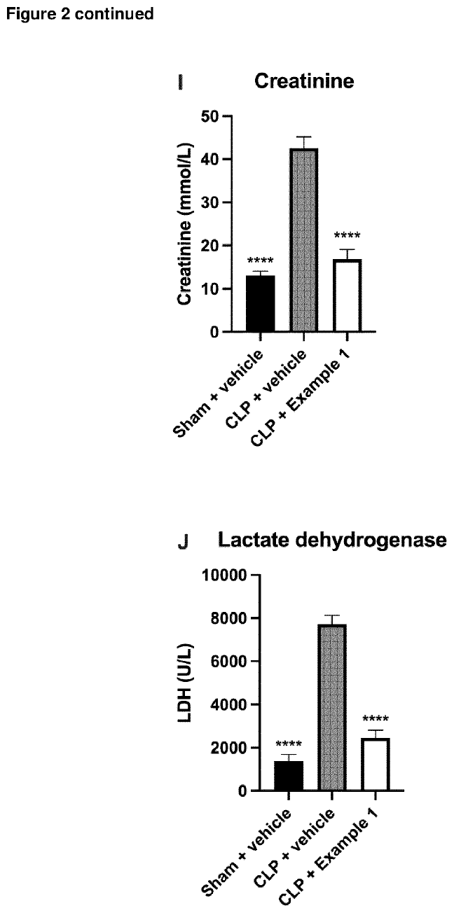

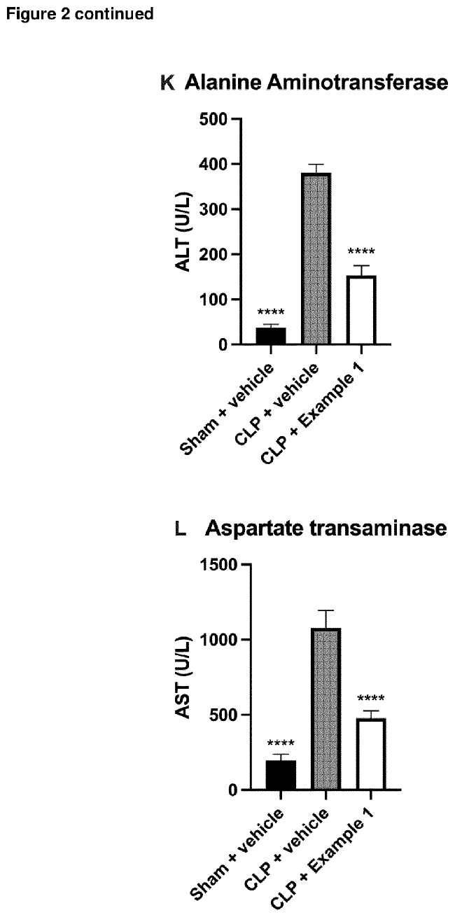

Posttreatment of CLP-mice with Example 1 (5 mg/kg p.o. after CLP) given 1 h after the onset of CLP maintained heart rate (FIG. 2A) and pulmonary artery flow (FIG. 2G), as well as attenuated the hypothermia (FIG. 2B), cardiac dysfunction (systolic and diastolic) (FIG. 2C-F) and renal dysfunction (FIG. 2H-L) caused by CLP-sepsis.

1. A Carnitine-Palmitoyl-Transferase-1 (CPT-1) inhibitor for use in a method of preventing or treating sepsis in a mammalian subject.

2. The Carnitine-Palmitoyl-Transferase-1 (CPT-1) inhibitor for use according to claim 1, wherein the method comprises administering a dosage of Carnitine-Palmitoyl-Transferase-1 (CPT-1) inhibitor to a subject.

3. The Carnitine-Palmitoyl-Transferase-1 (CPT-1) inhibitor for use according to claim 1, wherein the subject is at risk for the sepsis, severe sepsis or septic shock.

4. The Carnitine-Palmitoyl-Transferase-1 (CPT-1) inhibitor for use according to claim 1, wherein the Carnitine-Palmitoyl-Transferase-1 (CPT-1) inhibitor is administered prior to, along with and/or after an event predicted to result in pathogen exposure or introduction of an opportunistic environment.

5. The Carnitine-Palmitoyl-Transferase-1 (CPT-1) inhibitor for use according to claim 4, wherein the Carnitine-Palmitoyl-Transferase-1 (CPT-1) inhibitor is first administered prior to the event, and again on the day of the event, and optionally after the event.

6. The Carnitine-Palmitoyl-Transferase-1 (CPT-1) inhibitor for use of claim 4, wherein the event is selected from admittance to a hospital or health care facility, surgery, placement of an invasive medical device, kidney dialysis, and initiation of chemotherapy or radiation therapy for cancer treatment.

7. The Carnitine-Palmitoyl-Transferase-1 (CPT-1) inhibitor for use according to claim 1, wherein the subject is a human.

8. The Carnitine-Palmitoyl-Transferase-1 (CPT-1) inhibitor for use according to claim 1, wherein the Carnitine-Palmitoyl-Transferase-1 (CPT-1) inhibitor is administered by the oral, sublingual, transdermal or parenteral route, preferably by the intramuscular, intraperitoneal or intravascular route; more preferably by the intravenous route.

9. The Carnitine-Palmitoyl-Transferase-1 (CPT-1) inhibitor for use according to claim 3, wherein in the method the Carnitine-Palmitoyl-Transferase-1 (CPT-1) inhibitor is administered within at least the first 24 hours, 48 hours, 72 hours, or 96 hours of showing one or more signs or symptoms of the sepsis, severe sepsis or septic shock.

10. The Carnitine-Palmitoyl-Transferase-1 (CPT-1) inhibitor for use according to claim 1, wherein the Carnitine-Palmitoyl-Transferase-1 (CPT-1) inhibitor is administered as a single agent.

11. The Carnitine-Palmitoyl-Transferase-1 (CPT-1) inhibitor for use according to claim 1, wherein the Carnitine-Palmitoyl-Transferase-1 (CPT-1) inhibitor is administered in combination with another drug, preferably in combination with an antimicrobial or an antiviral drug.

12. The Carnitine-Palmitoyl-Transferase-1 (CPT-1) inhibitor for use according to claim 1, wherein the Carnitine-Palmitoyl-Transferase-1 (CPT-1) inhibitor is a compound of the formula (I) 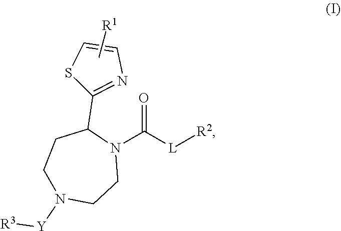
wherein
R¹ is unsubstituted or substituted aryl, preferably unsubstituted or substituted phenyl, naphthyl, tetrahydronaphthyl, indenyl, indanyl, pentalenyl, or fluorenyl,
unsubstituted or substituted heteroaryl, preferably unsubstituted or substituted pyridyl, pyrazinyl, pyrimidinyl, pyridazinyl, 2-oxo-1,2-dihydropyridinyl, oxazolyl, oxydiazolyl, isoxazolyl, thiadiazolyl, tetrazolyl, pyrazolyl, imidazolyl, thiazolyl and thienyl, quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, or indazolyl,
unsubstituted or substituted 5- or 6-membered saturated or partially unsaturated heterocyclyl, or
unsubstituted or substituted C₃-C₈-cycloalkyl, or cyclohexenyl;
L is a single bond, a difunctional linker, preferably *—O—, *—OCH₂—, *—CH₂O—, *—CH₂—, *—CH₂—CH₂—, *—CH₂—CH₂—CH₂—, or *—CH₂—C(CH₃)₂—, or a trifunctional linker, preferably *—CH═, wherein the * indicates the point of attachment to the carbonyl (C═O) group;
R² is unsubstituted or substituted phenyl, naphthyl, or pyridyl, or C₁-C₄-alkyl;
R³ is H, C₁-C₈-alkyl, halogen-C₁-C₄-alkyl, or C₃-C₈-cycloalkyl,
unsubstituted or substituted 4, 5- or 6-membered saturated or partially unsaturated heterocyclyl, or
unsubstituted or substituted phenyl;
Y is —(C═O)—, —(SO₂)— or a single bond;
or a stereoisomer, a pharmaceutically or veterinarily acceptable salt, hydrate or solvate thereof.

13. The Carnitine-Palmitoyl-Transferase-1 (CPT-1) inhibitor for use in a method of preventing or treating sepsis in a mammalian subject, wherein the Carnitine-Palmitoyl-Transferase-1 (CPT-1) inhibitor is a compound of the formula (I) as defined in claim 12, wherein
R¹ is unsubstituted or substituted phenyl, naphthyl, or tetrahydronaphthyl,
unsubstituted or substituted pyridyl, pyrazinyl, pyrimidinyl, oxazolyl, oxydiazolyl, isoxazolyl, thiadiazolyl, tetrazolyl, pyrazolyl, imidazolyl, thiazolyl, thienyl, or quinolinyl,
unsubstituted or substituted pyrrolidinyl, piperidinyl, tetrahydropiperidinyl, or piperazinyl, or
unsubstituted or substituted cyclopentyl; cyclohexyl or cyclohexenyl;
L is a single bond, or a difunctional linker, preferably *—O—, *—OCH₂—, *—CH₂O—, *—CH₂—, or *—CH₂—CH₂—, wherein the * indicates the point of attachment to the carbonyl (C═O) group;
R² is unsubstituted or substituted phenyl, naphthyl, or pyridyl;
R³ is H, C₁-C₄-alkyl, halogen-C₁-C₄-alkyl, cyclopropyl, cyclobutyl, cyclopentyl or cyclohexyl,
unsubstituted or substituted azetidinyl, pyrrolidinyl, piperidinyl, or oxetanyl, 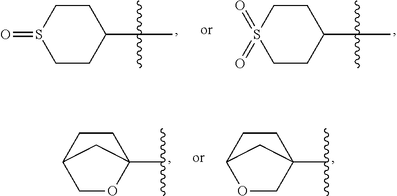 [SMILES: *C1CCS(=O)CC1 ; *C1CCS(=O)(=O)CC1 ; *C12CCC(CO1)C2 ; *C12CCC(C1)OC2]
unsubstituted or substituted phenyl; and
Y is —(C═O)—, —(SO₂)— or a single bond, preferably —(C═O)—.

14. The Carnitine-Palmitoyl-Transferase-1 (CPT-1) inhibitor for use according to claim 1, wherein the Carnitine-Palmitoyl-Transferase-1 (CPT-1) inhibitor is a compound selected from the group consisting of

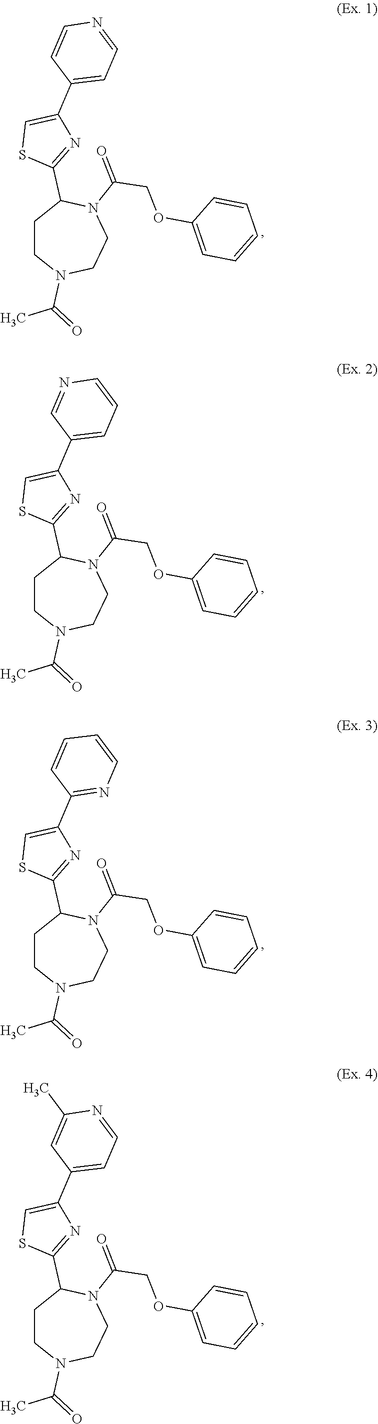 [SMILES: CC(=O)N1CCC(c2nc(-c3ccncc3)cs2)N(C(=O)COc2ccccc2)CC1 ; CC(=O)N1CCC(c2nc(-c3cccnc3)cs2)N(C(=O)COc2ccccc2)CC1 ; CC(=O)N1CCC(c2nc(-c3ccccn3)cs2)N(C(=O)COc2ccccc2)CC1 ; CC(=O)N1CCC(c2nc(-c3ccnc(C)c3)cs2)N(C(=O)COc2ccccc2)CC1]

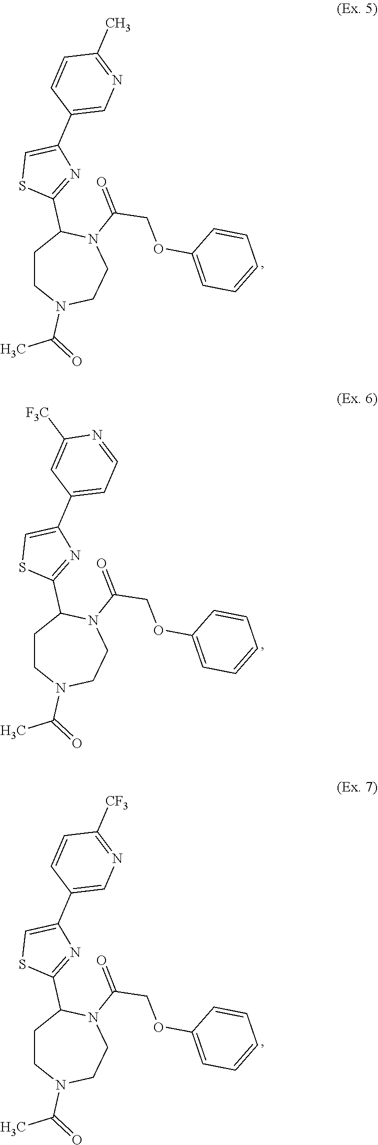 [SMILES: CC(=O)N1CCC(c2nc(-c3ccc(C)nc3)cs2)N(C(=O)COc2ccccc2)CC1 ; CC(=O)N1CCC(c2nc(-c3ccnc(C(F)(F)F)c3)cs2)N(C(=O)COc2ccccc2)CC1 ; CC(=O)N1CCC(c2nc(-c3ccc(C(F)(F)F)nc3)cs2)N(C(=O)COc2ccccc2)CC1]

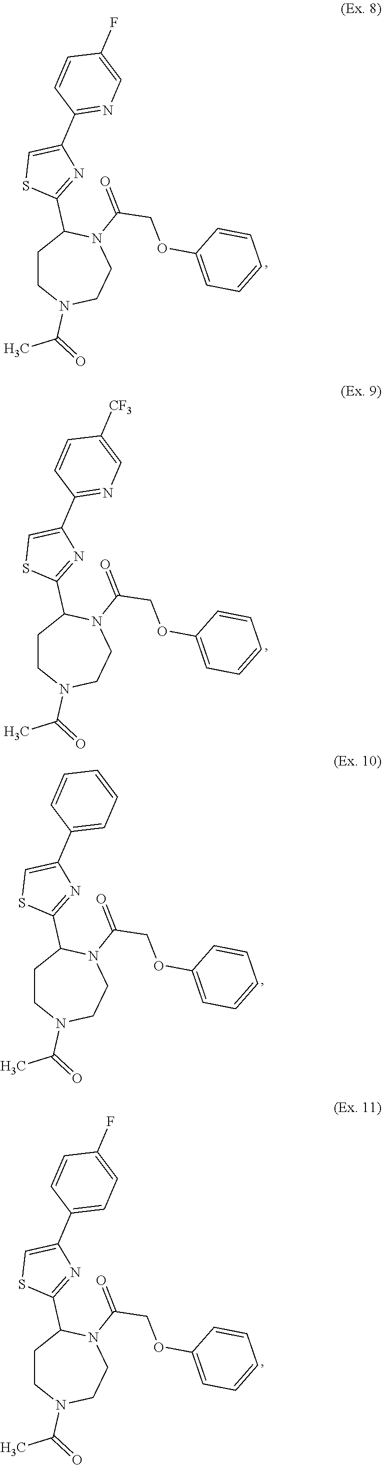

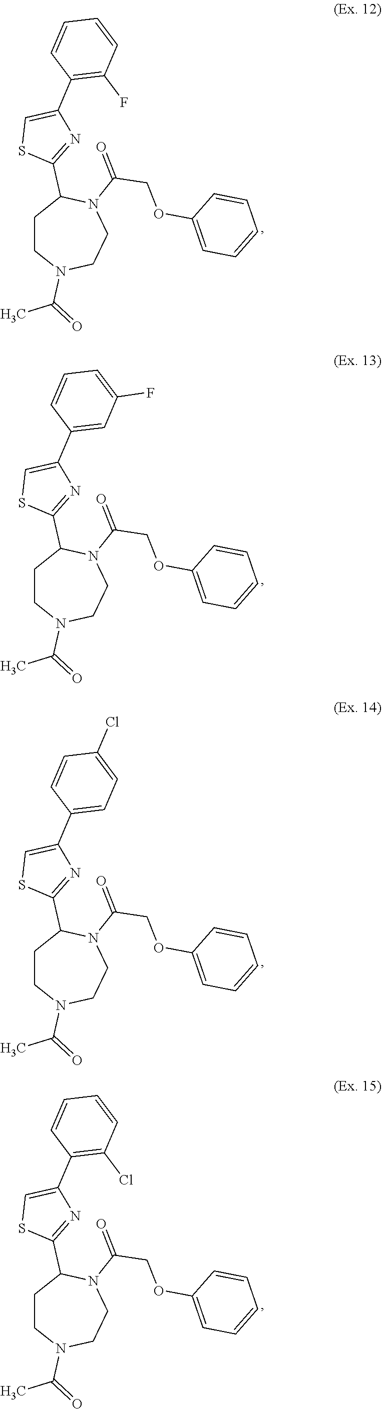

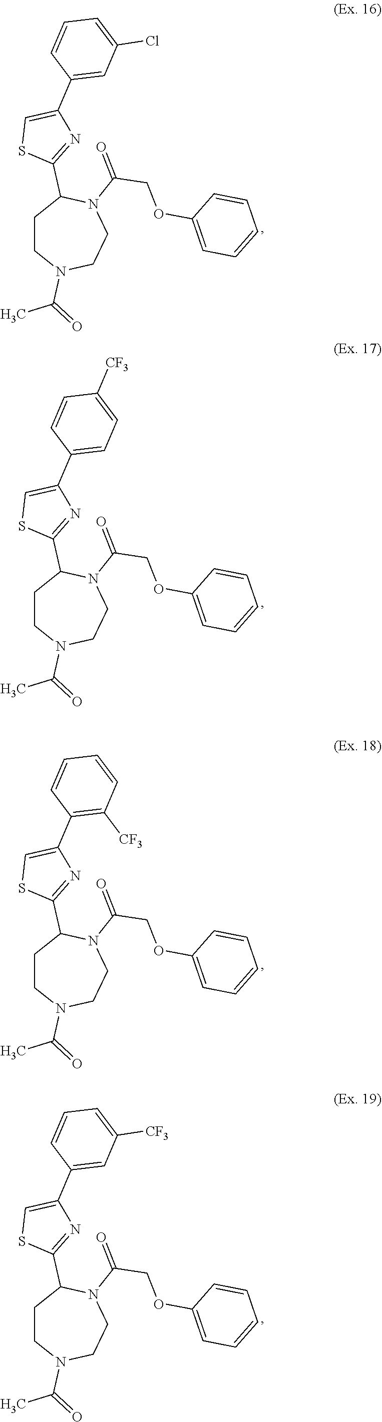 [SMILES: CC(=O)N1CCC(c2nc(-c3cccc(Cl)c3)cs2)N(C(=O)COc2ccccc2)CC1 ; CC(=O)N1CCC(c2nc(-c3ccc(C(F)(F)F)cc3)cs2)N(C(=O)COc2ccccc2)CC1 ; CC(=O)N1CCC(c2nc(-c3ccccc3C(F)(F)F)cs2)N(C(=O)COc2ccccc2)CC1 ; CC(=O)N1CCC(c2nc(-c3cccc(C(F)(F)F)c3)cs2)N(C(=O)COc2ccccc2)CC1]

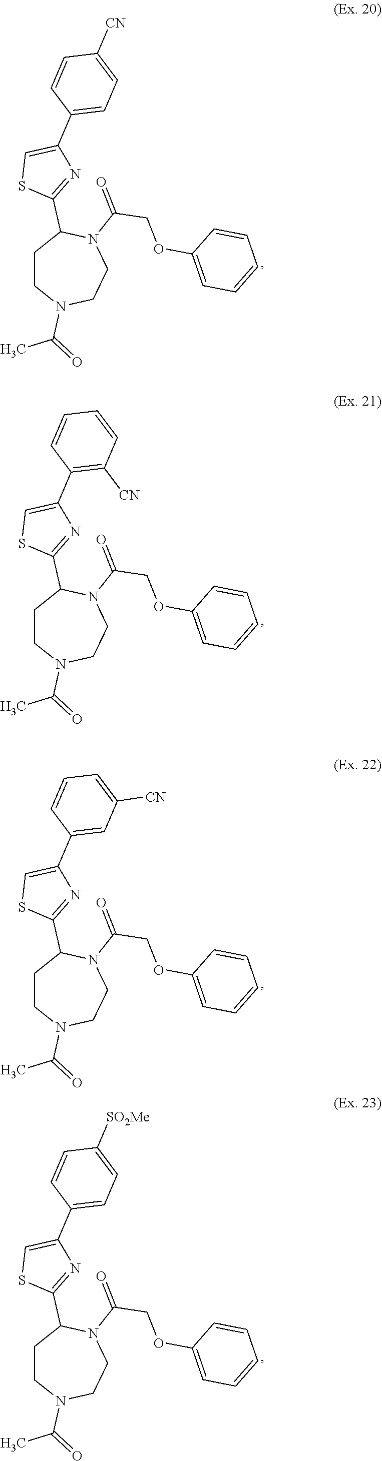 [SMILES: CC(=O)N1CCC(c2nc(-c3ccc(C#N)cc3)cs2)N(C(=O)COc2ccccc2)CC1 ; CC(=O)N1CCC(c2nc(-c3ccccc3C#N)cs2)N(C(=O)COc2ccccc2)CC1 ; CC(=O)N1CCC(c2nc(-c3cccc(C#N)c3)cs2)N(C(=O)COc2ccccc2)CC1 ; CC(=O)N1CCC(c2nc(-c3ccc(S(C)(=O)=O)cc3)cs2)N(C(=O)COc2ccccc2)CC1]

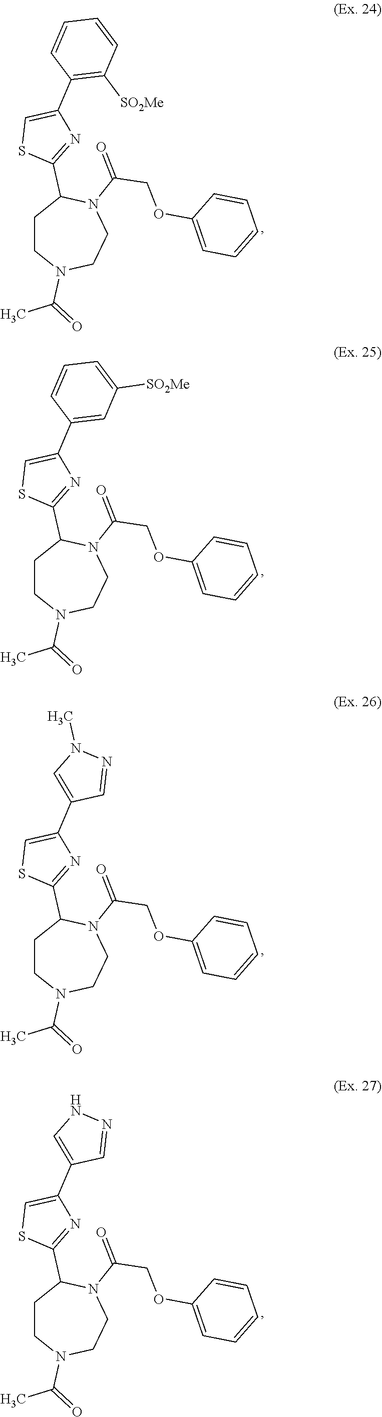

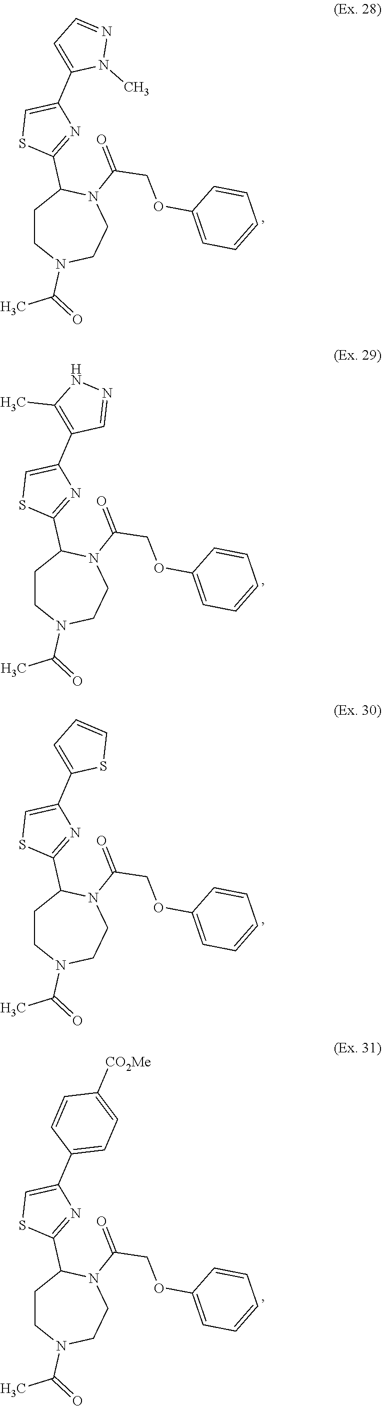

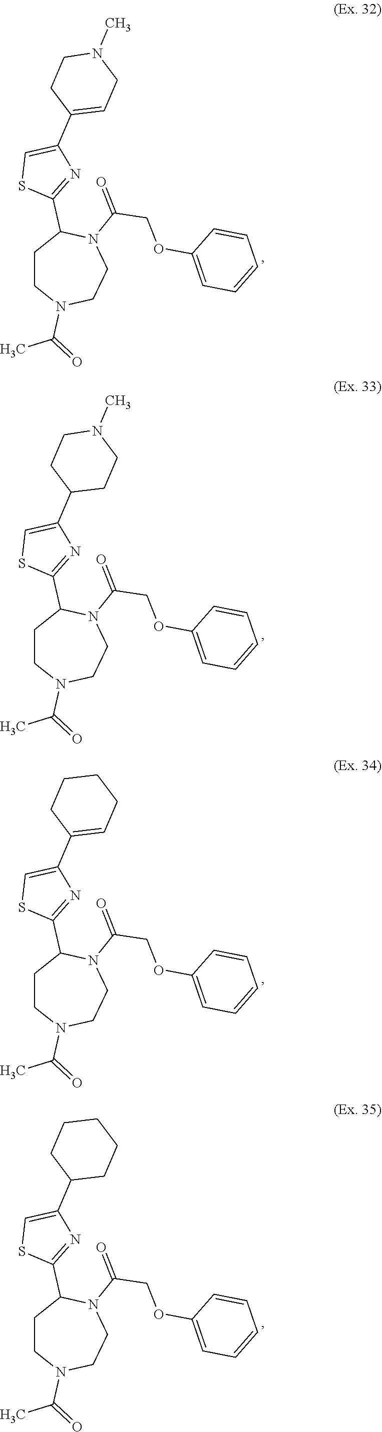

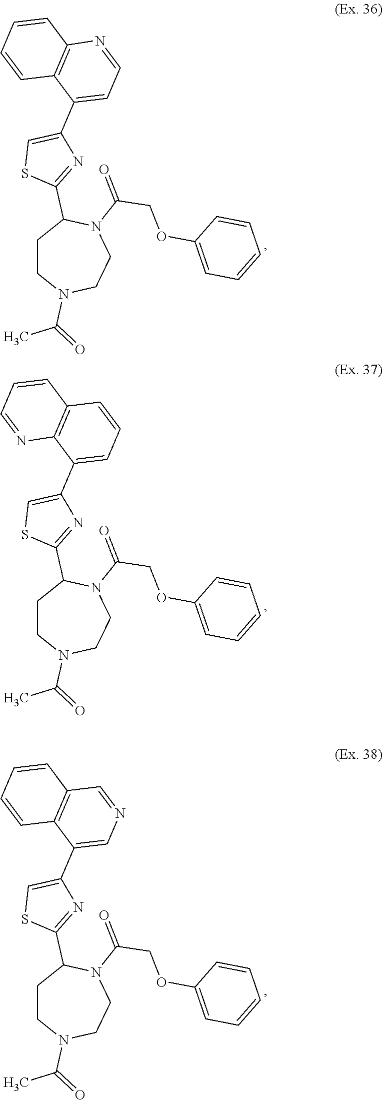

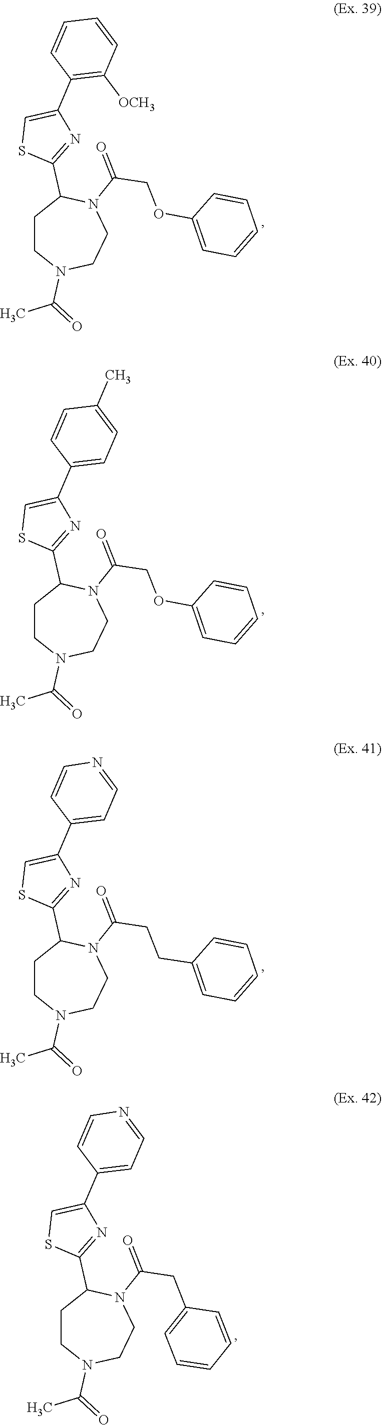 [SMILES: COc1ccccc1-c1csc(C2CCN(C(C)=O)CCN2C(=O)COc2ccccc2)n1 ; CC(=O)N1CCC(c2nc(-c3ccc(C)cc3)cs2)N(C(=O)COc2ccccc2)CC1 ; CC(=O)N1CCC(c2nc(-c3ccncc3)cs2)N(C(=O)CCc2ccccc2)CC1 ; CC(=O)N1CCC(c2nc(-c3ccncc3)cs2)N(C(=O)Cc2ccccc2)CC1]

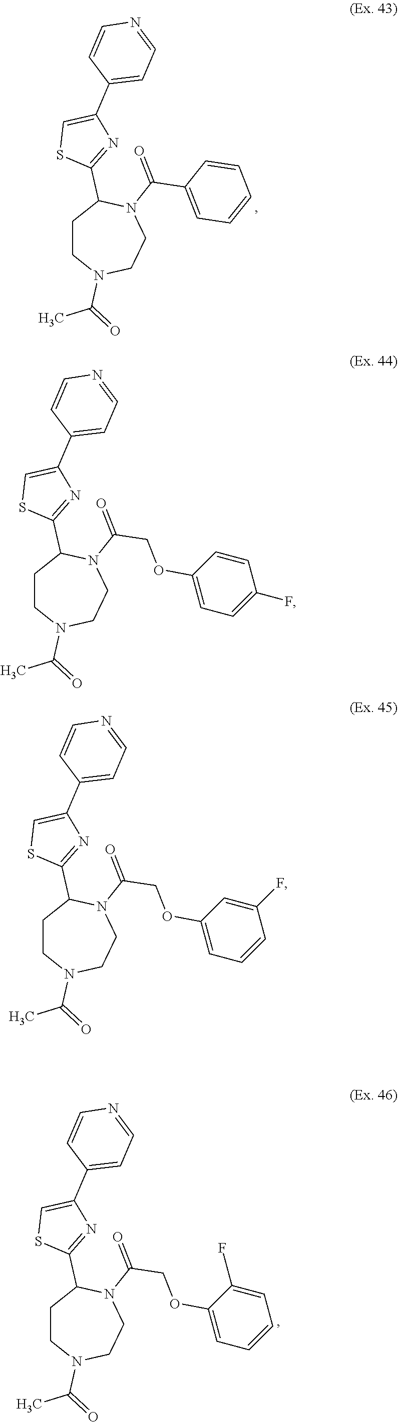

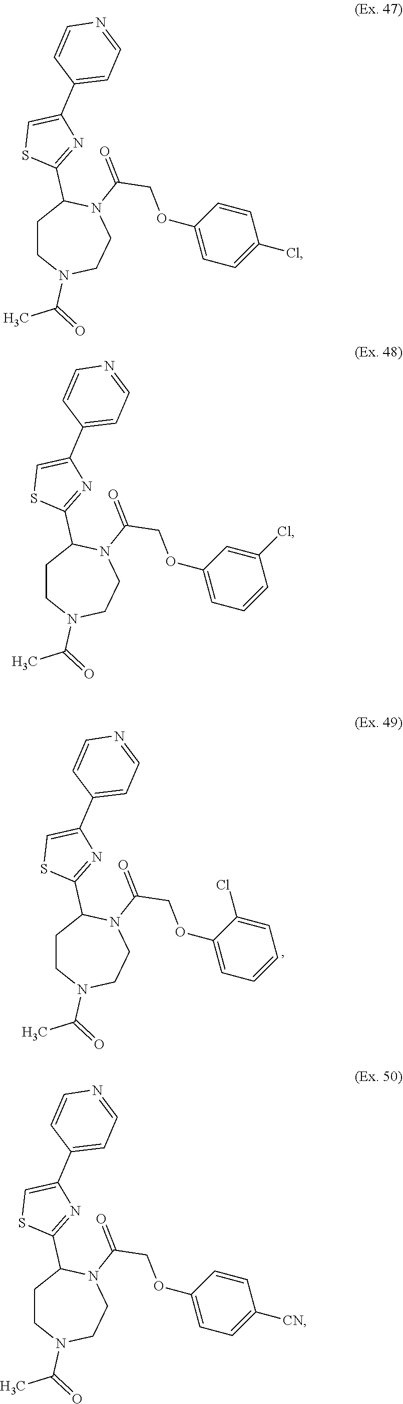

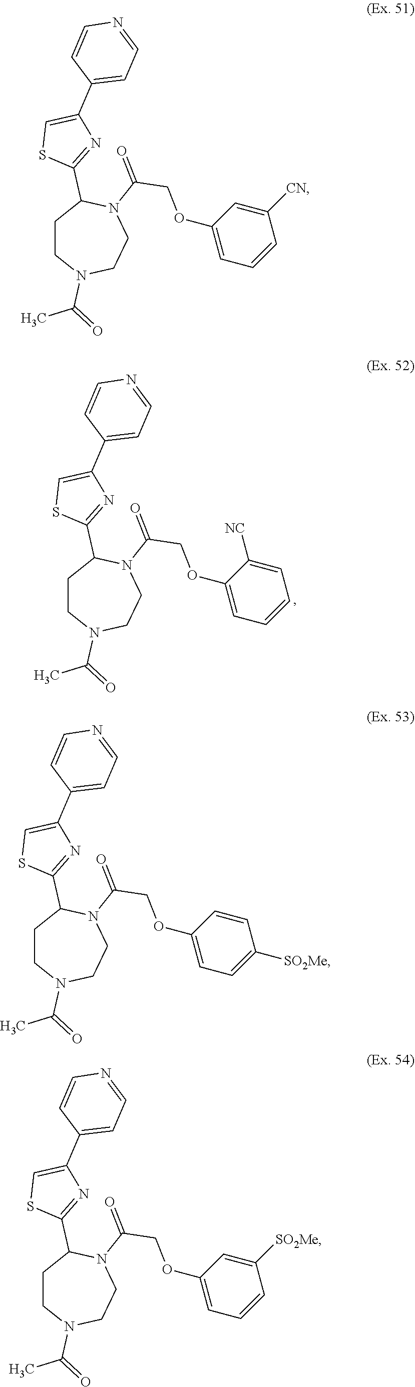

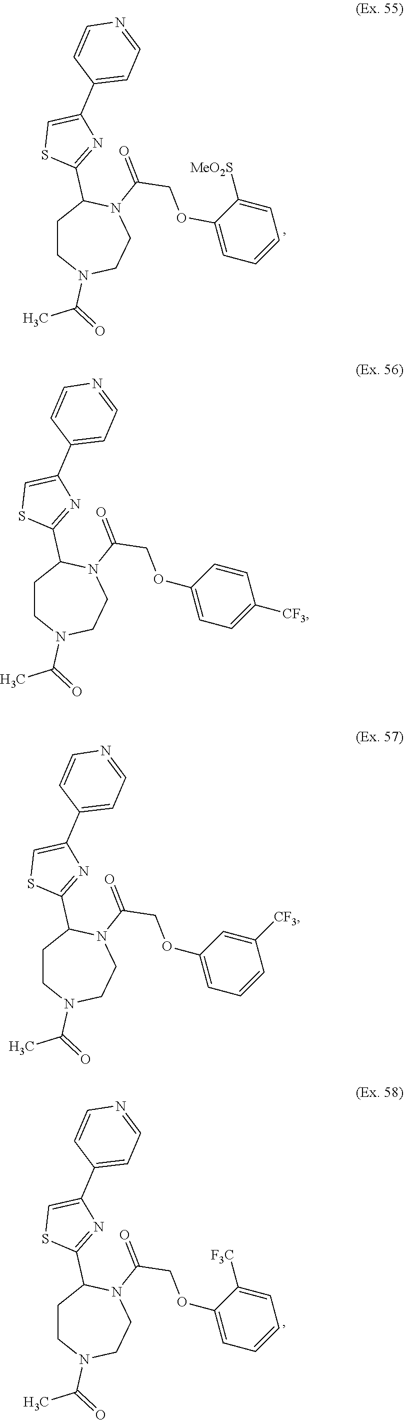 [SMILES: CC(=O)N1CCC(c2nc(-c3ccncc3)cs2)N(C(=O)COc2ccccc2S(C)(=O)=O)CC1 ; CC(=O)N1CCC(c2nc(-c3ccncc3)cs2)N(C(=O)COc2ccc(C(F)(F)F)cc2)CC1 ; CC(=O)N1CCC(c2nc(-c3ccncc3)cs2)N(C(=O)COc2cccc(C(F)(F)F)c2)CC1 ; CC(=O)N1CCC(c2nc(-c3ccncc3)cs2)N(C(=O)COc2ccccc2C(F)(F)F)CC1]

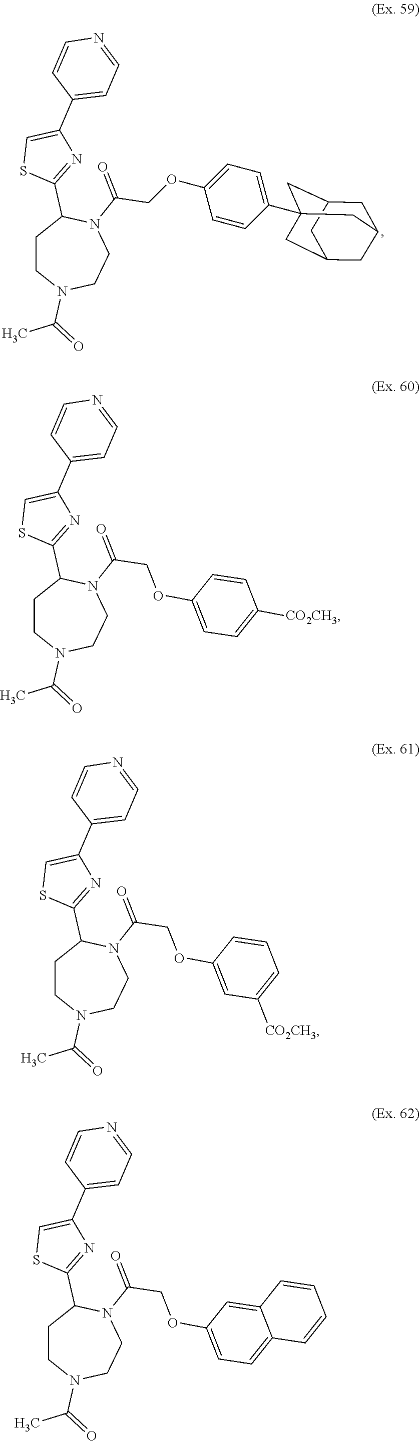 [SMILES: CC(=O)N1CCC(c2nc(-c3ccncc3)cs2)N(C(=O)COc2ccc(C34CC5CC(CC(C5)C3)C4)cc2)CC1 ; COC(=O)c1ccc(OCC(=O)N2CCN(C(C)=O)CCC2c2nc(-c3ccncc3)cs2)cc1 ; COC(=O)c1cccc(OCC(=O)N2CCN(C(C)=O)CCC2c2nc(-c3ccncc3)cs2)c1 ; CC(=O)N1CCC(c2nc(-c3ccncc3)cs2)N(C(=O)COc2ccc3ccccc3c2)CC1]

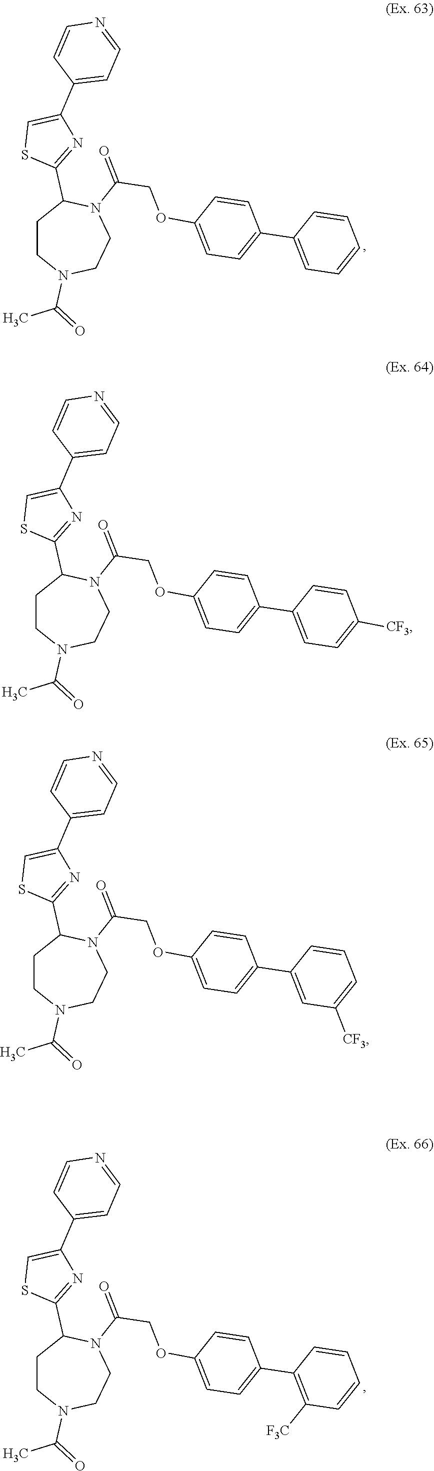 [SMILES: CC(=O)N1CCC(c2nc(-c3ccncc3)cs2)N(C(=O)COc2ccc(-c3ccccc3)cc2)CC1 ; CC(=O)N1CCC(c2nc(-c3ccncc3)cs2)N(C(=O)COc2ccc(-c3ccc(C(F)(F)F)cc3)cc2)CC1 ; CC(=O)N1CCC(c2nc(-c3ccncc3)cs2)N(C(=O)COc2ccc(-c3cccc(C(F)(F)F)c3)cc2)CC1 ; CC(=O)N1CCC(c2nc(-c3ccncc3)cs2)N(C(=O)COc2ccc(-c3ccccc3C(F)(F)F)cc2)CC1]

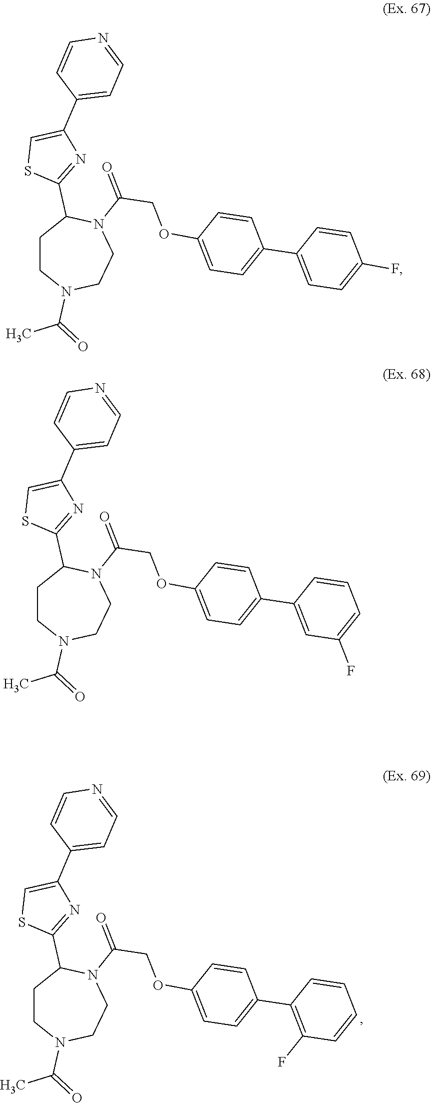

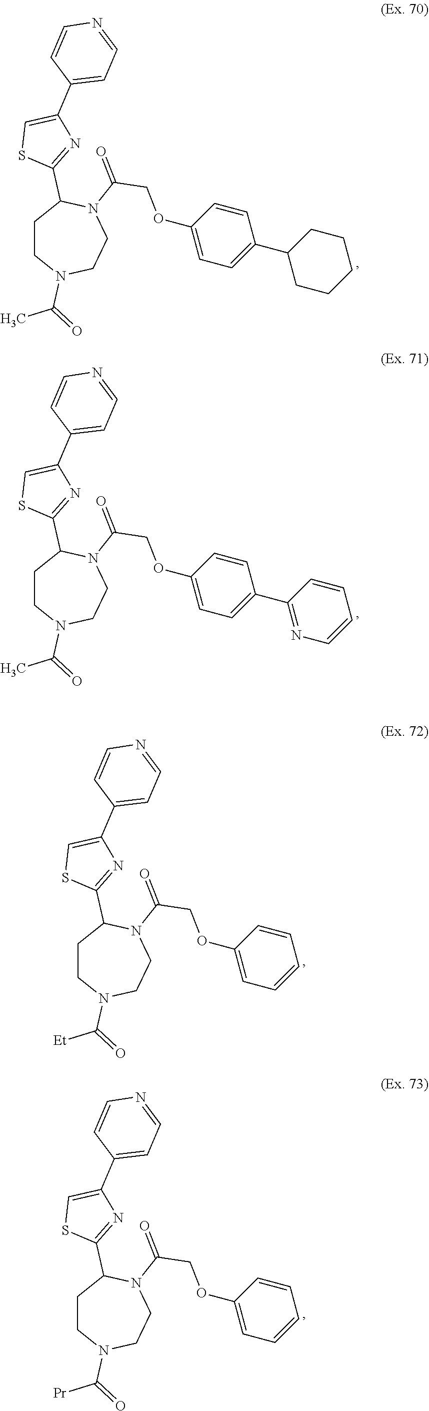

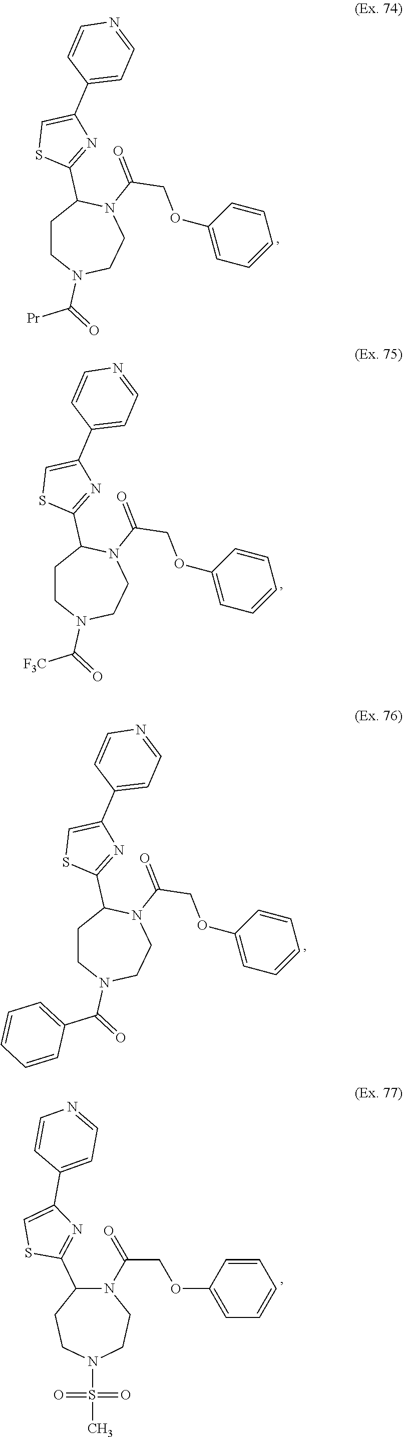

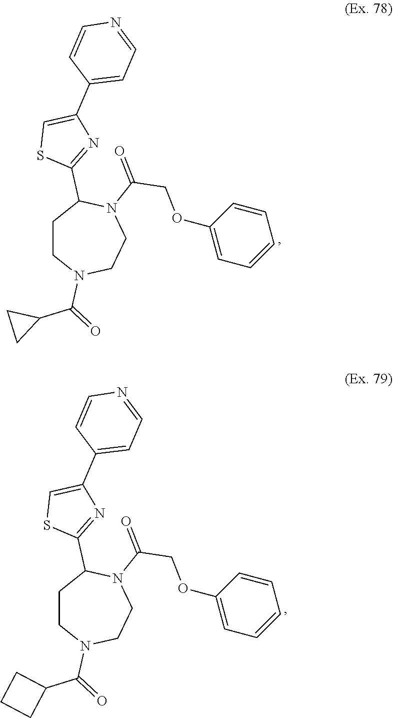 [SMILES: O=C(C1CC1)N1CCC(c2nc(-c3ccncc3)cs2)N(C(=O)COc2ccccc2)CC1 ; O=C(C1CCC1)N1CCC(c2nc(-c3ccncc3)cs2)N(C(=O)COc2ccccc2)CC1]

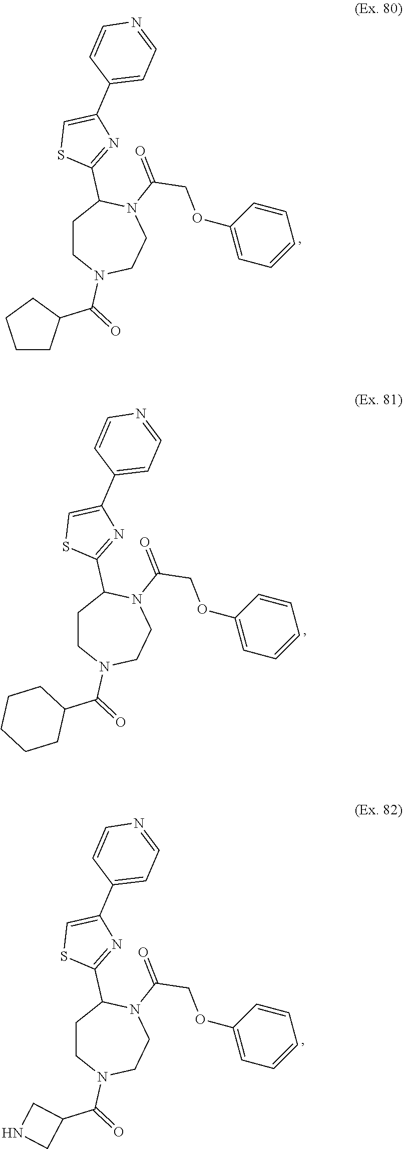 [SMILES: O=C(C1CCCC1)N1CCC(c2nc(-c3ccncc3)cs2)N(C(=O)COc2ccccc2)CC1 ; O=C(C1CCCCC1)N1CCC(c2nc(-c3ccncc3)cs2)N(C(=O)COc2ccccc2)CC1 ; O=C(C1CNC1)N1CCC(c2nc(-c3ccncc3)cs2)N(C(=O)COc2ccccc2)CC1]

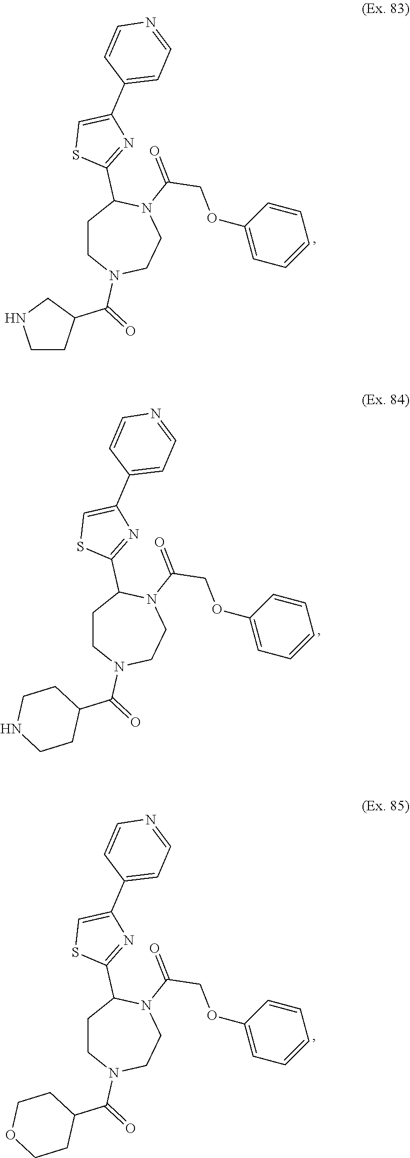 [SMILES: O=C(C1CCNC1)N1CCC(c2nc(-c3ccncc3)cs2)N(C(=O)COc2ccccc2)CC1 ; O=C(C1CCNCC1)N1CCC(c2nc(-c3ccncc3)cs2)N(C(=O)COc2ccccc2)CC1 ; O=C(C1CCOCC1)N1CCC(c2nc(-c3ccncc3)cs2)N(C(=O)COc2ccccc2)CC1]

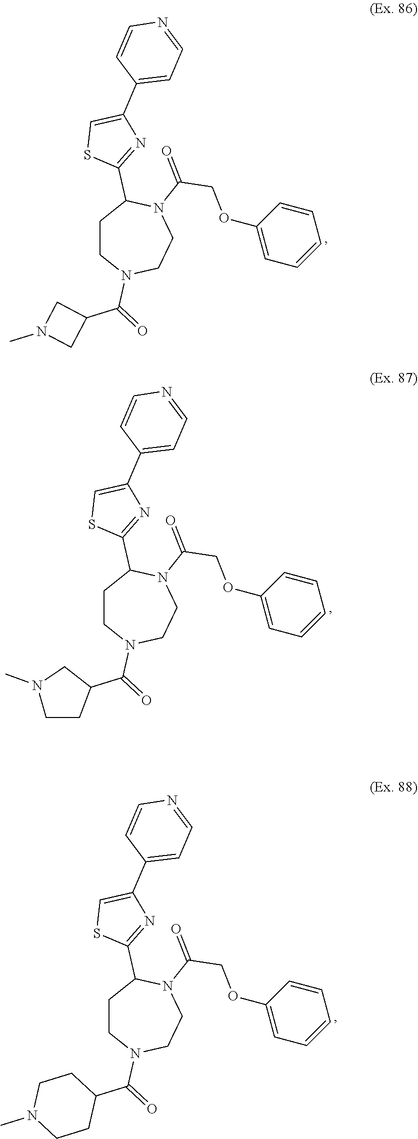 [SMILES: CN1CC(C(=O)N2CCC(c3nc(-c4ccncc4)cs3)N(C(=O)COc3ccccc3)CC2)C1 ; CN1CCC(C(=O)N2CCC(c3nc(-c4ccncc4)cs3)N(C(=O)COc3ccccc3)CC2)C1 ; CN1CCC(C(=O)N2CCC(c3nc(-c4ccncc4)cs3)N(C(=O)COc3ccccc3)CC2)CC1]

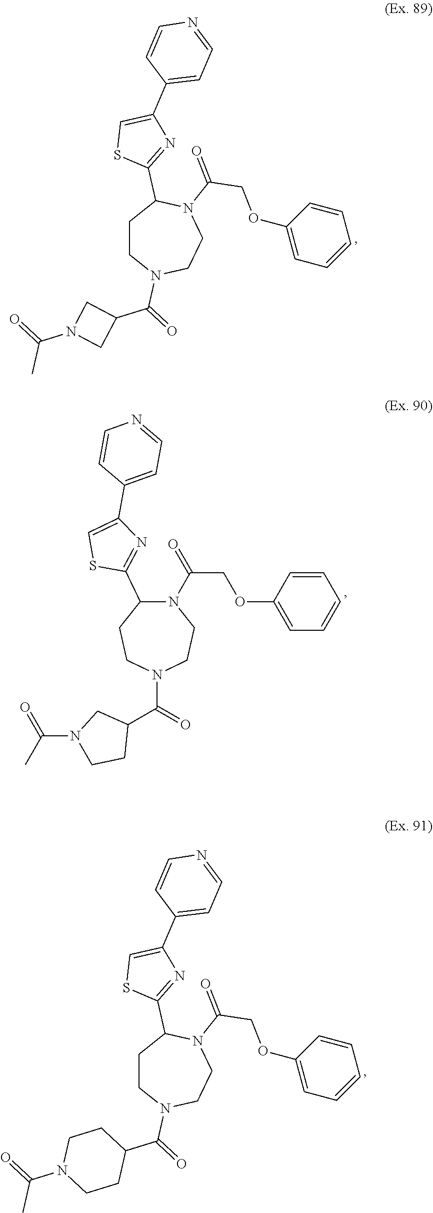 [SMILES: CC(=O)N1CC(C(=O)N2CCC(c3nc(-c4ccncc4)cs3)N(C(=O)COc3ccccc3)CC2)C1 ; CC(=O)N1CCC(C(=O)N2CCC(c3nc(-c4ccncc4)cs3)N(C(=O)COc3ccccc3)CC2)C1 ; CC(=O)N1CCC(C(=O)N2CCC(c3nc(-c4ccncc4)cs3)N(C(=O)COc3ccccc3)CC2)CC1]

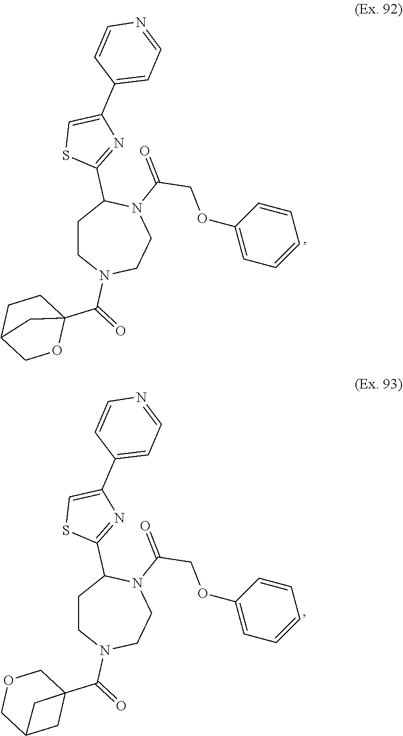 [SMILES: O=C(COc1ccccc1)N1CCN(C(=O)C23CCC(CO2)C3)CCC1c1nc(-c2ccncc2)cs1 ; O=C(COc1ccccc1)N1CCN(C(=O)C23COCC(C2)C3)CCC1c1nc(-c2ccncc2)cs1]

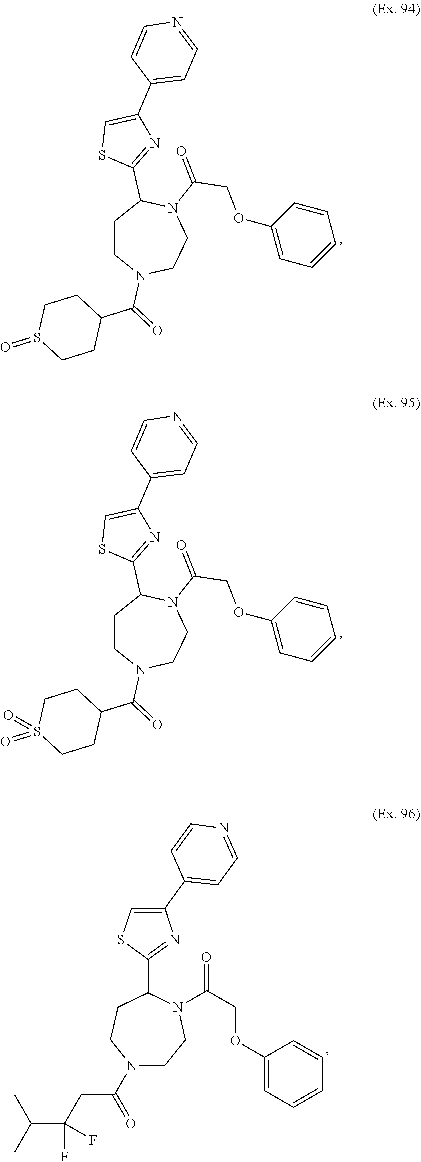 [SMILES: O=C(C1CCS(=O)CC1)N1CCC(c2nc(-c3ccncc3)cs2)N(C(=O)COc2ccccc2)CC1 ; O=C(C1CCS(=O)(=O)CC1)N1CCC(c2nc(-c3ccncc3)cs2)N(C(=O)COc2ccccc2)CC1 ; CC(C)C(F)(F)CC(=O)N1CCC(c2nc(-c3ccncc3)cs2)N(C(=O)COc2ccccc2)CC1]

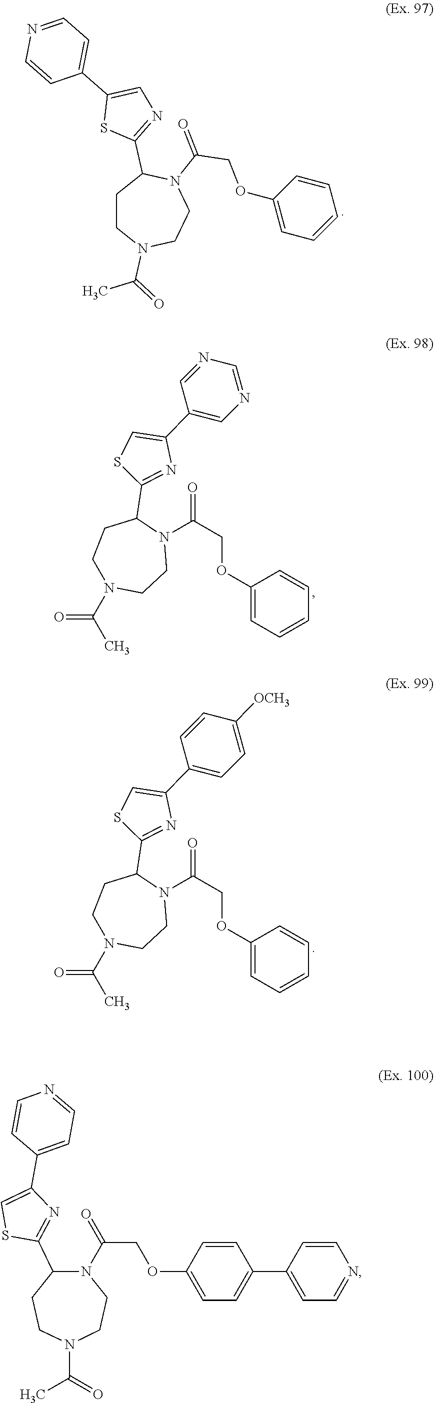 [SMILES: CC(=O)N1CCC(c2ncc(-c3ccncc3)s2)N(C(=O)COc2ccccc2)CC1 ; CC(=O)N1CCC(c2nc(-c3cncnc3)cs2)N(C(=O)COc2ccccc2)CC1 ; COc1ccc(-c2csc(C3CCN(C(C)=O)CCN3C(=O)COc3ccccc3)n2)cc1 ; CC(=O)N1CCC(c2nc(-c3ccncc3)cs2)N(C(=O)COc2ccc(-c3ccncc3)cc2)CC1]

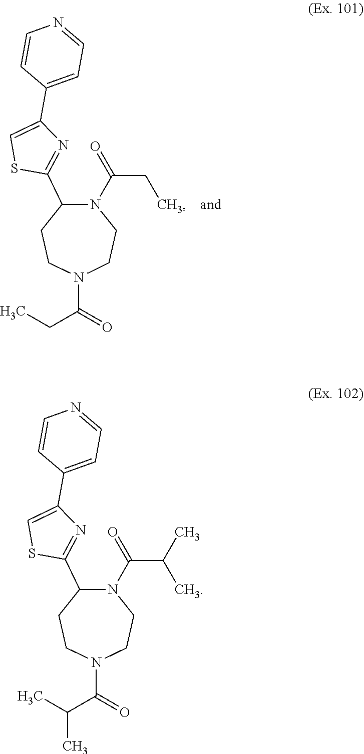 [SMILES: CCC(=O)N1CCC(c2nc(-c3ccncc3)cs2)N(C(=O)CC)CC1 ; "and" ; CC(C)C(=O)N1CCC(c2nc(-c3ccncc3)cs2)N(C(=O)C(C)C)CC1]

15. A pharmaceutical composition comprising the Carnitine-Palmitoyl-Transferase-1 (CPT-1) inhibitor as defined in claim 12 for use in a method of preventing or treating sepsis in a mammalian subject, further comprising a pharmaceutically acceptable excipient.
